(19) United States
(12) Patent Application Publication
(10) Pub. No.: US 2020/0002283 A1
(43) Pub. Date: Jan. 2, 2020

(54) PYRAZOLE DERIVATIVES HAVING ACTIVITY AGAINST PAIN

(71) Applicant: ESTEVE PHARMACEUTICALS, S.A., Barcelona (ES)

(72) Inventors: Carmen ALMANSA-ROSALES, Barcelona (ES); Susana YENES-MÍNGUEZ, Molins de Rei (ES); José-Luis DÍAZ-FERNÁNDEZ, Manresa (ES); Antonio David RODRIGUEZ-GARRIDO, Santiago de Compostela (ES)

(21) Appl. No.: 16/307,549

(22) PCT Filed: Jun. 26, 2017
(86) PCT No.: PCT/EP2017/065730

§ 371 Date: Dec. 6, 2018

(30) Foreign Application Priority Data
Jun. 29, 2016 (EP) 16382306.5

(51) Int. Cl. C07D 231/12; C07D 401/04; C07D 403/04; C07D 401/06; C07D 413/06; C07D 417/12

(52) U.S. Cl. CPC C07D 231/12; C07D 401/04; C07D 417/12; C07D 401/06; C07D 413/06; C07D 403/04

(57) ABSTRACT

The present invention relates to pyrazole derivatives having pharmacological activity towards the α2δ subunit, in particular the α2δ-1 subunit, of the voltage-gated calcium channel, in particular having dual pharmacological activity towards both the α2δ subunit, in particular the α2δ-1 subunit, of the voltage-gated calcium channel and the μ-opioid receptor. The present invention also relates to processes of preparation of such compounds, to pharmaceutical compositions comprising them, and to their use in therapy, in particular for the treatment of pain.

### FIELD OF THE INVENTION

The present invention relates to compounds having pharmacological activity towards the α₂δ subunit of the voltage-gated calcium channel. In particular, the present invention relates to compounds having dual pharmacological activity towards both the α₂δ subunit of the voltage-gated calcium channel, and the μ-opioid receptor (MOR or mu-opioid receptor). More particularly, the present invention relates to pyrazole derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved (Turk, D. C., Wilson, H. D., Cahana, A.; 2011; Lancet; 377; 2226-2235). Pain affects a big portion of the population with an estimated prevalence of 20% and its incidence, particularly in the case of chronic pain, is increasing due to the population ageing. Additionally, pain is clearly related to comorbidities, such as depression, anxiety and insomnia, which lead to important productivity losses and socio-economical burden (Goldberg, D. S., McGee, S. J.; 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al., Pharmacol Rev. 2015 67:821-70). The VGCC are assembled through interactions of different subunits, namely α₁ (Caᵥα₁), β(Caᵥβ) α₂δ (Caᵥα₂δ) and γ (Caᵥγ). The α₁ subunits are the key porous forming units of the channel complex, being responsible for the Ca²⁺ conduction and generation of Ca²⁺ influx. The or β₂δ, β, and γ subunits are auxiliary, although very important for the regulation of the channel since they increase the expression of the α₁ subunits in the plasma membrane as well as modulate their function, resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N—(Caᵥ2.2), P/Q-(Caᵥ2.1), and R—(Caᵥ2.3) types, depending on the channel forming Caᵥa subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo, Neurotherapeutics. 2009 6:679-92; Zamponi et al., 2015 supra; Neumaier et al., Prog Neurobiol. 2015 129:1-36.). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain. (Perret and Luo, 2009, supra; Vink and Alewood, Br J Pharmacol. 2012 167:970-89.).

The Caᵥ1 and Caᵥ2 subfamilies contain an auxiliary α₂δ subunit, which is the therapeutic target of the gabapentinoid drugs of value in certain epilepsies and chronic neuropathic pain. To date, there are four known α₂δ subunits, each encoded by a unique gene and all possessing splice variants. Each α₂δ protein is encoded by a single messenger RNA and is posttranslationally cleaved and then linked by disulfide bonds. Four genes encoding α₂δ subunits have now been cloned. α₂δ-1 was initially cloned from skeletal muscle and shows a fairly ubiquitous distribution. The α₂δ-2 and α₂δ-3 subunits were subsequently cloned from brain. The most recently identified subunit, α₂δ-4, is largely nonneuronal. The human α₂δ-4 protein sequence shares 30, 32 and 61% identity with the human α₂δ-1, α₂δ-2 and α₂δ-3 subunits, respectively. The gene structure of all α₂δ subunits is similar. All α₂δ subunits show several splice variants (Davies et al., Trends Pharmacol Sci. 2007 28:220-8.; Dolphin A C, Nat Rev Neurosci. 2012 13:542-55., Biochim Biophys Acta. 2013 1828:1541-9.).

The Caᵥα₂δ-1 subunit may play an important role in neuropathic pain development (Perret and Luo, 2009, supra; Vink and Alewood, 2012, supra). Biochemical data have indicated a significant Caᵥα₂δ-1, but not Caᵥα₂δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia in nerve injured animals, suggesting that elevated DRG Caᵥα₂δ-1 subunit contributes to neuropathic allodynia.

The Caᵥα₂δ-1 subunit (and the Caᵥα₂δ-2, but not Caᵥα₂δ-3 and Caᵥα₂δ-4, subunits) is the binding site for gabapentin which has anti-allodynic/hyperalgesic properties in patients and animal models. Because injury-induced Caᵥα₂δ-1 expression correlates with neuropathic pain development and maintenance, and various calcium channels are known to contribute to spinal synaptic neurotransmission and DRG neuron excitability, injury-induced Caᵥα₂δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα₂δ-1 subunit can block nerve injury-induced Caᵥα₂δ-1 upregulation and prevent the onset of allodynia and reserve established allodynia.

As mentioned above, the GO subunits of VGCC form the binding site for gabapentin and pregabalin, which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα₂δ subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009, supra; Vink and Alewood, 2012, supra, Zamponi et al., 2015, supra).

Thus, the present invention relates to compounds with inhibitory effect towards the α₂δ subunit, in particular the α₂δ-1 subunit, of voltage-gated calcium channels.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al., Nat Biotechnol 2009; 27: 659-666.).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al., Lancet Neurol. 2013 November; 12(11):1084-95.). In fact, positive synergistic interaction for several compounds, including analgesics, has been described (Schröder et al., J Pharmacol Exp Ther. 2011; 337:312-20. Erratum in: J Pharmacol Exp Ther. 2012; 342:232.; Zhang et al., Cell Death Dis. 2014; 5:e1138.; Gilron et al., 2013, supra).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious.

The major challenge to both drug combination strategies is the regulatory requirement for each individual drug to be shown to be safe as an individual agent and in combination (Hopkins, Nat Chem Biol. 2008; 4:682-90.).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008, supra).

Thus, in a preferred embodiment, the compounds of the present invention, having inhibitory effects towards the α₂δ subunit, in particular the α₂δ-1 subunit, of voltage-gated calcium channels, additionally inhibit mu opioid receptor. The present invention relates also to the advantages of having dual activity, for μ-receptor and the α₂δ-1 subunit of voltage-gated calcium channels, in the same molecule to treat chronic pain.

In this way, the present invention relates to compounds having a mechanism of action on blocking the α₂δ subunit, in particular the α₂δ-1 subunit, of voltage-gated calcium channels). The present invention also relates to compounds having a complementary dual mechanism of action (μ-receptor agonist and blocker of the β₂δ subunit, in particular the α₂δ-1 subunit, of voltage-gated calcium channels) which implies a better profile of tolerability than the strong opioids (morphine, oxycodone, fentanyl etc) and/or better efficacy and tolerability than gabapentinoids (pregabalin and gabapentin).

Pain is multimodal in nature, since in nearly all pain states several mediators, signaling pathways and molecular mechanisms are implicated. Consequently, monomodal therapies can be complemented with a dual mechanism of action to provide complete pain relief. Currently, combining existing therapies is a common clinical practice and many efforts are directed to assess the best combination of available drugs in clinical studies (Mao, J., Gold, M. S., Backonja, M.; 2011; J. Pain; 12; 157-166).

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

The authors of the present invention, have found a series of compounds that show pharmacological activity towards both the α₂δ subunit, in particular the α₂δ-1 subunit, of the voltage-gated calcium channel, or compounds that show dual pharmacological activity towards both the α₂δ subunit, in particular the α₂δ-1 subunit, of the voltage-gated calcium channel and the μ-opioid receptor (MOR or mu-opioid receptor) resulting in an innovative, effective, complementary and alternative solution for the treatment of pain.

In view of the existing results of the currently available therapies and clinical practices, the present invention offers a solution by developing compounds binding to a single target or by combining in a single compound binding to two different targets relevant for the treatment of pain. This was mainly achieved by providing the compounds according to the invention that bind to the α₂δ subunit, in particular the α₂β-1 subunit, of the voltage-gated calcium channel, or both to the μ-opioid receptor and to the α₂δ subunit, in particular the α₂δ-1 subunit, of the voltage-gated calcium channel.

### SUMMARY OF THE INVENTION

In this invention a family of structurally distinct pyrazole derivatives, encompassed by formula (I), which have a pharmacological activity towards the α₂δ subunit, in particular the α₂δ-1 subunit, of the voltage-gated calcium channel, or which have a dual pharmacological activity towards both the α₂δ subunit, in particular the β₂δ-1 subunit, of the voltage-gated calcium channel and the μ-opioid receptor, were identified thus solving the above problem of identifying alternative or improved pain treatments by offering such compounds.

The main object of the invention is directed to a compound having binding to the α₂δ subunit, in particular the α₂δ-1 subunit, of the voltage-gated calcium channel for use in the treatment of pain.

Another object of the invention is directed to a compound having a dual activity binding to the α₂δ subunit, in particular the α₂δ-1 subunit, of the voltage-gated calcium channel and the μ-opioid receptor for use in the treatment of pain.

As this invention is aimed at providing a compound or a chemically related series of compounds which act as ligands of the GO subunit, in particular the α₂δ-1 subunit, of the voltage-gated calcium channel and/or the μ-opioid receptor it is a very preferred embodiment if the compound has a binding expressed as Kᵢ responding to the following scales:

Kᵢ(μ) is preferably <1000 nM, more preferably <500 nM, even more preferably <100 nM.

Kᵢ(β₂δ-1) is preferably <10000 nM, more preferably <5000 nM, even more preferably <3000 nM or even more preferably <500 nM.

The invention is directed in a main aspect to a compound of general Formula (I)

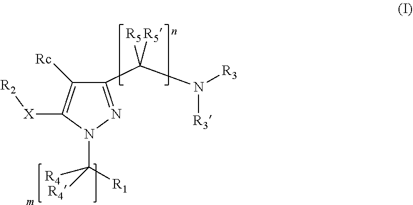

wherein R_{c}, R₁, R₂, R₃, R_{3′} R₄, R_{4′}, R₅, R_{5′}, X, m and n are as defined below in the detailed description.

A further object of the invention refers to the processes for preparation of compounds of general formula (I).

A still further object of the invention refers to the use of intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct pyrazole derivatives which have primary pharmacological activity towards the α₂δ subunit, in particular the α₂δ-1 subunit, of the voltage-gated calcium channel or which have a dual pharmacological activity towards both the α₂δ subunit, in particular the α₂δ-1 subunit, of the voltage-gated calcium channel and the μ-opioid receptor.

The invention is directed to compounds having primary activity binding to the α₂δ subunit, in particular the α₂δ-1 subunit, of the voltage-gated calcium channel or having a dual activity binding to the α₂δ subunit, in particular the α₂δ-1 subunit, of the voltage-gated calcium channel and the μ-opioid receptor for use in the treatment of pain.

As this invention is aimed at providing a compound or a chemically related series of compounds which act as ligands of the α₂δ subunit, in particular the α₂δ-1 subunit, of the voltage-gated calcium channel or as dual ligands of the α₂δ subunit, in particular the β₂δ-1 subunit, of the voltage-gated calcium channel and the μ-opioid receptor it is a preferred embodiment if the compound has a binding expressed as Kᵢ responding to the following scales:

Kᵢ(μ) is preferably <1000 nM, more preferably <500 nM, even more preferably <100 nM.

Kᵢ(α₂δ-1) is preferably <10000 nM, more preferably <5000 nM, even more preferably <3000 nM or even more preferably <500 nM.

The applicant has surprisingly found that the problem of providing a new effective and alternative for treating pain and pain related disorders can be solved by using an analgesic approach using binding to the α₂δ subunit, in particular the α₂δ-1 subunit, of the voltage-gated calcium channel or a multimodal balanced analgesic approach combining two different synergistic activities in a single drug (i.e., dual ligands which are bifunctional and bind to μ-opioid receptor and to α₂δ subunit, in particular the α₂δ-1 subunit, of the voltage-gated calcium channel), thereby enhancing through the α₂δ blockade without increasing the undesirable side effects of the μ-opioid activity. This supports the therapeutic value of a dual agent, whereby the α₂δ binding component acts as an intrinsic adjuvant of the MOR binding component.

A dual compound that possess binding to both the μ-opioid receptor and to the α₂δ subunit of the voltage-gated calcium channel shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the dual compounds according to the present invention show the following functionalities: blockade of the α₂δ subunit, in particular the α₂δ-1 subunit, of the voltage-gated calcium channel and μ-opioid receptor agonism.

It has to be noted, though, that functionalities “antagonism” and “agonism” are also sub-divided in their effect into subfunctionalities like partial agonism or inverse agonism. Accordingly, the functionalities of the compound should be considered within a relatively broad bandwidth.

An antagonist blocks or dampens agonist-mediated responses. Known subfunctionalities are neutral antagonists or inverse agonists.

An agonist increases the activity of the receptor above its basal level. Known subfunctionalities are full agonists, or partial agonists.

In addition, the two mechanisms complement each other since MOR agonists are only marginally effective in the treatment of neuropathic pain, while the blockers of the α₂δ subunit, in particular the α₂δ-1 subunit, of voltage-gated calcium channels show outstanding effects in preclinical neuropathic pain models. Thus, the α₂δ component, in particular the β₂δ-1 component, adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bornot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In its broader aspect, the present invention is directed to compounds of general Formula (I):

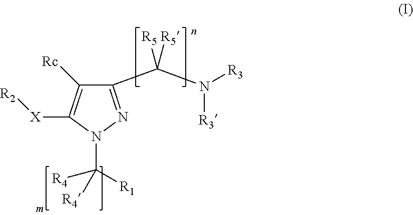

wherein

m is 0, 1, 2, 3 or 4;

n is 1, 2, 3 or 4;

X is C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;

R₂ is selected from substituted or unsubstituted aryl and substituted or unsubstituted heterocyclyl;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl,

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In its broader aspect, the present invention is directed to compounds of general Formula (I):

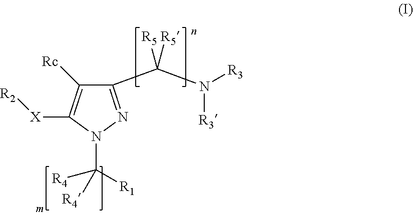

wherein

m is 0, 1, 2, 3 or 4;

n is 1, 2, 3 or 4;

X is C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted cycloalkyl and substituted or unsubstituted heterocyclyl;

R₂ is selected from substituted or unsubstituted aryl and substituted or unsubstituted heterocyclyl;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a particular embodiment the following compound is excluded:

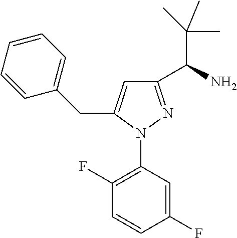 [SMILES: CC(C)(C)[C@@H](N)c1cc(Cc2ccccc2)n(-c2cc(F)ccc2F)n1]

In another particular embodiment the following proviso applies:

when X is —O—, then R₁ is selected from substituted or unsubstituted aryl and substituted or unsubstituted heterocyclyl;

In another particular embodiment the following proviso applies:

when X is —C(O)— and m is 0, then R₂ is selected from substituted or unsubstituted monocyclic aryl and substituted or unsubstituted monocyclic heterocyclyl;

In another particular embodiment the following proviso applies:

When X is —O—, then —[C(R₄R_{4′})]ₘ—R₁ is not unsubstituted methyl;

In another particular embodiment the following proviso applies:

when X is —C(O)— and m is 0, then R₂ is selected from substituted or unsubstituted monocyclic aryl and substituted or unsubstituted monocyclic aromatic heterocyclyl;

In another particular embodiment the following proviso applies:

When X is —CH₂—, then —[C(R₄R_{4′})]ₘ—R₁ is not unsubstituted methyl; In another particular embodiment the following proviso applies:

—[C(R₄R_{4′})]ₘ—R₁ is not unsubstituted methyl.

In a further embodiment the compound according to the invention of general Formula (I) is a compound of general Formula (I′)

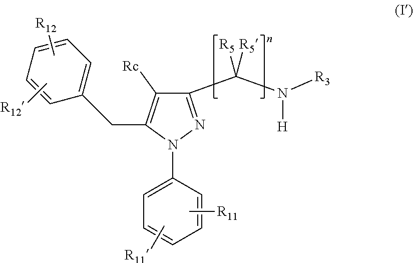

wherein R_{c}, R₅, R_{5′}, R₁₁, R_{11′}, R₁₂, R_{12′} and n are as defined in the description.

In a further embodiment the compound according to the invention of general Formula (I) is a compound of general Formula (I′), (I^{2′}), (I^{3′}), (I^{4′}), (I^{5′}), (I^{6′}), (I^{7′}), (I^{8′}), (I^{9′}), (I^{9a′}) or (I^{10′})

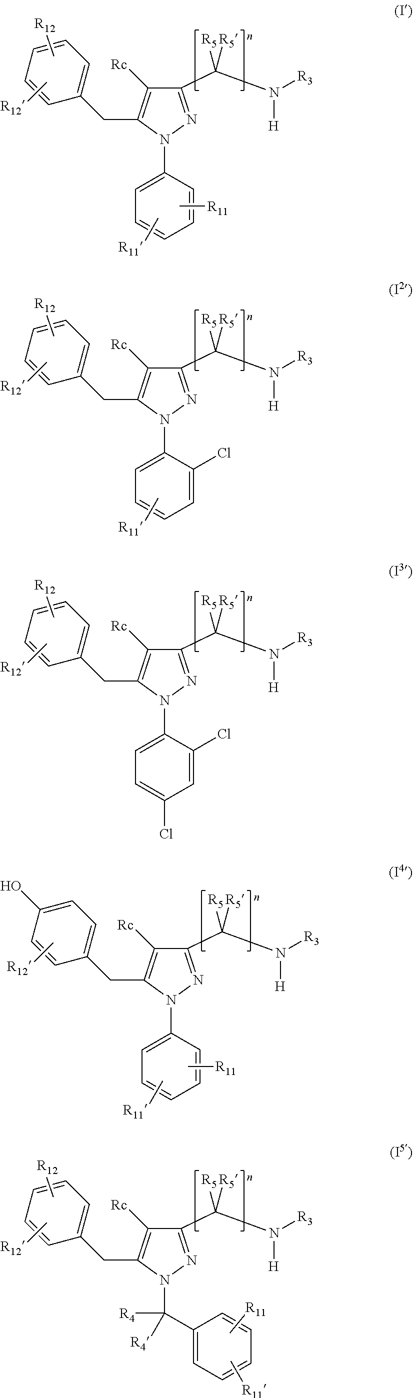

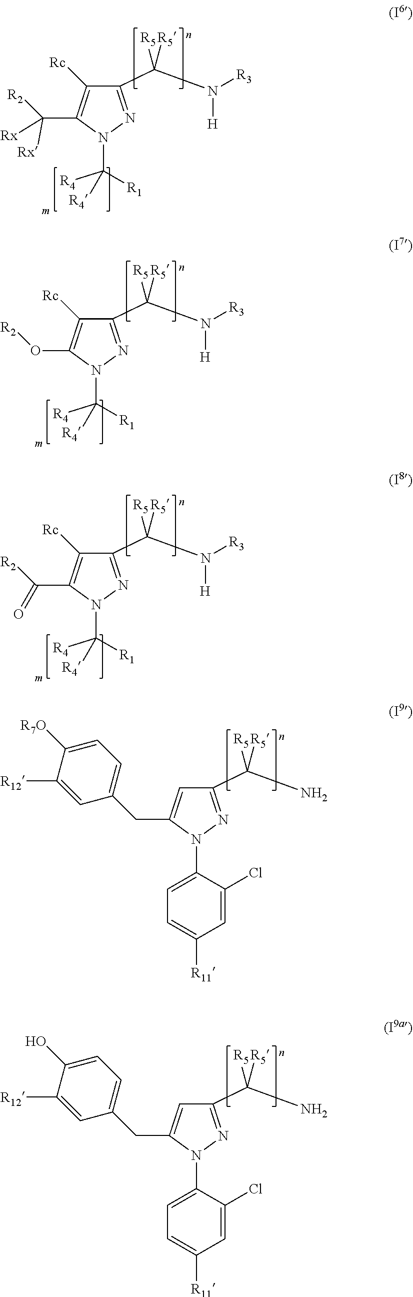

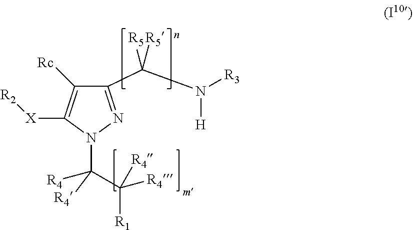

wherein R₁, R₂, R_{c}, R₄, R_{4′} R_{4″}, R_{4′″} R₅, R_{5′}, X, m, m′ and n are as defined in the description,
R₃ is independently selected from hydrogen, substituted or unsubstituted C₁₋₆ alkyl, substituted or unsubstituted C₂₋₆ alkenyl, substituted or unsubstituted C₂₋₆ alkynyl;
   wherein the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};
   wherein R₈ is selected from hydrogen, unsubstituted C₁₋₈ alkyl, unsubstituted C₂₋₈ alkenyl and unsubstituted C₂₋₈ alkynyl;
   and wherein R_{8′″} is selected from hydrogen, unsubstituted C₁₋₈ alkyl, unsubstituted C₂₋₈ alkenyl, unsubstituted C₂₋₈ alkynyl and -Boc;
R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6′″} and C(CH₃)₂OR₆;
   wherein R₆, R_{6′} and R_{6″} is selected from hydrogen, unsubstituted C₁₋₆ alkyl, unsubstituted C₂₋₆ alkenyl, unsubstituted C₂₋₆ alkynyl,
   and R_{6′″} is selected from hydrogen, unsubstituted C₁₋₆ alkyl, unsubstituted C₂₋₆ alkenyl, unsubstituted C₂₋₆ alkynyl and -Boc;
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;
   wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
   and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{2′})

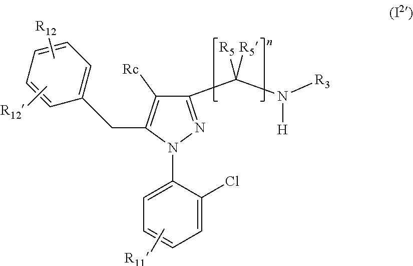

wherein R_{c}, R₅, R_{5′} and n are as defined in the description,

R₃ is selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;
R_{11′} is selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R₆, R_{6′} and R_{6″} is selected from hydrogen, unsubstituted C₁₋₆ alkyl, unsubstituted C₂₋₆ alkenyl, unsubstituted C₂₋₆ alkynyl,
and R_{6′″} is selected from hydrogen, unsubstituted C₁₋₆ alkyl, unsubstituted C₂₋₆ alkenyl, unsubstituted C₂₋₆ alkynyl and -Boc;
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;
R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{3′})

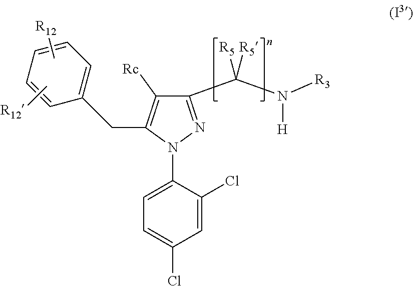

wherein R_{c}, R₅, R_{5′} and n are as defined in the description,

R₃ is independently selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR₇R_{7″}, and C(CH₃)₂OR₇;
R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{4′})

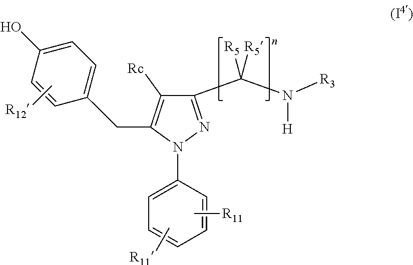

wherein R_{c}, R₅, R_{5′} and n are as defined in the description,

R₃ is independently selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;
R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6′″} and C(CH₃)₂OR₆;
R₆, R_{6′} and R_{6′″} is selected from hydrogen, unsubstituted C₁₋₆ alkyl, unsubstituted C₂₋₆ alkenyl, unsubstituted C₂₋₆ alkynyl,
and R_{6′″} is selected from hydrogen, unsubstituted C₁₋₆ alkyl, unsubstituted C₂₋₆ alkenyl, unsubstituted C₂₋₆ alkynyl and -Boc;
R_{12′} is selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;
R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{5′})

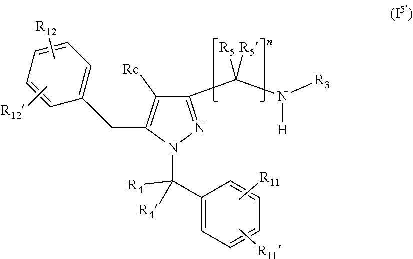

wherein R_{c}, R₄, R_{4′} R₅, R_{5′} and n are as defined in the description,

R₃ is independently selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;
R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R₆, R_{6′} and R_{6″} is selected from hydrogen, unsubstituted C₁₋₆ alkyl, unsubstituted C₂₋₆ alkenyl, unsubstituted C₂₋₆ alkynyl,
and R_{6′″} is selected from hydrogen, unsubstituted C₁₋₆ alkyl, unsubstituted C₂₋₆ alkenyl, unsubstituted C₂₋₆ alkynyl and -Boc;
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{6′})

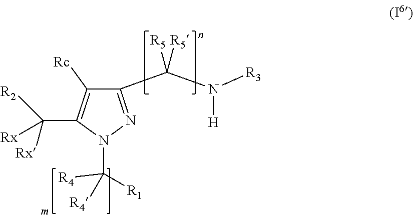

wherein R_{c}, R₁, R₂, R₃, R₄, R₄,_{′} R₅, R_{5′}, m, and n are as defined in the description.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{7′})

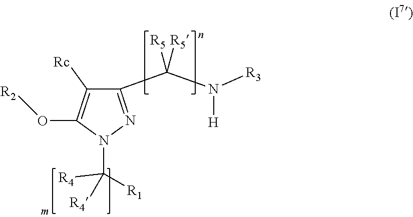

wherein R₁ is selected from substituted or unsubstituted aryl and substituted or unsubstituted heterocyclyl;

and wherein R_{c}, R₂, R₃, R₄, R₄,_{′} R₅, R_{5′}, m and n are as defined in the description.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{8′})

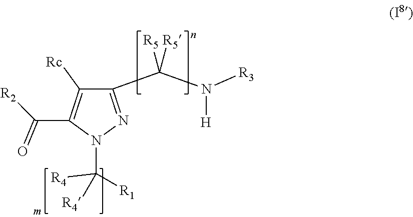

wherein R₂ is selected from substituted or unsubstituted monocyclic aryl and substituted or unsubstituted monocyclic aromatic heterocyclyl;

and wherein R_{c}, R₁, R₃, R₄, R₄,_{′} R₅, R_{5′}, m and n are as defined in the description.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{9′})

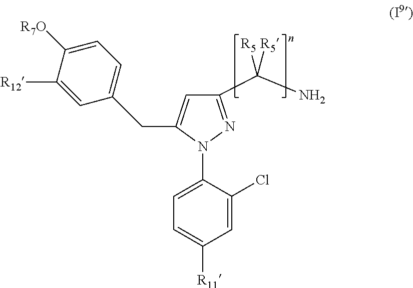

wherein R₇ is selected from hydrogen, unsubstituted C₁₋₆ alkyl, unsubstituted C₂₋₆ alkenyl and unsubstituted C₂₋₆ alkynyl;

R_{11′} is selected from hydrogen, halogen and —OR₆; preferably from halogen and —OR₆;

R_{12′} is selected from hydrogen, halogen and —OR₇; preferably from hydrogen and halogen;

and wherein R₅, R_{5′}, R₆, R₇, and n are as defined in the description.

In a further embodiment the compound according to the invention of general Formula (I) is a compound of general Formula (I^{9a′})

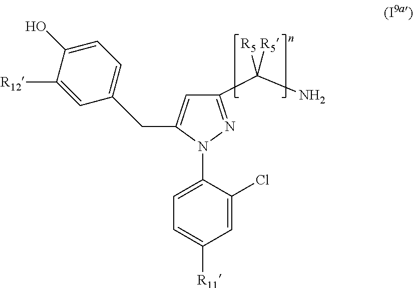

wherein R_{11′} is selected from hydrogen, halogen and —OR₆; and wherein R₅, R_{5′} and n are as defined in the description.

In a further embodiment the compound according to the invention of general Formula (I) is a compound of general Formula (I^{10′})

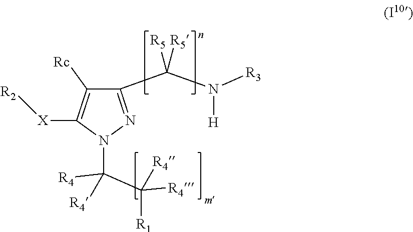

wherein R_{c}, R₁, R₂, R₃, R₄, R_{4′}, R₅, R_{5′}, n and X are as defined in the description. In addition, m′, R_{4″} and R_{4′″} are added. These are reflecting the statements below in the definitions of substitutions on alkyl etc. or aryl etc. that “when different radicals R₁ to R₁₂, and Rₓ and Rₓ′ are present simultaneously in Formula I they may be identical or different”. Thus this is reflecting that R_{4″} and R_{4′″} are or could be different from R₄ and R_{4′} or not and—accordingly—m′ being 0, 1, 2 or 3 is naturally resulting from m being 1, 2, 3 or 4.

In a further embodiment the compound according to the invention of general Formula (I)

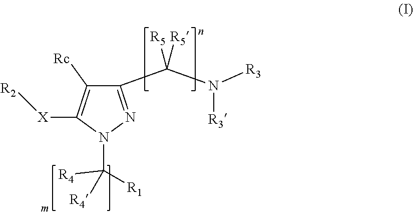

wherein

m is 0, 1, 2, 3 or 4;

n is 1, 2, 3 or 4;

X is —C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted aryl and substituted or unsubstituted heterocyclyl;

R₂ is selected from substituted or unsubstituted monocyclic aryl and substituted or unsubstituted monocyclic aromatic heterocyclyl;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl,

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I)

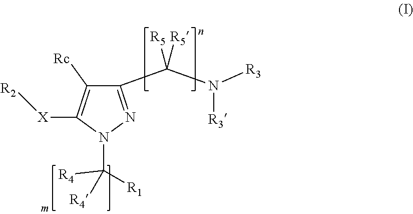

wherein

m is 0, 1, 2, 3 or 4;

n is 1, 2, 3 or 4;

X is —C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted aryl and substituted or unsubstituted heterocyclyl;
wherein the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6″} are independently selected from hydrogen, unsubstituted C₁₋₆ alkyl, unsubstituted C₂₋₆ alkenyl and unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

R₂ is selected from substituted or unsubstituted monocyclic aryl and substituted or unsubstituted monocyclic aromatic heterocyclyl; wherein said aryl or aromatic heterocyclyl in R₂, if substituted, is substituted
with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃ or R₃′, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, other than those defined in R₁, R₃ or R_{3′}, if substituted, is substituted with one or more substituent/s selected from —OR₉, halogen, —CN, haloalkyl, haloalkoxy and —NR₉R_{9′∝1};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I)

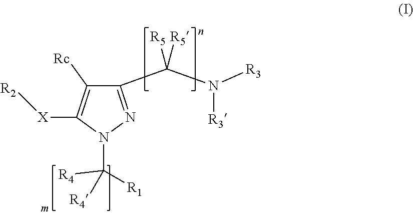

wherein

m is 0, 1, 2, 3 or 4;

n is 1, 2, 3 or 4;

X is —C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted cycloalkyl and substituted or unsubstituted heterocyclyl;
wherein the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};
wherein the cycloalkyl, aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6′″} are independently selected from hydrogen, unsubstituted C₁₋₆ alkyl, unsubstituted C₂₋₆ alkenyl and unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7′″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl, unsubstituted heterocyclyl, and -Boc;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃ or R_{3′}, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, other than those defined in R₁, R₃ or R_{3′}, if substituted, is substituted with one or more substituent/s selected from —OR₉, halogen, —CN, haloalkyl, haloalkoxy, unsubstituted heterocyclyl, —C(O)OR₉, —C(O)NR₉R_{9′″} and —NR₉R_{9′″};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

For clarity purposes, all groups and definitions described in the present description and referring to compounds of general Formula (I), also apply to compounds of general Formula Formulae (I′), (I^{2′}), (I^{3′}), (I^{4′}), (I^{5′}), (I^{6′}), (I^{7′}), (I^{8′}), (I^{9′}), (I^{9a′}) or (I^{10′}) (where applicable), as well as to all the intermediates of synthesis, when those groups are present in the mentioned general Markush formulae, since compounds of general Formulae (I′), (I^{2′}), (I^{3′}), (I^{4′}), (I^{5′}), (I^{6′}), (I^{7′}), (I^{8′}), (I^{9′}), (I^{9a′}) or (I^{10′}) are included within the scope of the larger definition of general Formula (I).

For clarity purposes, the general Markush Formula (I)

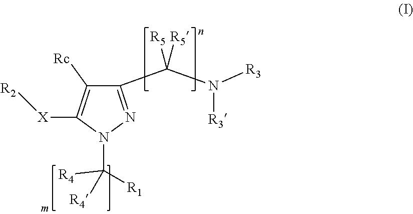

is equivalent to

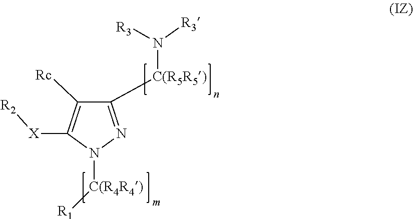

wherein only —C(R₄R_{4′})— or —C(R₅R_{5′})— are included into the brackets, and m or n means the number of times that —C(R₄R_{4′})— or —C(R₅R_{5′})— is repeated, respectively. The same would apply, when applicable, to general Markush Formulae (I′), (I^{2′}), (I^{3′}), (I^{4′}), (I^{5′}), (I^{6′}), (I^{7′}), (I^{8′}), (I^{9′}), (I^{9a′}) or (I^{10′}), and to all intermediates of synthesis.

In addition, and for clarity purposes, it should further be understood that naturally if m is 0, R₁ is still present when applicable in general (I), (I′), (I^{2′}), (I^{3′}), (I^{4′}), (I^{5′}), (I^{6′}), (I^{7′}), (I^{8′}), (I^{9′}), (I^{9a′}) or (I^{10′}), and to all intermediates of synthesis. In the same way when n is 0, —N(R₃R_{3′}) is still present, when applicable, in general (I), (I′), (I^{2′}), (I^{3′}), (I^{4′}), (I^{3′}), (I^{5′}), (I^{6′}), (I^{7′}), (I^{8′}), (I^{9′}), (I^{9a′}) or (I^{10′}), and to all intermediates of synthesis.

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—H₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NRₖR_{k∝″}, —SRₖ, —S(O)Rₖ, —S(O)₂Rₖ, —ORₖ, —C(O)ORₖ, —CN, —C(O)NRₖR_{k′}, haloalkyl, haloalkoxy or —OC₁₋₄alkyl, being Rₖ represented by R₆, R₈ or R₉, (being R_{k′} represented by R_{6′}, R_{8′} or R_{9′}; being R_{k″} represented by R_{6″}, R_{8″} or R_{9″}; being R_{k′″} represented by R_{6′″}, R_{8′″} or R_{9′″}), wherein R₁ to R_{12′} and Rₓ and R_{x′} are as defined in the description, and wherein when different radicals R₁ to R_{12′} and Rₓ and R_{x′} are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted is substituted with one or more of halogen (F, Cl, Br, I), —ORₖ, —CN, —SRₖ, —S(O)Rₖ, and —S(O)₂Rₖ, haloalkyl, haloalkoxy or —OC₁₋₄alkyl, being Rₖ represented by R₆, R₈ or R₉, (being R_{k′} represented by R_{6′}, R_{8′} or R_{9′}; being R_{k″} represented by R_{6″}, R_{8″} or R_{9″}; being R_{k′″} represented by R_{6′″}, R_{8′″} or R_{9′″}), wherein R₁ to R₁₂, and Rₓ and R_{x′} are as defined in the description, and wherein when different radicals R₁ to R_{12′} and Rₓ and R_{x′} are present simultaneously in Formula I they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 6 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic 5 to 18 membered ring contains one or more heteroatoms selected from the group consisting of nitrogen, oxygen and/or sulfur in the ring; preferably is an aromatic 5 to 18 membered mono or polycyclic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms selected from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, especially is benzodioxane, morpholine, tetrahydropyran, piperidine, oxopyrrolidine and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms selected from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning an cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 6 or 7 membered monocyclic aryl. Even more preferably the aryl is a 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —Rₖ, —ORₖ, —ON, —NO₂, —NRₖR_{k′″}, —C(O)ORₖ, NRₖC(O)R_{k′}, —C(O)NRₖR_{k′}, —NRₖS(O)₂R_{k′}, ═O, —OCH₂CH₂OH, —NRₖC(O)NR_{k′}R_{k″}, —S(O)₂NRₖR_{k′}, —NRₖS(O)₂NR_{k′}R_{k″}, haloalkyl, haloalkoxy, —SRₖ, —S(O)Rₖ, —S(O)₂Rₖ or —C(CH₃)ORₖ; NRₖR_{k′″}, with Rₖ and R_{k′″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆alkyl; a saturated or unsaturated, linear or branched, substituted or unsubstituted —C(O)—C₁₋₆-alkyl-group; a saturated or unsaturated, linear or branched, substituted or unsubstituted —C(O)—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being Rₖ one of R₆, R₇ or R₁₀, (being R_{k′} one of R_{6′}, R_{7′} or R_{10′}; being R_{k″} one of R_{6″}, R_{7″} or R_{11″}; being R_{k″″} one of R_{6′″}, R_{7′″} or R_{10′″}), wherein R₁ to R_{12′} and Rₓ and R_{x′} are as defined in the description, and wherein when different radicals R₁ to R_{12′} and Rₓ and R_{x′} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —Rk, —ORk, —CN, —NO₂, —NRₖR_{k′″}, NRₖC(O)R_{k′}, —NRₖS(O)₂R_{k′}, —S(O)₂NRₖR_{k′}, —NRₖC(O)NR_{k′}R_{k″}, haloalkyl, haloalkoxy, —SRₖ, —S(O)Rₖ or S(O)₂Rₖ; —OC₁₋₄alkyl being unsubstituted or substituted with one or more of ORₖ or halogen (F, Cl, I, Br), —CN, or —C₁₋₄alkyl, being Rₖ one of R₆, R₇ or R₁₀, (being R_{k′} one of R_{6′}, R_{7′} or R_{10″}; being R_{k″} one of R_{6″}, R_{7″} or R_{10″}; being R_{k″″} one of R_{6′″}, R_{7′″} or R_{10′″}), wherein R₁ to R₁₂, and Rₓ and R_{x′} are as defined in the description, and wherein when different radicals R₁ to R₁₂, and Rₓ and R_{x′} are present simultaneously in Formula I they may be identical or different.

In connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with ∇ (leading to a spiro structure) or with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl is spirosubstituted or substituted with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic—especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals.

Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is an N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a further embodiment the compound according to the invention of general Formula (I) is a compound wherein

m is 0, 1, 2, 3 or 4;

n is 1, 2, 3 or 4;

X is —C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;
wherein the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};
wherein the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6′″} are independently selected from hydrogen, unsubstituted C₁₋₆ alkyl, unsubstituted C₂₋₆ alkenyl, unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7′″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl,
wherein the alkyl, alkenyl or alkynyl in R₃ or R_{3′}, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, other than those defined in R₁, R₃ or R_{3′}, if substituted, is substituted with one or more substituent/s selected from —OR₉, halogen, —CN, haloalkyl, haloalkoxy and —NR₉R_{9′″};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

and wherein, the aryl, heterocyclyl or cycloalkyl other than those defined in R₁ or R₂, if substituted, is substituted with one or more substituent/s selected from halogen, —R₁₀, —OR₁₀, —NO₂, —NR₁₀R_{10′″}, NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —NR₁₀S(O)₂NR_{10′}R_{10″} and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{10′″} is selected from hydrogen, unsubstituted C₁₋₆ alkyl, unsubstituted C₂₋₆ alkenyl, unsubstituted C₂₋₆ alkynyl and -Boc;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2, 3 or 4;

n is 1, 2, 3 or 4;

X is —C(RₓR_{x′})—, —C(O)— or —O—;

R_{c} is selected from hydrogen, unsubstituted C₁₋₆ alkyl, unsubstituted C₂₋₆ alkenyl and unsubstituted C₂₋₆ alkynyl;

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;
wherein the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};
wherein the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6′″} are independently selected from hydrogen, unsubstituted C₁₋₆ alkyl, unsubstituted C₂₋₆ alkenyl, unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7′″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl,
wherein the alkyl, alkenyl or alkynyl in R₃ or R_{3′}, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, other than those defined in R₁, R₃ or R_{3′}, if substituted, is substituted with one or more substituent/s selected from —OR₉, halogen, —CN, haloalkyl, haloalkoxy and —NR₉R_{9′″};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2, 3 or 4;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 1, 2, 3 or 4;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —C(RₓR_{x′})—, —C(O)— or —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —C(RₓR_{x′})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —C(O)—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{c} is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{c} is selected from hydrogen or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{c} is selected from hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted cycloalkyl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted aryl, substituted or unsubstituted cycloalkyl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted aryl, substituted or unsubstituted cycloalkyl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is substituted or unsubstituted aryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from substituted or unsubstituted monocyclic aryl and substituted or unsubstituted monocyclic heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from substituted or unsubstituted monocyclic aryl and substituted or unsubstituted monocyclic aromatic heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from substituted or unsubstituted monocyclic aryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ and R_{3′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from hydrogen, substituted or unsubstituted C₁₋₆ alkyl, substituted or unsubstituted C₂₋₆ alkenyl, substituted or unsubstituted C₂₋₆ alkynyl,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{3′} is selected from hydrogen, substituted or unsubstituted C₁₋₆ alkyl, substituted or unsubstituted C₂₋₆ alkenyl, substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{3′} is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄ and R_{4′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅ and R_{5′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₆, R_{6′} and R_{6″} are independently selected from hydrogen, unsubstituted C₁₋₆ alkyl, unsubstituted C₂₋₆ alkenyl, unsubstituted C₂₋₆ alkynyl;

and R_{6′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₆, R_{6′} and R_{6′″} are independently selected from hydrogen, unsubstituted C₁₋₆ alkyl, unsubstituted C₂₋₆ alkenyl, unsubstituted C₂₋₆ alkynyl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₆, R_{6′} and R_{6′″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{6′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{6′″} is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{6′″} is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl, unsubstituted heterocyclyl, and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl, unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₇, R_{7′} and R_{7″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₇, R_{7′} and R_{7′″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
and wherein R_{7′″} is selected from hydrogen, unsubstituted C₁₋₆ alkyl, unsubstituted C₂₋₆ alkenyl, unsubstituted C₂₋₆ alkynyl, unsubstituted heterocyclyl, and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
and wherein R_{7′″} is selected from hydrogen, unsubstituted C₁₋₆ alkyl, unsubstituted C₂₋₆ alkenyl, unsubstituted C₂₋₆ alkynyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
and wherein R_{7′″} is unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₇, R_{7′} and R_{7″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₇, R_{7′} and R_{7″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
wherein R_{7′″} is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

wherein R₇ is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound of general Formula (I^{9′}) wherein

wherein R₇ is selected from hydrogen and unsubstituted C₁₋₆ alkyl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₈ is selected from hydrogen, unsubstituted C₁₋₈ alkyl, unsubstituted C₂₋₈ alkenyl and unsubstituted C₂₋₈ alkynyl;

and wherein R_{8′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₈ is selected from hydrogen, unsubstituted C₁₋₈ alkyl, unsubstituted C₂₋₈ alkenyl and unsubstituted C₂₋₈ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₈ is selected from hydrogen and unsubstituted C₁₋₈ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{8′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{8′″} is selected from hydrogen, unsubstituted C₁₋₈ alkyl, unsubstituted C₂₋₈ alkenyl and unsubstituted C₂₋₈ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{8′″} is selected from hydrogen and unsubstituted C₁₋₈ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₉ is selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₉ is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{9′″} is selected from hydrogen, unsubstituted C₁₋₈ alkyl, unsubstituted C₂₋₈ alkenyl and unsubstituted C₂₋₈ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{9′″} is selected from hydrogen and unsubstituted C₁₋₈ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{10′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₀, R_{10″} and R_{10′″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₀, R_{10′} and R_{10′″} are independently selected from hydrogen, unsubstituted C₁₋₆ alkyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{10′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{10′″} is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{10′″} is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR₆R₆ and C(CH₃)₂OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁ and R_{11′} are independently selected from hydrogen, halogen and —OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound general Formula (I^{9′}) wherein

R_{11′} is selected from hydrogen, halogen and —OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₂ and R_{12′} are independently selected from hydrogen, halogen, —OR₇, —NR₇R₇ and —NR₇S(O)₂R_{7′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₁₂ and R_{12′} are independently selected from hydrogen, halogen and —NR₇S(O)₂R_{7′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₓ and R_{x′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is —C(RₓR_{x′})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is —C(O)—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

m is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

m is 0 or 1 and n is 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

X is —C(RₓR_{x′})—, —C(O)— or —O—;

wherein

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0 or 1; and

n is 1 or 2; and

R_{c} is selected from hydrogen, unsubstituted C₁₋₆ alkyl, unsubstituted C₂₋₆ alkenyl and unsubstituted C₂₋₆ alkynyl; preferably R_{c} is hydrogen;

and

R₁ is selected from substituted or unsubstituted C₁₋₆ alkyl, substituted or unsubstituted aryl and substituted or unsubstituted heterocyclyl; preferably is selected from substituted or unsubstituted isobutyl, substituted or unsubstituted phenyl and substituted or unsubstituted pyridine;

and

R₂ is substituted or unsubstituted aryl, preferably is substituted or unsubstituted phenyl;

and

X is —C(RₓR_{x′})— or —O—; preferably —CH₂— or —O—;

and

R₃ is selected from hydrogen and substituted or unsubstituted C₁₋₆ alkyl, preferably from hydrogen, substituted or unsubstituted methyl and substituted or unsubstituted ethyl; more preferably from hydrogen, unsubstituted methyl and unsubstituted ethyl;

and

R_{3′} is selected from hydrogen and substituted or unsubstituted C₁₋₆ alkyl, preferably from hydrogen and substituted or unsubstituted methyl; more preferably from hydrogen and unsubstituted methyl;

and

R₄ and R_{4′} are independently selected from hydrogen and substituted or unsubstituted C₁₋₆ alkyl, preferably from hydrogen and substituted or unsubstituted methyl, more preferably from hydrogen and unsubstituted methyl;

and

R₅ and R_{5′} are independently selected from hydrogen and substituted or unsubstituted C₁₋₆ alkyl, preferably from hydrogen, substituted or unsubstituted methyl, substituted or unsubstituted ethyl and substituted or unsubstituted propyl, more preferably from hydrogen, unsubstituted methyl, unsubstituted ethyl and unsubstituted propyl;

and

Rₓ and R_{x′} are independently selected from hydrogen and substituted or unsubstituted C₁₋₆ alkyl; preferably from hydrogen and substituted or unsubstituted methyl, more preferably from hydrogen and unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 1, X is —C(RₓR_{x′})—, R_{c} is hydrogen, R_{3′} is hydrogen and R₂ is substituted or unsubstituted phenyl, preferably n is 1, m is 1, X is —CH₂—, R_{c} is hydrogen, R_{3′} is hydrogen and R₂ is substituted or unsubstituted phenyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 0, X is —C(RₓR_{x′})—, R_{c} is hydrogen, R_{3′} is hydrogen and R₂ is substituted or unsubstituted phenyl; preferably n is 1, m is 0, X is —CH₂—, R_{c} is hydrogen, R_{3′} is hydrogen and R₂ is substituted or unsubstituted phenyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 1, X is —C(RₓR_{x′})—, R₁ is selected from substituted or unsubstituted phenyl, substituted or unsubstituted pyridine and substituted or unsubstituted isobutyl, R_{c} is hydrogen and R_{3′} is hydrogen; preferably n is 1, m is 1, X is —CH₂—, R₁ is selected from substituted or unsubstituted phenyl, substituted or unsubstituted pyridine and substituted or unsubstituted isobutyl, R_{c} is hydrogen and R_{3′} is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 0, X is —C(RₓR_{x′})—, and R₁ is selected from substituted or unsubstituted phenyl, substituted or unsubstituted pyridine, substituted or unsubstituted isobutyl, R_{c} is hydrogen and R_{3′} is hydrogen; preferably n is 1, m is 0, X is —CH₂—, R₁ is selected from substituted or unsubstituted phenyl, substituted or unsubstituted pyridine and substituted or unsubstituted isobutyl, R_{c} is hydrogen and R_{3′} is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 1, X is —C(RₓR_{x′})—, and R₁ is selected from substituted or unsubstituted phenyl, substituted or unsubstituted pyridine, and substituted or unsubstituted isobutyl, R_{c} is hydrogen and R_{3′} is hydrogen; preferably n is 1, m is 1, X is —CH₂—, R₁ is selected from substituted or unsubstituted phenyl, substituted or unsubstituted pyridine and substituted or unsubstituted isobutyl, R_{c} is hydrogen and R_{3′} is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 1, X is —C(RₓR_{x′})—, R₁ is selected from substituted or unsubstituted phenyl, substituted or unsubstituted pyridine and substituted or unsubstituted isobutyl, R_{c} is hydrogen, R_{3′} is hydrogen and R₂ is substituted or unsubstituted phenyl; preferably n is 1, m is 1, X is —CH₂—, R₁ is selected from substituted or unsubstituted phenyl, substituted or unsubstituted pyridine and substituted or unsubstituted isobutyl, R_{c} is hydrogen, R_{3′} is hydrogen and R₂ is substituted or unsubstituted phenyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 0, X is —C(RₓR_{x′})—, R₁ is selected from substituted or unsubstituted phenyl, substituted or unsubstituted pyridine and substituted or unsubstituted isobutyl, R_{c} is hydrogen, R_{3′} is hydrogen and R₂ is substituted or unsubstituted phenyl; preferably n is 1, m is 0, X is —CH₂—, R₁ is selected from substituted or unsubstituted phenyl, substituted or unsubstituted pyridine and substituted or unsubstituted isobutyl, R_{c} is hydrogen, R_{3′} is hydrogen and R₂ is substituted or unsubstituted phenyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 0, X is —CH₂—, R₁ is phenyl substituted with chlorine in ortho position and R_{11′} in para position, R₂ is phenyl substituted with —OR₇ in para position, R_{c} is hydrogen, R_{3′} is hydrogen and R₃ is hydrogen; preferably n is 1, m is 0, X is —CH₂—, R₁ is phenyl substituted with chlorine in ortho position and with hydrogen, chlorine, fluorine or —OR₆ in para position, R₂ is phenyl substituted with hydroxyl, —O-methyl or —O-ethyl in para position, R_{c} is hydrogen, R_{3′} is hydrogen and R₃ is hydrogen; more preferably, n is 1, m is 0, X is —CH₂—, R₁ is phenyl substituted with chlorine in ortho position and with hydrogen, chlorine, fluorine, hydroxyl, —O-methyl or —O-ethyl in para position, R₂ is phenyl substituted with hydroxyl, —O-methyl or —O-ethyl in para position, R_{c} is hydrogen, R_{3′} is hydrogen and R₃ is hydrogen.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

n is 1, m is 0, X is —CH₂—, R₁ is phenyl substituted with chlorine in ortho position and R_{11′} in para position, R₂ is phenyl substituted with —OH in para position, R_{c} is hydrogen, R_{3′} is hydrogen and R₃ is hydrogen; preferably n is 1, m is 0, X is —CH₂—, R₁ is phenyl substituted with chlorine in ortho position and with hydrogen, chlorine, fluorine or —OR₆ in para position, R₂ is phenyl substituted with hydroxyl, —O-methyl or —O-ethyl in para position, R_{c} is hydrogen, R_{3′} is hydrogen and R₃ is hydrogen; more preferably, n is 1, m is 0, X is —CH₂—, R₁ is phenyl substituted with chlorine in ortho position and with hydrogen, chlorine, fluorine, hydroxyl, —O-methyl or —O-ethyl in para position, R₂ is phenyl substituted with hydroxyl, —O-methyl or —O-ethyl in para position, R_{c} is hydrogen, R_{3′} is hydrogen and R₃ is hydrogen.

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

m is 0, 1, 2, 3 or 4; preferably m is 0 or 1;

n is 1, 2, 3 or 4; preferably n is 1 or 2;

X is —C(RₓR_{x′})—, —C(O)— or —O—; preferably X is —C(RₓR_{x′})— or —O—;

and/or

R_{c} is selected from substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is isobutyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably is pyridine;
and/or

R₂ is selected from substituted or unsubstituted aryl and substituted or unsubstituted heterocyclyl;
wherein
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or

R₃ is selected from hydrogen, substituted or unsubstituted C₁₋₆ alkyl, substituted or unsubstituted C₂₋₆ alkenyl, substituted or unsubstituted C₂₋₆ alkynyl,

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne; and/or

R_{3′} is selected from hydrogen, substituted or unsubstituted C₁₋₆ alkyl, substituted or unsubstituted C₂₋₆ alkenyl, substituted or unsubstituted C₂₋₆ alkynyl,

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₄ and R_{4′} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or propyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₆, R_{6′} and R_{6′″} are independently selected from hydrogen, unsubstituted C₁₋₆ alkyl, unsubstituted C₂₋₆ alkenyl and unsubstituted C₂₋₆ alkynyl;

and R_{6′″} is selected from hydrogen, unsubstituted C₁₋₆ alkyl, unsubstituted C₂₋₆ alkenyl, unsubstituted C₂₋₆ alkynyl and -Boc;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₇, R_{7′} and R_{7″} are independently selected from hydrogen, unsubstituted C₁₋₆ alkyl, unsubstituted C₂₋₆ alkenyl and unsubstituted C₂₋₆ alkynyl;

and wherein R_{7′″} is selected from hydrogen, unsubstituted C₁₋₆ alkyl, unsubstituted C₂₋₆ alkenyl, unsubstituted C₂₋₆ alkynyl and -Boc;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₈ is selected from hydrogen, unsubstituted C₁₋₈ alkyl, unsubstituted C₂₋₈ alkenyl and unsubstituted C₂₋₈ alkynyl;

and wherein R_{8′″} is selected from hydrogen, unsubstituted C₁₋₈ alkyl, unsubstituted C₂₋₈ alkenyl, unsubstituted C₂₋₈ alkynyl and -Boc;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₉ is selected from hydrogen, unsubstituted C₁₋₆ alkyl, unsubstituted C₂₋₆ alkenyl, and unsubstituted C₂₋₆ alkynyl;

and wherein R_{9′″} is selected from hydrogen, unsubstituted C₁₋₈ alkyl, unsubstituted C₂₋₈ alkenyl, unsubstituted C₂₋₈ alkynyl and -Boc;

wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

Rₓ and R_{x′} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or

R₁₁ and R_{11′} are independently selected from hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR₆R₆ and C(CH₃)₂OR₆;

wherein
the alkyl is C₁₋₆ alkyl like methyl, ethyl, propyl, butyl, pentyl, hexyl, isopropyl, or 2-methylpropyl;
and/or
R₁₂ and R_{12′} are independently selected from hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;

wherein
the alkyl is C₁₋₆ alkyl like methyl, ethyl, propyl, butyl, pentyl, hexyl, isopropyl, or 2-methylpropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{c} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is isobutyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably is pyridine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is isobutyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably is pyridine or tetrahydropyrane;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; preferably the cycloalkyl is cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₓ and R_{x′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{3′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ and R_{4′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅ and R_{5′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or propyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆, R_{6′}, R_{6″} and R_{6′″} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇, R_{7′}, R_{7″} and R_{7′″} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇, R_{7′} and R_{7″} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{7′″} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is thiazole;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₈ and R_{8′″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₉ and R_{9′″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₀, R_{10′}, R_{10″} and R_{10″″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₁ and R_{11′} as defined in any of the embodiments of the present invention,
the alkyl is C₁₋₆ alkyl like methyl, ethyl, propyl, butyl, pentyl, hexyl, isopropyl, or 2-methylpropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₂ and R_{12′} as defined in any of the embodiments of the present invention,
the alkyl is C₁₋₆ alkyl like methyl, ethyl, propyl, butyl, pentyl, hexyl, isopropyl, or 2-methylpropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₓ and R_{x′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein R₁₁ and R_{11′} as defined in any of the embodiments of the present invention,
are present in ortho, meta or para position, preferably in ortho or para position; more preferably R₁₁ is in ortho position while R_{11′} is in para position; even more preferably R₁₁ is in ortho position;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein R₁₂ and R_{12′} as defined in any of the embodiments of the present invention,
are present in ortho, meta or para position, preferably in meta or para position; more preferably R₁₂ is in para position while R_{12′} is in meta position; even more preferably R₁₂ is in para position;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 1, 2, 3 or 4; preferably n is 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2, 3 or 4; preferably m is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is —C(RₓR_{x′})—, —C(O)— or —O—; preferably X is —C(RₓR_{x′})— or —O—; more preferably X is —CH₂— or —O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is —C(RₓR_{x′})—; preferably X is —CH₂—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0 or 1; and

n is 1 or 2; and

R_{c} is hydrogen;

and

R₁ is selected from substituted or unsubstituted isobutyl, substituted or unsubstituted phenyl, and substituted or unsubstituted pyridine;

and

R₂ is substituted or unsubstituted phenyl; and

X is —CH2- or —O—;

and

R₃ is selected from hydrogen, substituted or unsubstituted methyl and substituted or unsubstituted ethyl;

and

R_{3′} is selected from hydrogen and substituted or unsubstituted methyl;

and

R₄ and R_{4′} are both hydrogen;

and

R₅ and R_{5′} are both hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

R_{c} is hydrogen;

In a preferred embodiment

R₁ is substituted or unsubstituted isobutyl, substituted or unsubstituted phenyl, or substituted or unsubstituted pyridine;

In a preferred embodiment

R₁ is substituted or unsubstituted isobutyl, substituted or unsubstituted phenyl, substituted or unsubstituted pyridine, substituted or unsubstituted tetrahydropyrane or substituted or unsubstituted cyclohexyl;

In a preferred embodiment

R₁ is substituted or unsubstituted phenyl, substituted or unsubstituted tetrahydropyrane or substituted or unsubstituted cyclohexyl;

In a preferred embodiment

R₂ is a substituted or unsubstituted phenyl.

In a preferred embodiment

R₃ is hydrogen, substituted or unsubstituted methyl or substituted or unsubstituted ethyl; preferably hydrogen, unsubstituted methyl or unsubstituted ethyl.

In a preferred embodiment

R_{3′} is hydrogen or substituted or unsubstituted methyl; preferably hydrogen or unsubstituted methyl.

In a preferred embodiment

R₃ is hydrogen, substituted or unsubstituted methyl or substituted or unsubstituted ethyl; preferably hydrogen, unsubstituted methyl or unsubstituted ethyl, while R_{3′} is hydrogen or substituted or unsubstituted methyl; preferably hydrogen or unsubstituted methyl.

In a preferred embodiment

R₃ is hydrogen, substituted or unsubstituted methyl or substituted or unsubstituted ethyl; preferably hydrogen, unsubstituted methyl or unsubstituted ethyl, while R_{3′} is hydrogen.

In a preferred embodiment

R₃ is substituted or unsubstituted methyl or substituted or unsubstituted ethyl; preferably unsubstituted methyl or unsubstituted ethyl, while R_{3′} is hydrogen or substituted or unsubstituted methyl; preferably hydrogen or unsubstituted methyl.

In a preferred embodiment

R₃ is substituted or unsubstituted methyl; preferably unsubstituted methyl, while R_{3′} is substituted or unsubstituted methyl; preferably unsubstituted methyl.

In a preferred embodiment

R₃ is substituted or unsubstituted methyl; preferably unsubstituted methyl, while R_{3′} is hydrogen.

In a preferred embodiment

R₃ and R_{3′} are both hydrogen.

In a preferred embodiment

R₄ is hydrogen or substituted or unsubstituted methyl, preferably hydrogen or unsubstituted methyl.

In a preferred embodiment

R_{4′} is hydrogen.

In a preferred embodiment

R₄ is hydrogen or substituted or unsubstituted methyl, preferably hydrogen or unsubstituted methyl, while R_{4′} is hydrogen.

In a preferred embodiment

R₄ is substituted or unsubstituted methyl, preferably unsubstituted methyl, while R_{4′} is hydrogen.

In a preferred embodiment

R₄ and R_{4′} are both hydrogen.

In a preferred embodiment

R₅ is hydrogen, substituted or unsubstituted methyl, substituted or unsubstituted ethyl or substituted or unsubstituted propyl, preferably hydrogen, unsubstituted methyl, unsubstituted ethyl or unsubstituted propyl.

In a preferred embodiment

R_{5′} is hydrogen or substituted or unsubstituted methyl, preferably hydrogen unsubstituted methyl.

In a preferred embodiment

R₅ is hydrogen, substituted or unsubstituted methyl, substituted or unsubstituted ethyl or substituted or unsubstituted propyl, preferably hydrogen, unsubstituted methyl, unsubstituted ethyl or unsubstituted propyl, while R_{5′} is hydrogen or substituted or unsubstituted methyl, preferably hydrogen and unsubstituted methyl.

In a preferred embodiment

R₅ is hydrogen, substituted or unsubstituted methyl, substituted or unsubstituted ethyl or substituted or unsubstituted propyl, preferably hydrogen, unsubstituted methyl, unsubstituted ethyl or unsubstituted propyl, while R_{5′} is hydrogen.

In a preferred embodiment

R₅ and R_{5′} are both substituted or unsubstituted methyl, preferably R₅ and R_{5′} are both unsubstituted methyl.

In a preferred embodiment

R₅ and R_{5′} are both hydrogen.

In a preferred embodiment

R₅ is hydrogen, —CH₃, —CH₂CH₃, —CH₂CH₂CH₃, —CH₂OH, —CH₂-pyridine, —CH₂-morpholine, —CH₂C(O)OH or —CH₂C(O)NH₂.

In a preferred embodiment

R₅ is hydrogen, —CH₃, —CH₂CH₃, —CH₂CH₂CH₃, —CH₂OH, —CH₂-pyridine, —CH₂-morpholine, —CH₂C(O)OH or —CH₂C(O)NH₂, while R₅, is hydrogen or —CH₃.

In a preferred embodiment

R₆ is hydrogen, substituted or unsubstituted methyl or substituted or unsubstituted ethyl; preferably hydrogen, unsubstituted methyl or unsubstituted ethyl.

In a preferred embodiment

R₇ is hydrogen, substituted or unsubstituted methyl or substituted or unsubstituted ethyl; preferably hydrogen, unsubstituted methyl or unsubstituted ethyl.

In a preferred embodiment

R_{7′} is substituted or unsubstituted methyl; preferably unsubstituted methyl.

In a preferred embodiment

R₇ is hydrogen while R_{7′} is substituted or unsubstituted methyl; preferably R₇ is hydrogen while R_{7′} is unsubstituted methyl.

In a preferred embodiment

R₇ is hydrogen, —CH₃, —CH₂CH₃, —CH₂CH₂OH.

In a preferred embodiment

R_{7′} is —CH₃.

In a preferred embodiment

R_{7′″} is unsubstituted thiazole.

In a preferred embodiment

R₇ is hydrogen, while R_{7′} is —CH₃.

In a preferred embodiment

R₇ is hydrogen, while R_{7′″} is unsubstituted thiazole.

In a preferred embodiment

R₉ is hydrogen.

In a preferred embodiment

R_{9′″} is hydrogen.

In a preferred embodiment

R₉ and R_{9′″} are both hydrogen.

In a preferred embodiment

Rₓ is hydrogen or substituted or unsubstituted methyl, preferably hydrogen or unsubstituted methyl.

In a preferred embodiment

R_{x′} is hydrogen or substituted or unsubstituted methyl, preferably hydrogen or unsubstituted methyl.

In a preferred embodiment

Rₓ and R_{x′} are both substituted or unsubstituted methyl, preferably Rₓ and R_{x′} are both unsubstituted methyl.

In a preferred embodiment

Rₓ and R_{x′} are both hydrogen.

In a preferred embodiment

R₁₁ and R_{11′} are independently selected from hydrogen, chlorine, fluorine, hydroxy, substituted or unsubstituted —O-methyl and substituted or unsubstituted —O-ethyl, preferably hydrogen, chlorine, fluorine, hydroxy, unsubstituted —O-methyl and unsubstituted —O-ethyl.

In a preferred embodiment

R₁₁ is a group in ortho position selected from hydrogen, chlorine and substituted or unsubstituted —O-methyl, preferably is a group in ortho position selected from hydrogen, chlorine and unsubstituted —O-methyl.

In a preferred embodiment

R₁₁ is a group in meta position selected from hydrogen, hydroxy and substituted or unsubstituted —O-methyl, preferably is a group in meta position selected from hydrogen, hydroxy and unsubstituted —O-methyl.

In a preferred embodiment

R₁₁ is a group in para position selected from hydrogen, chlorine, fluorine, hydroxy, substituted or unsubstituted —O-methyl and substituted or unsubstituted —O-ethyl, preferably is a group in para position selected from hydrogen, chlorine, fluorine, hydroxy, unsubstituted —O-methyl and unsubstituted —O-ethyl.

In a preferred embodiment

R₁₁ is a group in ortho position selected from hydrogen, chlorine and substituted or unsubstituted —O-methyl, preferably is a group in ortho position selected from hydrogen, chlorine and unsubstituted —O-methyl, while R_{11′} is a group in para position selected from hydrogen, chlorine, fluorine, hydroxy, substituted or unsubstituted —O-methyl and substituted or unsubstituted —O— ethyl, preferably is a group in para position selected from hydrogen, chlorine, fluorine, hydroxy, unsubstituted —O-methyl and unsubstituted —O-ethyl.

In a preferred embodiment

R₁₁ is chlorine, in ortho position, while R_{11′} is substituted or unsubstituted —O— methyl, in meta position, preferably unsubstituted —O-methyl, in meta position.

In a preferred embodiment

R₁₁ is chlorine in ortho position, while R_{11′} is chlorine, in para position.

In a preferred embodiment

R₁₁ is hydrogen.

In a preferred embodiment

R_{11′} is hydrogen.

In a preferred embodiment

R₁₁ and R_{11′} are both hydrogen,

In a preferred embodiment

R₁₂ and R_{12′} are independently selected from hydrogen, fluorine, hydroxy, substituted or unsubstituted —O-methyl, substituted or unsubstituted —O-ethyl and —NHS(O)₂CH₃, preferably from hydrogen, fluorine, hydroxy, unsubstituted —O-methyl, unsubstituted —O-ethyl and —NHS(O)₂CH₃.

In a preferred embodiment

R₁₂ and R_{12′} are independently selected from hydrogen, fluorine, hydroxy, substituted or unsubstituted —O-methyl, substituted or unsubstituted —O-ethyl, —OCH₂CH₂OH, unsubstituted —NH-thiazole and —NHS(O)₂CH₃, preferably from hydrogen, fluorine, hydroxy, unsubstituted —O-methyl, unsubstituted —O-ethyl and —NHS(O)₂CH₃.

In a preferred embodiment

R₁₂ is a group in meta position selected from hydrogen, fluorine, hydroxy, substituted or unsubstituted —O-methyl, preferably is a group in meta position selected from hydrogen, fluorine, hydroxy or unsubstituted —O-methyl.

R₁₂ is a group in meta position selected from hydrogen, fluorine, hydroxy, substituted or unsubstituted —O-methyl, while R_{12′} is hydrogen; preferably is a group in meta position selected from hydrogen, fluorine, hydroxy or unsubstituted —O-methyl, while R_{12′} is hydrogen.

In a preferred embodiment

R₁₂ is a group in para position selected from hydrogen, hydroxy, substituted or unsubstituted —O-methyl, substituted or unsubstituted —O-ethyl and —NHS(O)₂CH₃, preferably is a group in para position selected from hydrogen, hydroxy, unsubstituted —O-methyl, unsubstituted —O-ethyl and —NHS(O)₂CH₃.

In a preferred embodiment

R₁₂ is a group in para position selected from hydrogen, hydroxy, substituted or unsubstituted —O-methyl, substituted or unsubstituted —O-ethyl, —OCH₂CH₂OH, unsubstituted —NH-thiazole and —NHS(O)₂CH₃, preferably is a group in para position selected from hydrogen, hydroxy, unsubstituted —O-methyl, unsubstituted —O-ethyl, —OCH₂CH₂OH, unsubstituted —NH-thiazole and —NHS(O)₂CH₃.

In a preferred embodiment

R₁₂ is a group in para position selected from hydrogen, hydroxy, substituted or unsubstituted —O-methyl, substituted or unsubstituted —O-ethyl and —NHS(O)₂CH₃, while R_{12′} is hydrogen; preferably R₁₂ is a group in para position selected from hydrogen, hydroxy, unsubstituted —O-methyl, unsubstituted —O— ethyl and —NHS(O)₂CH₃, while R_{12′} is hydrogen.

In a preferred embodiment

R₁₂ is a group in para position selected from hydrogen, hydroxy, substituted or unsubstituted —O-methyl, substituted or unsubstituted —O-ethyl, —OCH₂CH₂OH, unsubstituted —NH-thiazole and —NHS(O)₂CH₃, preferably is a group in para position selected from hydrogen, hydroxy, unsubstituted —O-methyl, unsubstituted —O-ethyl, —OCH₂CH₂OH, unsubstituted —NH-thiazole and —NHS(O)₂CH₃, while R_{12′} is hydrogen.

In a preferred embodiment

R₁₂ is hydroxy in para position, while R_{12′} is hydrogen.

In a preferred embodiment

R₁₂ is hydroxy in para position, while R_{12′} is fluorine.

In a preferred embodiment

R₁₂ is substituted or unsubstituted —O-methyl in para position, while R_{12′} is hydrogen; preferably R₁₂ is unsubstituted —O-methyl in para position, while R_{12′} is hydrogen.

In a preferred embodiment

R₁₂ is hydroxy, in para position, while R_{12′} is fluorine, in meta position.

In a preferred embodiment

R₁₂ is fluorine, in para position, while R_{12′} is hydroxy, in meta position.

In a preferred embodiment

R₁₂ is —OCH₂CH₂OH, in para position, while R_{12′} is hydrogen.

In a preferred embodiment

R₁₂ is—unsubstituted —NH-thiazole in para position, while R_{12′} is hydrogen.

In a preferred embodiment

R₁₂ is hydrogen.

In a preferred embodiment

R_{12′} is hydrogen.

In a preferred embodiment

R₁₂ and R_{12′} are both hydrogen.

In another preferred embodiment

n is 1.

In another preferred embodiment

n is 2.

In another preferred embodiment

m is 0.

In another preferred embodiment

m is 1.

In another preferred embodiment

X is —CH₂—.

In another preferred embodiment

X is —O—.

In an particular embodiment

the halogen is fluorine, chlorine, iodine or bromine.

In an particular embodiment

the halogen is fluorine or chlorine.

In a preferred further embodiment, the compounds of the general Formula (I) are selected from

| EX | Name |
|---|---|
| 1 | [1-(2,4-Dichlorophenyl)-5-(4-methoxybenzyl)-1H-pyrazol-3-yl]methanamine |
| 2 | [1-(2-Chlorophenyl)-5-(4-methoxybenzyl)-1H-pyrazol-3-yl]methanamine |
| 3 | [1-(4-Chlorophenyl)-5-(4-methoxybenzyl)-1H-pyrazol-3-yl]methanamine |
| 4 | [1-(2-Chloro-4-methoxyphenyl)-5-(4-methoxybenzyl)-1H-pyrazol-3-yl]methanamine |
| 5 | [1-(2-Chloro-4-fluorophenyl)-5-(4-methoxybenzyl)-1H-pyrazol-3-yl]methanamine |
| 6 | 4-{[3-(Aminomethyl)-1-(4-methoxyphenyl)-1H-pyrazol-5-yl]methyl}phenol |
| 7 | 4-{[3-(Aminomethyl)-1-(2-chloro-4-ethoxyphenyl)-1H-pyrazol-5-yl]methyl}phenol |
| 8 | [1-(2,4-Dichlorophenyl)-5-(3-methoxybenzyl)-1H-pyrazol-3-yl]methanamine |
| 9 | [5-Benzyl-1-(2,4-dichlorophenyl)-1H-pyrazol-3-yl]methanamine |
| 10 | [5-Benzyl-1-(4-methoxyphenyl)-1H-pyrazol-3-yl]methanamine |
| 11 | [1-Isobutyl-5-(4-methoxybenzyl)-1H-pyrazol-3-yl]methanamine |
| 12 | [1-(4-Chlorobenzyl)-5-(4-methoxybenzyl)-1H-pyrazol-3-yl]methanamine |
| 13 | [1-Benzyl-5-(4-methoxybenzyl)-1H-pyrazol-3-yl]methanamine |
| 14 | [1-(2-Chlorobenzyl)-5-(4-methoxybenzyl)-1H-pyrazol-3-yl]methanamine |
| 15 | [1-(2,4-Dichlorobenzyl)-5-(4-methoxybenzyl)-1H-pyrazol-3-yl]methanamine |
| 16 | [5-(4-Methoxybenzyl)-1-(1-phenylethyl)-1H-pyrazol-3-yl]methanamine |
| 17 | [1-(2,4-Dichlorophenyl)-5-(4-methoxyphenoxy)-1H-pyrazol-3- |
| | yl]methanamine |
| 18 | N-(4-{[3-(Aminomethyl)-1-(2,4-dichlorophenyl)-1H-pyrazol-5- |
| | yl]oxy}phenyl)methanesulfonamide |
| 19 | [5-(4-Methoxybenzyl)-1-phenyl-1H-pyrazol-3-yl]methanamine |
| 20 | [5-(4-Methoxybenzyl)-1-(pyridin-2-yl)-1H-pyrazol-3-yl]methanamine |
| 21 | 1-[1-(2,4-Dichlorophenyl)-5-(4-methoxybenzyl)-1H-pyrazol-3-yl]-N,N- |
| | dimethylmethanamine |
| 22 | 1-[1-(2,4-Dichlorophenyl)-5-(4-methoxybenzyl)-1H-pyrazol-3-yl]-N- |
| | methylmethanamine |
| 23 | N-{[1-Isobutyl-5-(4-methoxybenzyl)-1H-pyrazol-3-yl]methyl}ethanamine |
| 24 | 1-[1-(2,4-Dichlorophenyl)-5-(4-methoxybenzyl)-1H-pyrazol-3-yl]ethanamine |
| 25 | 1-[1-(2,4-Dichlorophenyl)-5-(4-ethoxybenzyl)-1H-pyrazol-3-yl]ethanamine |
| 26 | N-(4-{[3-(1-Aminoethyl)-1-(2,4-dichlorophenyl)-1H-pyrazol-5- |
| | yl]methyl}phenyl)methanesulfonamide |
| 27 | 4-{[3-(1-Aminoethyl)-1-(2-chloro-4-methoxyphenyl)-1H-pyrazol-5- |
| | yl]methyl}phenol |
| 28 | 4-{[3-(1-Aminoethyl)-1-(2-chloro-5-methoxyphenyl)-1H-pyrazol-5- |
| | yl]methyl}phenol |
| 29 | 4-{[3-(1-Aminoethyl)-1-(4-chloro-2-methoxyphenyl)-1H-pyrazol-5- |
| | yl]methyl}phenol |
| 30 | 1-[1-(2,4-Dichlorophenyl)-5-(3-fluoro-4-methoxybenzyl)-1H-pyrazol-3- |
| | yl)]ethanamine |
| 31 | 1-[1-(2,4-Dichlorophenyl)-5-(3-fluoro-4-methoxybenzyl)-1H-pyrazol-3- |
| | yl]butan-1-amine |
| 32 | 1-[1-(2,4-Dichlorophenyl)-5-(4-methoxybenzyl)-1H-pyrazol-3-yl]propan- |
| | 1-amine |
| 33 | 1-[1-(2,4-Dichlorophenyl)-5-(2-(4-methoxyphenyl)propan-2-yl)-1H- |
| | pyrazol-3-yl]ethanamine |
| 34 | 3-{[3-(Aminomethyl)-1-(2,4-dichlorophenyl)-1H-pyrazol-5-yl]methyl}phenol |
| 35 | 4-[3-(Aminomethyl)-5-benzyl-1H-pyrazol-1-yl]phenol |
| 36 | 3-[3-(Aminomethyl)-5-benzyl-1H-pyrazol-1-yl]phenol |
| 37 | 4-{[3-(Aminomethyl)-1-(2-chloro-4-methoxyphenyl)-1H-pyrazol-5- |
| | yl]methyl}phenol |
| 38 | 4-{[3-(Aminomethyl)-1-(2,4-dichlorophenyl)-1H-pyrazol-5-yl]methyl}phenol |
| 39 | 4-{[3-(Aminomethyl)-1-(2-chlorophenyl)-1H-pyrazol-5-yl]methyl}phenol |
| 40 | 4-{[3-(Aminomethyl)-1-(4-chlorophenyl)-1H-pyrazol-5-yl]methyl}phenol |
| 41 | 4-[3-(Aminomethyl)-5-(4-hydroxybenzyl)-1H-pyrazol-1-yl]-3-chlorophenol |
| 42 | 4-{[3-(Aminomethyl)-1-(2-chloro-4-fluorophenyl)-1H-pyrazol-5- |
| | yl]methyl}phenol |
| 43 | 4-{[3-(Aminomethyl)-1-(pyridin-2-yl)-1H-pyrazol-5-yl]methyl}phenol |
| 44 | 4-{[3-(Aminomethyl)-1-isobutyl-1H-pyrazol-5-yl]methyl}phenol |
| 45 | 4-{[3-(Aminomethyl)-1-(4-chlorobenzyl)-1H-pyrazol-5-yl]methyl}phenol |
| 46 | 4-{[3-(Aminomethyl)-1-benzyl-1H-pyrazol-5-yl]methyl}phenol |
| 47 | 4-{[3-(Aminomethyl)-1-(2-chlorobenzyl)-1H-pyrazol-5-yl]methyl}phenol |
| 48 | 4-{[3-(Aminomethyl)-1-(2,4-dichlorobenzyl)-1H-pyrazol-5-yl]methyl}phenol |
| 49 | 4-{[3-(Aminomethyl)-1-(1-phenylethyl)-1H-pyrazol-5-yl]methyl}phenol |
| 50 | 4-({3-[(ethylamino)methyl]-1-isobutyl-1H-pyrazol-5-yl}methyl)phenol |
| 51 | 4-{[3-(1-Aminoethyl)-1-(2,4-dichlorophenyl)-1H-pyrazol-5-yl]methyl}phenol |
| 52 | 4-{[3-(1-Aminoethyl)-1-(2,4-dichlorophenyl)-1H-pyrazol-5-yl]methyl}-2- |
| | fluorophenol |
| 53 | 4-{[3-(1-Aminobutyl)-1-(2,4-dichlorophenyl)-1H-pyrazol-5-yl]methyl}-2- |
| | fluorophenol |
| 54 | 4-{[3-(1-Aminopropyl)-1-(2,4-dichlorophenyl)-1H-pyrazol-5-yl]methyl}phenol |
| 55 | 4-{2-[3-(1-Aminoethyl)-1-(2,4-dichlorophenyl)-1H-pyrazol-5-yl]propan-2- |
| | yl}phenol |
| 56 | 4-{[3-(2-Aminopropan-2-yl)-1-(2,4-dichlorophenyl)-1H-pyrazol-5-yl]methyl}- |
| | 2-fluorophenol |
| 57 | 4-{[3-(2-Aminopropan-2-yl)-1-(2,4-dichlorophenyl)-1H-pyrazol-5- |
| | yl]methyl}phenol |
| 58 | 4-{[3-(2-Aminoethyl)-1-(2,4-dichlorophenyl)-1H-pyrazol-5-yl]methyl}phenol |
| 59 | 4-{[3-(1-Aminopropan-2-yl)-1-(2,4-dichlorophenyl)-1H-pyrazol-5- |
| | yl]methyl}phenol |
| 60 | 4-{[3-(1-Aminobutan-2-yl)-1-(2,4-dichlorophenyl)-1H-pyrazol-5- |
| | yl]methyl}phenol |
| 61 | (S)-4-{[3-(1-Aminoethyl)-1-(2,4-dichlorophenyl)-1H-pyrazol-5- |
| | yl]methyl}phenol |
| 62 | (R)-4-{[3-(1-Aminoethyl)-1-(2,4-dichlorophenyl)-1H-pyrazol-5- |
| | yl]methyl}phenol |
| 63 | (S)-4-{[3-(1-Aminoethyl)-1-(2,4-dichlorophenyl)-1H-pyrazol-5-yl]methyl}-2- |
| | fluorophenol |
| 64 | (R)-4-{[3-(1-Aminoethyl)-1-(2,4-dichlorophenyl)-1H-pyrazol-5-yl]methyl}-2- |
| | fluorophenol |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | Structure | Chemical name |
|---|---|---|
| 1 | 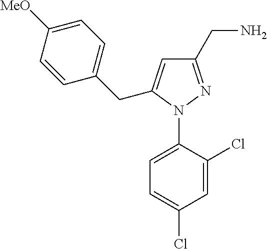 [SMILES: COc1ccc(Cc2cc(CN)nn2-c2ccc(Cl)cc2Cl)cc1] | [1-(2,4-Dichlorophenyl)-5-(4-methoxybenzyl)-1H- pyrazol-3 yl]methanamine |
| 2 | 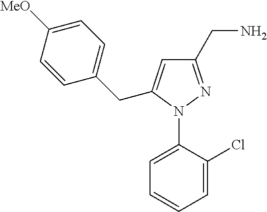 [SMILES: COc1ccc(Cc2cc(CN)nn2-c2ccccc2Cl)cc1] | [1-(2-Chlorophenyl)-5-(4-methoxybenzyl)-1H-pyrazol- 3-yl]methanamine |
| 3 | 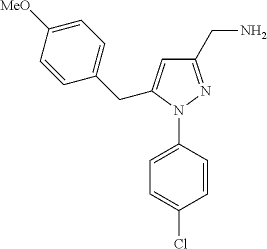 [SMILES: COc1ccc(Cc2cc(CN)nn2-c2ccc(Cl)cc2)cc1] | [1-(4-Chlorophenyl)-5-(4-methoxybenzyl)-1H-pyrazol- 3-yl]methanamine |
| 4 | 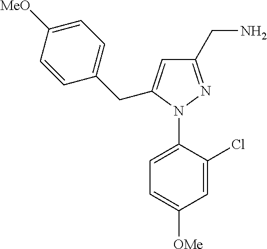 [SMILES: COc1ccc(Cc2cc(CN)nn2-c2ccc(OC)cc2Cl)cc1] | [1-(2-Chloro-4-methoxyphenyl)-5-(4-methoxybenzyl)- 1H-pyrazol-3-yl]methanamine |
| 5 | 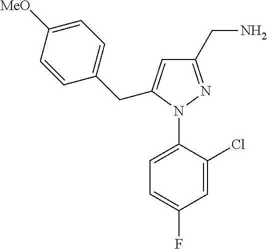 [SMILES: COc1ccc(Cc2cc(CN)nn2-c2ccc(F)cc2Cl)cc1] | [1-(2-Chloro-4-fluorophenyl)-5-(4-methoxybenzyl)- 1H-pyrazol-3-yl]methanamine |
| 6 | 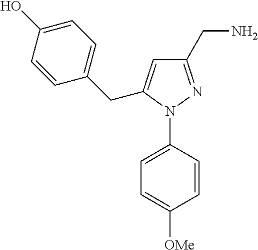 [SMILES: COc1ccc(-n2nc(CN)cc2Cc2ccc(O)cc2)cc1] | 4-{[3-(Aminomethyl)-1-(4-methoxyphenyl)-1H- pyrazol-5-yl]methyl}phenol |
| 7 | 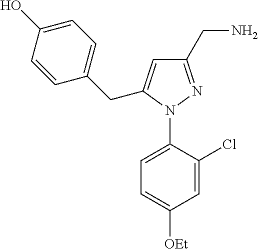 [SMILES: CCOc1ccc(-n2nc(CN)cc2Cc2ccc(O)cc2)c(Cl)c1] | 4-{[3-(Aminomethyl)-1-(2-chloro-4-ethoxyphenyl)-1H- pyrazol-5-yl]methyl}phenol |
| 8 | 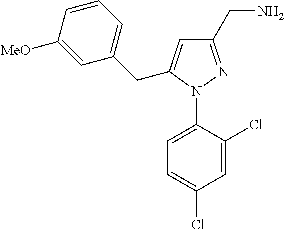 [SMILES: COc1cccc(Cc2cc(CN)nn2-c2ccc(Cl)cc2Cl)c1] | [1-(2,4-Dichlorophenyl)-5-(3-methoxybenzyl)-1H- pyrazol-3-yl]methanamine |
| 9 | 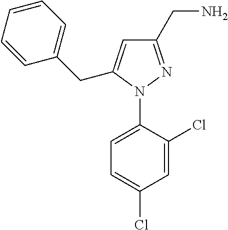 [SMILES: NCc1cc(Cc2ccccc2)n(-c2ccc(Cl)cc2Cl)n1] | [5-Benzyl-1-(2,4-dichlorophenyl)-1H-pyrazol-3- yl]methanamine |
| 10 | 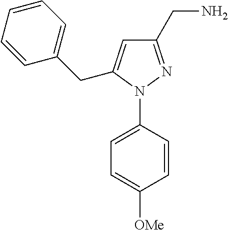 [SMILES: COc1ccc(-n2nc(CN)cc2Cc2ccccc2)cc1] | [5-Benzyl-1-(4-methoxyphenyl)-1H-pyrazol-3- yl]methanamine |
| 11 | 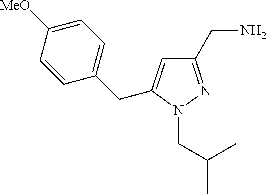 [SMILES: COc1ccc(Cc2cc(CN)nn2CC(C)C)cc1] | [1-Isobutyl-5-(4-methoxybenzyl)-1H-pyrazol-3- yl]methanamine |
| 12 | 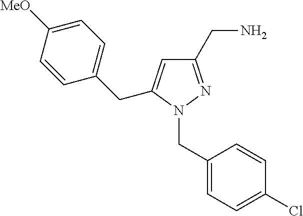 [SMILES: COc1ccc(Cc2cc(CN)nn2Cc2ccc(Cl)cc2)cc1] | [1-(4-Chlorobenzyl)-5-(4-methoxybenzyl)-1H-pyrazol- 3-yl]methanamine |
| 13 | 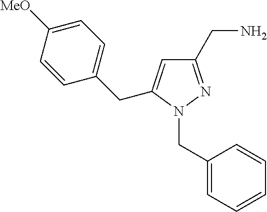 [SMILES: COc1ccc(Cc2cc(CN)nn2Cc2ccccc2)cc1] | [1-Benzyl-5-(4-methoxybenzyl)-1H-pyrazol-3- yl]methanamine |
| 14 | 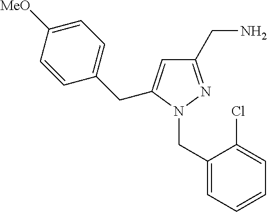 [SMILES: COc1ccc(Cc2cc(CN)nn2Cc2ccccc2Cl)cc1] | [1-(2-Chlorobenzyl)-5-(4-methoxybenzyl)-1H-pyrazol- 3-yl]methanamine |
| 15 | 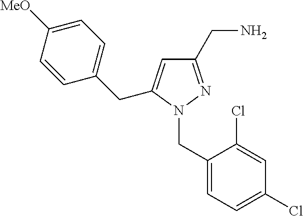 [SMILES: COc1ccc(Cc2cc(CN)nn2Cc2ccc(Cl)cc2Cl)cc1] | [1-(2,4-Dichlorobenzyl)-5-(4-methoxybenzyl)-1H- pyrazol-3-yl]methanamine |
| 16 | 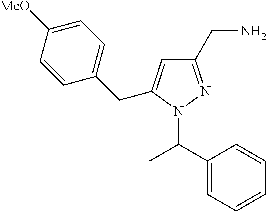 [SMILES: COc1ccc(Cc2cc(CN)nn2C(C)c2ccccc2)cc1] | [5-(4-Methoxybenzyl)-1-(1-phenylethyl)-1H-pyrazol-3- yl]methanamine |
| 17 | 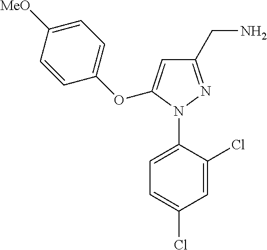 [SMILES: COc1ccc(Oc2cc(CN)nn2-c2ccc(Cl)cc2Cl)cc1] | [1-(2,4-Dichlorophenyl)-5-(4-methoxyphenoxy)-1H- pyrazol-3-yl]methanamine |
| 18 | 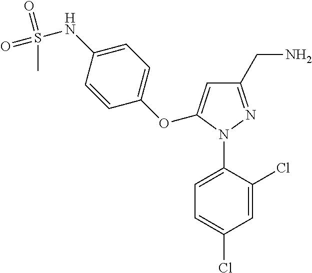 [SMILES: CS(=O)(=O)Nc1ccc(Oc2cc(CN)nn2-c2ccc(Cl)cc2Cl)cc1] | N-(4-{[3-(Aminomethyl)-1-(2,4-dichlorophenyl)-1H- pyrazol-5-yl]oxy}phenyl)methanesulfonamide |
| 19 | 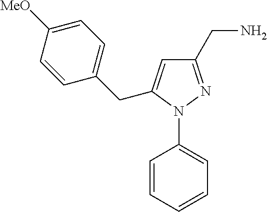 [SMILES: COc1ccc(Cc2cc(CN)nn2-c2ccccc2)cc1] | [5-(4-Methoxybenzyl)-1-phenyl-1H-pyrazol-3- yl]methanamine |
| 20 | 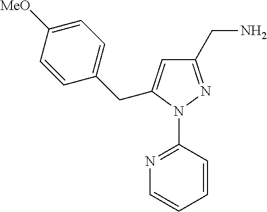 [SMILES: COc1ccc(Cc2cc(CN)nn2-c2ccccn2)cc1] | Example 20. [5-(4-Methoxybenzyl)-1-(pyridin-2-yl)- 1H-pyrazol-3-yl]methanamine |
| 21 | 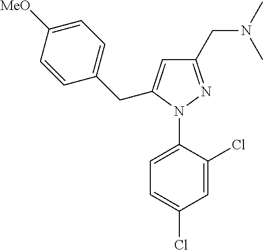 [SMILES: COc1ccc(Cc2cc(CN(C)C)nn2-c2ccc(Cl)cc2Cl)cc1] | 1-[1-(2,4-Dichlorophenyl)-5-(4-methoxybenzyl)-1H- pyrazol-3-yl]-N,N-dimethylmethanamine |
| 22 | 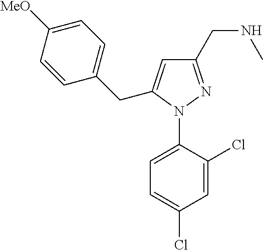 [SMILES: CNCc1cc(Cc2ccc(OC)cc2)n(-c2ccc(Cl)cc2Cl)n1] | 1-[1-(2,4-Dichlorophenyl)-5-(4-methoxybenzyl)-1H- pyrazol-3-yl]-N-methylmethanamine |
| 23 | 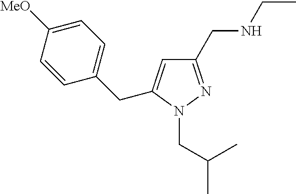 [SMILES: CCNCc1cc(Cc2ccc(OC)cc2)n(CC(C)C)n1] | N-{[1-Isobutyl-5-(4-methoxybenzyl)-1H-pyrazol-3- yl]methyl}ethanamine |
| 24 | 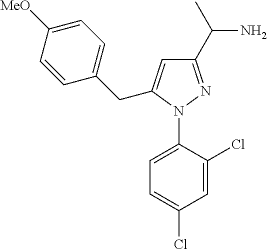 [SMILES: COc1ccc(Cc2cc(C(C)N)nn2-c2ccc(Cl)cc2Cl)cc1] | 1-[1-(2,4-Dichlorophenyl)-5-(4-methoxybenzyl)-1H- pyrazol-3-yl]ethanamine |
| 25 | 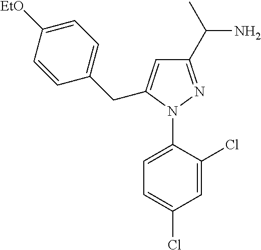 [SMILES: CCOc1ccc(Cc2cc(C(C)N)nn2-c2ccc(Cl)cc2Cl)cc1] | 1-[1-(2,4-Dichlorophenyl)-5-(4-ethoxybenzyl)-1H- pyrazol-3-yl]ethanamine |
| 26 | 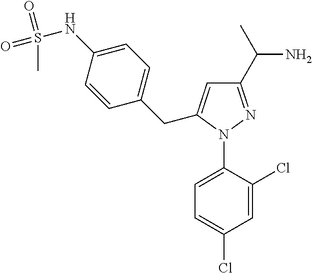 [SMILES: CC(N)c1cc(Cc2ccc(NS(C)(=O)=O)cc2)n(-c2ccc(Cl)cc2Cl)n1] | N-(4-{[3-(1-Aminoethyl)-1-(2,4-dichlorophenyl)-1H- pyrazol-5-yl]methyl}phenyl)methanesulfonamide |
| 27 | 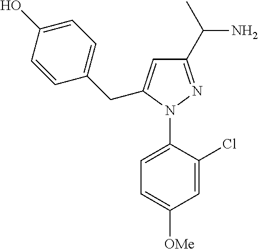 [SMILES: COc1ccc(-n2nc(C(C)N)cc2Cc2ccc(O)cc2)c(Cl)c1] | 4-{[3-(1-Aminoethyl)-1-(2-chloro-4-methoxyphenyl)- 1H-pyrazol-5-yl]methyl}phenol |
| 28 | 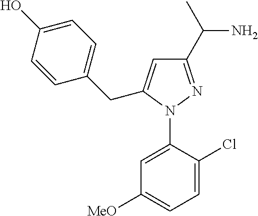 [SMILES: COc1ccc(Cl)c(-n2nc(C(C)N)cc2Cc2ccc(O)cc2)c1] | 4-{[3-(1-Aminoethyl)-1-(2-chloro-5-methoxyphenyl)- 1H-pyrazol-5-yl]methyl}phenol |
| 29 | 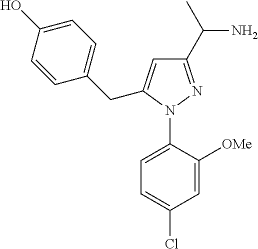 [SMILES: COc1cc(Cl)ccc1-n1nc(C(C)N)cc1Cc1ccc(O)cc1] | 4-{[3-(1-Aminoethyl)-1-(4-chloro-2-methoxyphenyl)- 1H-pyrazol-5-yl]methyl}phenol |
| 30 | 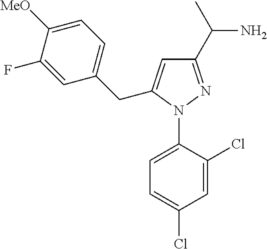 [SMILES: COc1ccc(Cc2cc(C(C)N)nn2-c2ccc(Cl)cc2Cl)cc1F] | 1-[1-(2,4-Dichlorophenyl)-5-(3-fluoro-4- methoxybenzyl)-1H-pyrazol-3-yl)]ethanamine |
| 31 | 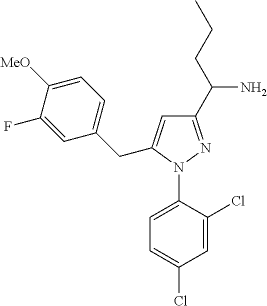 [SMILES: CCCC(N)c1cc(Cc2ccc(OC)c(F)c2)n(-c2ccc(Cl)cc2Cl)n1] | 1-[1-(2,4-Dichlorophenyl)-5-(3-fluoro-4- methoxybenzyl)-1H-pyrazol-3-yl]butan-1-amine |
| 32 | 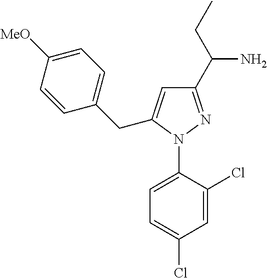 [SMILES: CCC(N)c1cc(Cc2ccc(OC)cc2)n(-c2ccc(Cl)cc2Cl)n1] | 1-[1-(2,4-Dichlorophenyl)-5-(4-methoxybenzyl)-1H- pyrazol-3-yl]propan-1-amine |
| 33 | 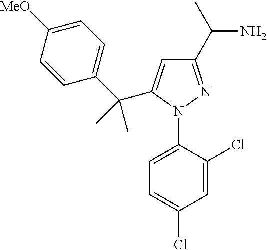 [SMILES: COc1ccc(C(C)(C)c2cc(C(C)N)nn2-c2ccc(Cl)cc2Cl)cc1] | 1-[1-(2,4-Dichlorophenyl)-5-(2-(4- methoxyphenyl)propan-2-yl)-1H-pyrazol-3- yl]ethanamine |
| 34 | 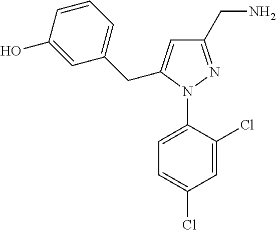 [SMILES: NCc1cc(Cc2cccc(O)c2)n(-c2ccc(Cl)cc2Cl)n1] | . 3-{[3-(Aminomethyl)-1-(2,4-dichlorophenyl)-1H- pyrazol-5-yl]methyl}phenol |
| 35 | 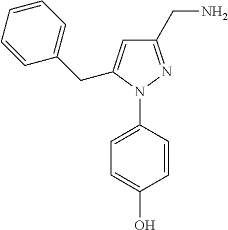 [SMILES: NCc1cc(Cc2ccccc2)n(-c2ccc(O)cc2)n1] | 4-[3-(Aminomethyl)-5-benzyl-1H-pyrazol-1-yl]phenol |
| 36 | 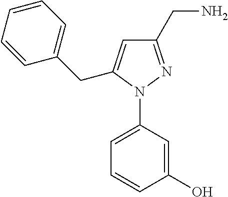 [SMILES: NCc1cc(Cc2ccccc2)n(-c2cccc(O)c2)n1] | 3-[3-(Aminomethyl)-5-benzyl-1H-pyrazol-1-yl]phenol |
| 37 | 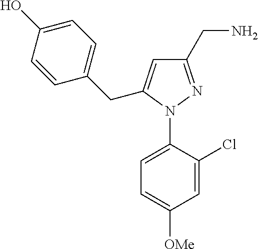 [SMILES: COc1ccc(-n2nc(CN)cc2Cc2ccc(O)cc2)c(Cl)c1] | 4-{[3-(Aminomethyl)-1-(2-chloro-4-methoxyphenyl)- 1H-pyrazol-5-yl]methyl}phenol |
| 38 | 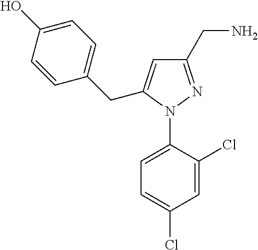 [SMILES: NCc1cc(Cc2ccc(O)cc2)n(-c2ccc(Cl)cc2Cl)n1] | 4-{[3-(Aminomethyl)-1-(2,4-dichlorophenyl)-1H- pyrazol-5-yl]methyl}phenol |
| 39 | 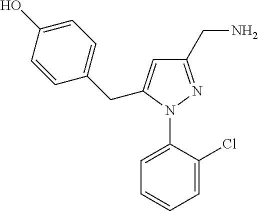 [SMILES: NCc1cc(Cc2ccc(O)cc2)n(-c2ccccc2Cl)n1] | 4-{[3-(Aminomethyl)-1-(2-chlorophenyl)-1H-pyrazol-5- yl]methyl}phenol |
| 40 | 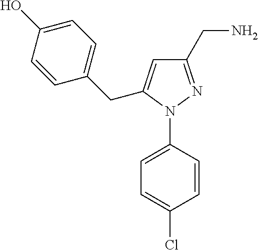 [SMILES: NCc1cc(Cc2ccc(O)cc2)n(-c2ccc(Cl)cc2)n1] | 4-{[3-(Aminomethyl)-1-(4-chlorophenyl)-1H-pyrazol-5- yl]methyl}phenol |
| 41 | 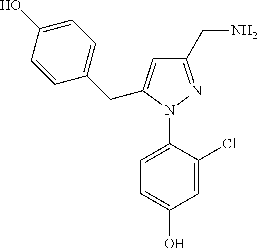 [SMILES: NCc1cc(Cc2ccc(O)cc2)n(-c2ccc(O)cc2Cl)n1] | 4-[3-(Aminomethyl)-5-(4-hydroxybenzyl)-1H-pyrazol- 1-yl]-3-chlorophenol |
| 42 | 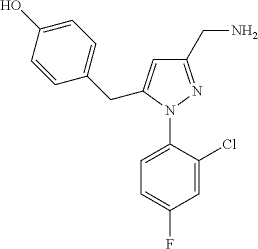 [SMILES: NCc1cc(Cc2ccc(O)cc2)n(-c2ccc(F)cc2Cl)n1] | 4-{[3-(Aminomethyl)-1-(2-chloro-4-fluorophenyl)-1H- pyrazol-5-yl]methyl}phenol |
| 43 | 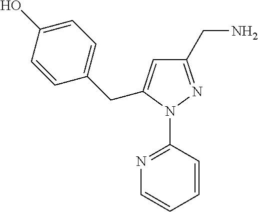 [SMILES: NCc1cc(Cc2ccc(O)cc2)n(-c2ccccn2)n1] | 4-{[3-(Aminomethyl)-1-(pyridin-2-yl)-1H-pyrazol-5- yl]methyl}phenol |
| 44 | 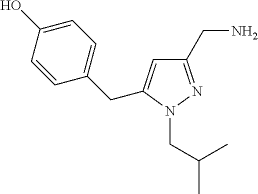 [SMILES: CC(C)Cn1nc(CN)cc1Cc1ccc(O)cc1] | 4-{[3-(Aminomethyl)-1-isobutyl-1H-pyrazol-5- yl]methyl}phenol |
| 45 | 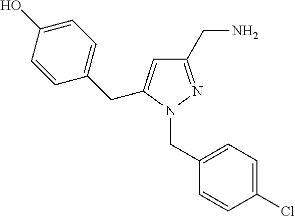 [SMILES: NCc1cc(Cc2ccc(O)cc2)n(Cc2ccc(Cl)cc2)n1] | 4-{[3-(Aminomethyl)-1-(4-chlorobenzyl)-1H-pyrazol-5- yl]methyl}phenol |
| 46 | 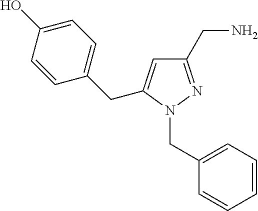 [SMILES: NCc1cc(Cc2ccc(O)cc2)n(Cc2ccccc2)n1] | 4-{[3-(Aminomethyl)-1-benzyl-1H-pyrazol-5- yl]methyl}phenol |
| 47 | 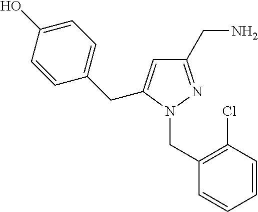 [SMILES: NCc1cc(Cc2ccc(O)cc2)n(Cc2ccccc2Cl)n1] | 4-{[3-(Aminomethyl)-1-(2-chlorobenzyl)-1H-pyrazol-5- yl]methyl}phenol |
| 48 | 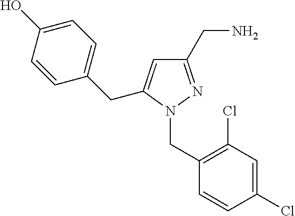 [SMILES: NCc1cc(Cc2ccc(O)cc2)n(Cc2ccc(Cl)cc2Cl)n1] | 4-{[3-(Aminomethyl)-1-(2-chlorobenzyl)-1H-pyrazol-5- yl]methyl}phenol |
| 49 | 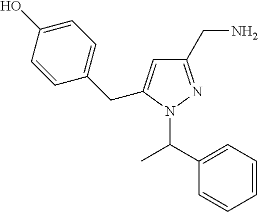 [SMILES: CC(c1ccccc1)n1nc(CN)cc1Cc1ccc(O)cc1] | 4-{[3-(Aminomethyl)-1-(1-phenylethyl)-1H-pyrazol-5- yl]methyl}phenol |
| 50 | 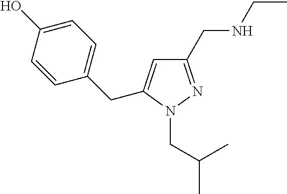 [SMILES: CCNCc1cc(Cc2ccc(O)cc2)n(CC(C)C)n1] | 4-({3-[(Ethylamino)methyl]-1-isobutyl-1H-pyrazol-5- yl}methyl)phenol |
| 51 | 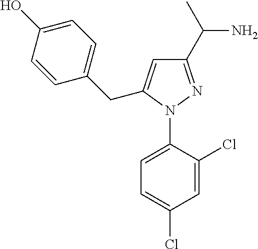 [SMILES: CC(N)c1cc(Cc2ccc(O)cc2)n(-c2ccc(Cl)cc2Cl)n1] | 4-{[3-(1-Aminoethyl)-1-(2,4-dichlorophenyl)-1H- pyrazol-5-yl]methyl}phenol |
| 52 | 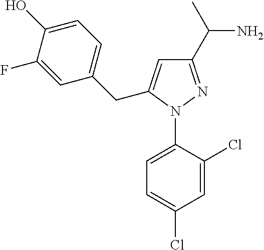 [SMILES: CC(N)c1cc(Cc2ccc(O)c(F)c2)n(-c2ccc(Cl)cc2Cl)n1] | 4-{[3-(1-Aminoethyl)-1-(2,4-dichlorophenyl)-1H- pyrazol-5-yl]methyl}-2-fluorophenol |
| 53 | 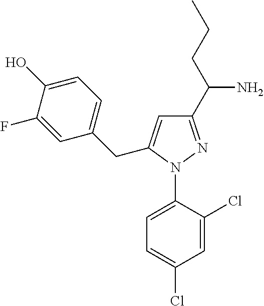 [SMILES: CCCC(N)c1cc(Cc2ccc(O)c(F)c2)n(-c2ccc(Cl)cc2Cl)n1] | 4-{[3-(1-Aminobutyl)-1-(2,4-dichlorophenyl)-1H- pyrazol-5-yl]methyl}-2-fluorophenol |
| 54 | 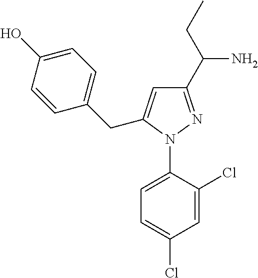 [SMILES: CCC(N)c1cc(Cc2ccc(O)cc2)n(-c2ccc(Cl)cc2Cl)n1] | 4-{[3-(1-Aminopropyl)-1-(2,4-dichlorophenyl)-1H- pyrazol-5-yl]methyl}phenol |
| 55 | 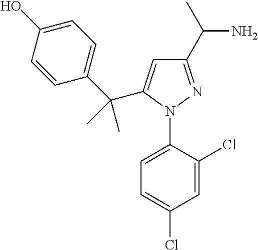 [SMILES: CC(N)c1cc(C(C)(C)c2ccc(O)cc2)n(-c2ccc(Cl)cc2Cl)n1] | 4-{2-[3-(1-Aminoethyl)-1-(2,4-dichlorophenyl)-1H- pyrazol-5-yl]propan-2-yl}phenol |
| 56 | 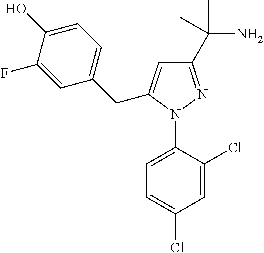 [SMILES: CC(C)(N)c1cc(Cc2ccc(O)c(F)c2)n(-c2ccc(Cl)cc2Cl)n1] | 4-{[3-(2-Aminopropan-2-yl)-1-(2,4-dichlorophenyl)- 1H-pyrazol-5-yl]methyl}-2-fluorophenol |
| 57 | 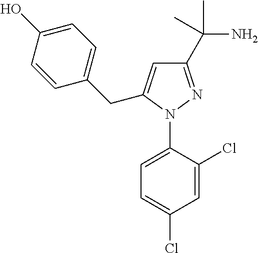 [SMILES: CC(C)(N)c1cc(Cc2ccc(O)cc2)n(-c2ccc(Cl)cc2Cl)n1] | 4-{[3-(2-Aminopropan-2-yl)-1-(2,4-dichlorophenyl)- 1H-pyrazol-5-yl]methyl}phenol |
| 58 | 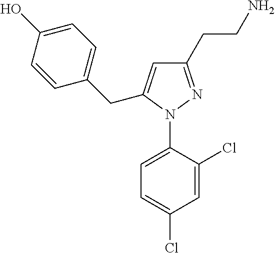 [SMILES: NCCc1cc(Cc2ccc(O)cc2)n(-c2ccc(Cl)cc2Cl)n1] | 4-{[3-(2-Aminoethyl)-1-(2,4-dichlorophenyl)-1H- pyrazol-5-yl]methyl}phenol |
| 59 | 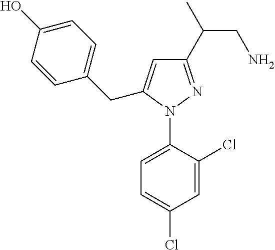 [SMILES: CC(CN)c1cc(Cc2ccc(O)cc2)n(-c2ccc(Cl)cc2Cl)n1] | 4-{[3-(1-Aminopropan-2-yl)-1-(2,4-dichlorophenyl)- 1H-pyrazol-5-yl]methyl}phenol |
| 60 | 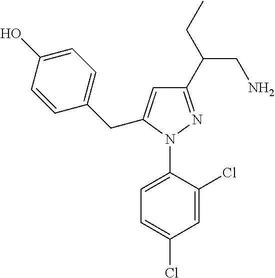 [SMILES: CCC(CN)c1cc(Cc2ccc(O)cc2)n(-c2ccc(Cl)cc2Cl)n1] | 4-{[3-(1-Aminobutan-2-yl)-1-(2,4-dichlorophenyl)-1H- pyrazol-5-yl]methyl}phenol |
| 61 | 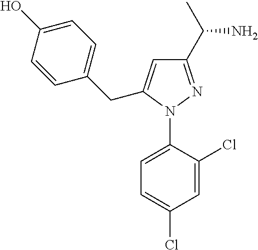 [SMILES: C[C@H](N)c1cc(Cc2ccc(O)cc2)n(-c2ccc(Cl)cc2Cl)n1] | (S)-4-{[3-(1-Aminoethyl)-1-(2,4-dichlorophenyl)-1H- pyrazol-5-yl]methyl}phenol |
| 62 | 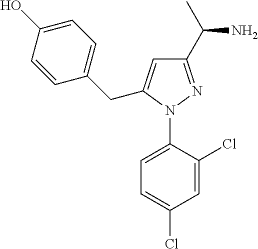 [SMILES: C[C@@H](N)c1cc(Cc2ccc(O)cc2)n(-c2ccc(Cl)cc2Cl)n1] | (R)-4-{[3-(1-Aminoethyl)-1-(2,4-dichlorophenyl)-1H- pyrazol-5-yl]methyl}phenol |
| 63 | 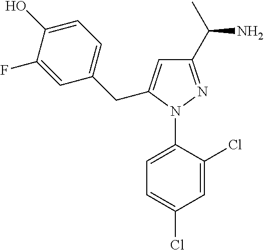 [SMILES: C[C@@H](N)c1cc(Cc2ccc(O)c(F)c2)n(-c2ccc(Cl)cc2Cl)n1] | (S)-4-{[3-(1-Aminoethyl)-1-(2,4-dichlorophenyl)-1H- pyrazol-5-yl]methyl}-2-fluorophenol |
| 64 | 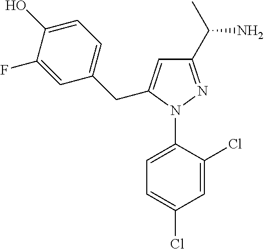 [SMILES: C[C@H](N)c1cc(Cc2ccc(O)c(F)c2)n(-c2ccc(Cl)cc2Cl)n1] | (R)-4-{[3-(1-Aminoethyl)-1-(2,4-dichlorophenyl)-1H- pyrazol-5-yl]methyl}-2-fluorophenol |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | Structure | Chemical name |
|---|---|---|
| 65 | 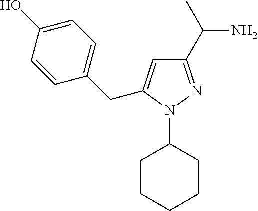 [SMILES: CC(N)c1cc(Cc2ccc(O)cc2)n(C2CCCCC2)n1] | 4-{[3-(1-Aminoethyl)-1-cyclohexyl-1H- pyrazol-5-yl]methyl}phenol |
| 66 | 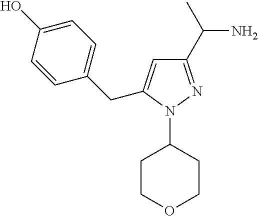 [SMILES: CC(N)c1cc(Cc2ccc(O)cc2)n(C2CCOCC2)n1] | 4-{[3-(1-Aminoethyl)-1-(tetrahydro-2H-pyran- 4-yl)-1H-pyrazol-5-yl]methyl}phenol |
| 67 | 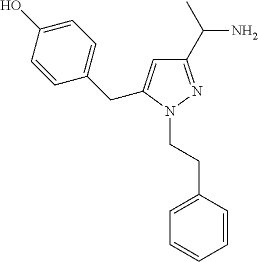 [SMILES: CC(N)c1cc(Cc2ccc(O)cc2)n(CCc2ccccc2)n1] | 4-{[3-(1-Aminoethyl)-1-phenethyl-1H-pyrazol- 5-yl]methyl}phenol |
| 68 | 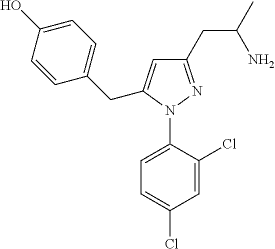 [SMILES: CC(N)Cc1cc(Cc2ccc(O)cc2)n(-c2ccc(Cl)cc2Cl)n1] | 4-{[3-(2-Aminopropyl)-1-(2,4-dichlorophenyl)- 1H-pyrazol-5-yl]methyl}phenol |
| 69 | 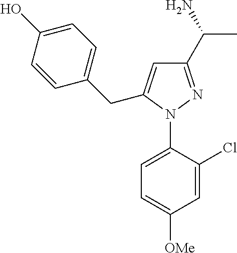 [SMILES: COc1ccc(-n2nc([C@@H](C)N)cc2Cc2ccc(O)cc2)c(Cl)c1] | (R)-4-{[3-(1-Aminoethyl)-1-(2-chloro-4- methoxyphenyl)-1H-pyrazol-5- yl]methyl}phenol |
| 70 | 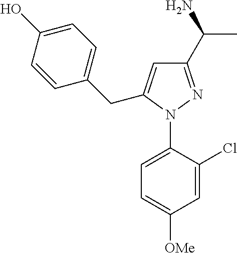 [SMILES: COc1ccc(-n2nc([C@H](C)N)cc2Cc2ccc(O)cc2)c(Cl)c1] | (S)-4-{[3-(1-Aminoethyl)-1-(2-chloro-4- methoxyphenyl)-1H-pyrazol-5- yl]methyl}phenol |
| 71 | 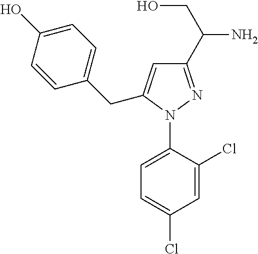 [SMILES: NC(CO)c1cc(Cc2ccc(O)cc2)n(-c2ccc(Cl)cc2Cl)n1] | 4-{[3-(1-Amino-2-hydroxyethyl)-1-(2,4- dichlorophenyl)-1H-pyrazol-5- yl]methyl}phenol |
| 72 | 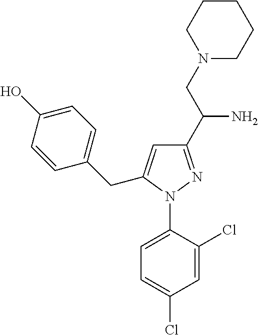 [SMILES: NC(CN1CCCCC1)c1cc(Cc2ccc(O)cc2)n(-c2ccc(Cl)cc2Cl)n1] | 4-({3-[1-Amino-2-(piperidin-1-yl)ethyl]-1-(2,4- dichlorophenyl)-1H-pyrazol-5- yl}methyl)phenol |
| 73 | 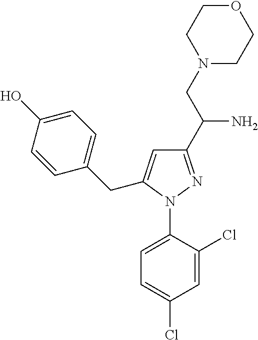 [SMILES: NC(CN1CCOCC1)c1cc(Cc2ccc(O)cc2)n(-c2ccc(Cl)cc2Cl)n1] | 4-{[3-(1-Amino-2-morpholinoethyl)-1-(2,4- dichlorophenyl)-1H-pyrazol-5- yl]methyl}phenol |
| 74 | 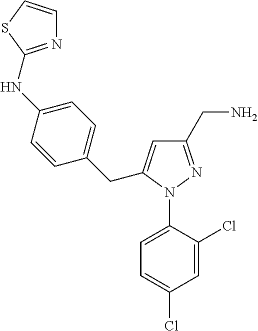 [SMILES: NCc1cc(Cc2ccc(Nc3nccs3)cc2)n(-c2ccc(Cl)cc2Cl)n1] | N-(4-{[3-(Aminomethyl)-1-(2,4- dichlorophenyl)-1H-pyrazol-5- yl]methyl}phenyl)thiazol-2-amine |
| 75 | 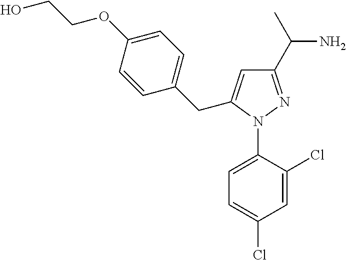 [SMILES: CC(N)c1cc(Cc2ccc(OCCO)cc2)n(-c2ccc(Cl)cc2Cl)n1] | 2-(4-{[3-(1-Aminoethyl)-1-(2,4- dichlorophenyl)-1H-pyrazol-5- yl]methyl}phenoxy)ethan-1-ol |
| 76 | 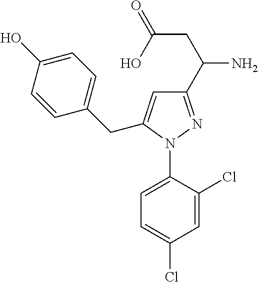 [SMILES: NC(CC(=O)O)c1cc(Cc2ccc(O)cc2)n(-c2ccc(Cl)cc2Cl)n1] | 3-Amino-3-[1-(2,4-dichlorophenyl)-5-(4- hydroxybenzyl)-1H-pyrazol-3-yl]propanoic acid |
| 77 | 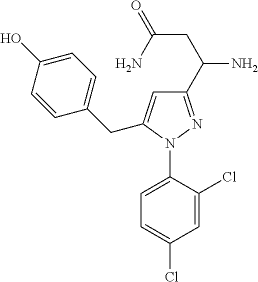 [SMILES: NC(=O)CC(N)c1cc(Cc2ccc(O)cc2)n(-c2ccc(Cl)cc2Cl)n1] | 3-Amino-3-[1-(2,4-dichlorophenyl)-5-(4- hydroxybenzyl)-1H-pyrazol-3-yl]propanamide |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compounds according to the invention of general Formula (I), having dual pharmacological activity towards both the α₂δ subunit of the voltage-gated calcium channel, and the μ-opioid receptor are selected from

examples 1, 4, 7, 17, 24, 27, 34, 37, 38, 41, 42, 51, 52, 53, 54, 56, 57, 58, 59, 60, 61 and 62; more preferably selected from examples 1, 7, 27, 37, 38, 41, 51, 52, 53, 56, 57, 58, 59, 60, 61 and 62; even more preferably selected from examples 1, 7, 27, 37, 38, 41, 51, 52, 56, 57, 58, 59, 60, 61 and 62,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;
wherein the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};
wherein the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆; wherein R₆, R_{6′} and R_{6″} are independently selected from hydrogen, unsubstituted C₁₋₆ alkyl, unsubstituted C₂₋₆ alkenyl and unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted aryl and substituted or unsubstituted heterocyclyl;
wherein the aryl or heterocyclyl in R₁, if substituted, is substituted with one or more substituent/s selected from halogen or —OR₆;
wherein R₆ is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted cycloalkyl and substituted or unsubstituted heterocyclyl;
wherein the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};
wherein the cycloalkyl, aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6″} are independently selected from hydrogen, unsubstituted C₁₋₆ alkyl, unsubstituted C₂₋₆ alkenyl and unsubstituted C₂₋₆ alkynyl;
and R_{6′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from unsubstituted C₁₋₆ alkyl, substituted or unsubstituted aryl, substituted or unsubstituted cycloalkyl and substituted or unsubstituted heterocyclyl;
wherein the cycloalkyl, aryl or heterocyclyl in R₁, if substituted, is substituted with one or more substituent/s selected from halogen or —OR₆;
wherein R₆ is selected from hydrogen and unsubstituted C₁₋₆ alkyl,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is substituted or unsubstituted aryl;
wherein said aryl or heterocyclyl in R₂, if substituted, is substituted with one or more substituent/s selected from halogen, —OR₇ or —NR₇S(O)₂R_{7′};
wherein R₇ and R_{7′} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7″} are independently selected from hydrogen, unsubstituted C₁₋₆ alkyl, unsubstituted C₂₋₆ alkenyl and unsubstituted C₂₋₆ alkynyl;
and wherein R_{7′″} is selected from hydrogen, unsubstituted C₁₋₆ alkyl, unsubstituted C₂₋₆ alkenyl, unsubstituted C₂₋₆ alkynyl, unsubstituted heterocyclyl, and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is substituted or unsubstituted aryl;
wherein said aryl or heterocyclyl in R₂, if substituted, is substituted with one or more substituent/s selected from halogen, —OR₇, —NR₇R_{7′″} and —NR₇S(O)₂R_{7′};
wherein R₇ and R_{7′} are independently selected from hydrogen and unsubstituted C₁₋₆ alkyl;
and wherein R_{7′″} is unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I), R₃ and R_{3′} are independently selected from hydrogen, substituted or unsubstituted C₁₋₆ alkyl, substituted or unsubstituted C₂₋₆ alkenyl, substituted or unsubstituted C₂₋₆ alkynyl,
wherein the alkyl, alkenyl or alkynyl in R₃ or R_{3′}, if substituted, is substituted with one or more substituent/s selected from —OR₈, —C(O)R₈, halogen, —CN, haloalkyl, haloalkoxy and —NR₈R_{8′″};
wherein R₈ is selected from hydrogen, unsubstituted C₁₋₈ alkyl, unsubstituted C₂₋₈ alkenyl and unsubstituted C₂₋₈ alkynyl;
and wherein R_{8′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₃ and R_{3′} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I), the alkyl, alkenyl or alkynyl, other than those defined in R₁, R₃ or R_{3′} if substituted, is substituted with one or more substituent/s selected from —OR₉, halogen, —CN, haloalkyl, haloalkoxy and —NR₉R_{9′″};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I), wherein, the alkyl, alkenyl or alkynyl, other than those defined in R₁, R₃ or R_{3′}, if substituted, is substituted with one or more substituent/s selected from —OR₉, halogen, —CN, haloalkyl, haloalkoxy, unsubstituted heterocyclyl, —C(O)OR₉, —C(O)NR₉R_{9′″} and —NR₉R_{9′″};
wherein R₉ is selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

wherein, the alkyl other than those defined in R₁, R₃ or R_{3′}, if substituted, is substituted with one or more substituent/s selected from —OR₉, unsubstituted heterocyclyl and —C(O)OR₉, —C(O)NR₉R_{9′″};
wherein R₉ hydrogen;
and R_{9′″} is hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I), the aryl, heterocyclyl or cycloalkyl other than those defined in R₁ or R₂, if substituted, is substituted with one or more substituent/s selected from halogen, —R₁₀, —OR₁₀, —NO₂, —NR₁₀R_{10′″}, NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —NR₁₀S(O)₂NR_{10′}R_{0″} and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{10′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the alkyl, alkenyl or alkynyl in R₁, if substituted, is substituted with one or more substituent/s selected from —OR₆, —C(O)R₆, halogen, —CN, haloalkyl, haloalkoxy and —NR₆R_{6′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the aryl or heterocyclyl in R₁, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,

the aryl or heterocyclyl in R₂, if substituted, is substituted with one or more substituent/s selected from halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₃ of any of the embodiments of the present invention,

the alkyl, alkenyl or alkynyl in R₃, if substituted, is substituted with one or more substituent/s selected from —OR₈, —C(O)R₈, halogen, —CN, haloalkyl, haloalkoxy and —NR₈R_{8′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R_{3′} of any of the embodiments of the present invention,

the alkyl, alkenyl or alkynyl in R_{3′}, if substituted, is substituted with one or more substituent/s selected from —OR₈, —C(O)R₈, halogen, —CN, haloalkyl, haloalkoxy and —NR₈R_{8′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl, other than those defined in R₁, R₃ or R_{3′}, if substituted, is substituted with one or more substituent/s selected from —OR₉, halogen, —CN, haloalkyl, haloalkoxy and —NR₉R_{9′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to the cycloalkyl, aryl or heterocyclyl other than those defined in R₁, R₂ or R₆ of any of the embodiments of the present invention,

the aryl, heterocyclyl or cycloalkyl other than those defined in R₁ or R₂, if substituted, is substituted with one or more substituent/s selected from halogen, —R₁₀, —OR₁₀, —NO₂, —NR₁₀R_{10″}, NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —NR₁₀S(O)₂NR_{10′}R_{10″} and C(CH₃)₂OR₁₀;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment,
the cycloalkyl, aryl or heterocyclyl in R₁, if substituted, is substituted with one or more substituent/s selected from chlorine, fluorine, —OH, —OCH₃ or —OCH₂CH₃.

In a preferred embodiment,
the aryl or heterocyclyl in R₂, if substituted, is substituted with one or more substituent/s selected from fluorine, —OH, —OCH₃, —OCH₂CH₃, —OCH₂CH₂OH, —NHSO₂CH₃ or —NH-thiazole.

In a preferred embodiment,
the alkyl, alkenyl or alkynyl, other than those defined in R₁, R₃ or R_{3′}, if substituted, is substituted with one or more substituent/s selected —OH, —C(O)OH, —C(O)—NH₂, piperidine or morpholine.

In an embodiment of the compound according to the invention of general Formula (I),

the halogen is fluorine, chlorine, iodine or bromine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of general Formula (I)

the halogen is fluorine or chlorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the α2δ subunit, particularly the α2δ-1 subunit, of the voltage-gated calcium channel and the μ-opioid receptor it is a very preferred embodiment in which the compounds are selected which act as dual ligands of the α2δ subunit, particularly the α2δ-1 subunit, of the voltage-gated calcium channel and the μ-opioid receptor and especially compounds which have a binding expressed as Kᵢ responding to the following scales:

Kᵢ(μ) is preferably <1000 nM, more preferably <500 nM, even more preferably <100 nM.

Ki(α2δ1) is preferably <10000 nM, more preferably <5000 nM, even more preferably <500 nM or even more preferably <100 nM.

In the following the phrase “compound of the invention” is used. This is to be understood as any compound according to the invention as described above according to general Formula (I), (I′), (I^{2′}), (I^{3′}), (I^{4′}), (I′), (I^{6′}), (I^{7′}), (I^{8′}), (I^{9′}), (I^{9a′}) or (I^{10′}).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

In general the processes are described below in the experimental part. The starting materials are commercially available or can be prepared by conventional methods.

A preferred aspect of the invention is also a process for the production of a compound according to Formula (I), following schemes 1 or 2.

A preferred embodiment of the invention is a process for the production of a compound according to Formula (I), wherein, if not defined otherwise, m, n, R_{c}, R₁, R₂, R₃, R_{3′}, R₄, R_{4′}, R₅, R_{5′} and X have the meanings defined in the description.

For the sake of clarity the expression “a compound according to Formula (I), wherein R₁, etc. are as defined in the description” would (just like the expression “a compound of Formula (I) as defined in any one of e.g. claims 1 to 10” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (I) as defined in any one of claims 1 to 10”.

In a particular embodiment there is a process for the production of a compound according to Formula (I), wherein X is —C(RₓR_{x′})— or —O—, R₅ and R_{5′} are both hydrogen and n is 1,

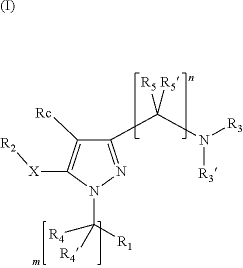

said process comprises the reductive amination of compounds of formula XIVex,

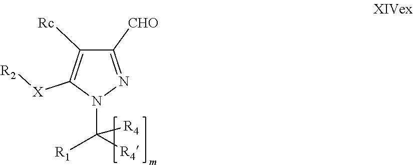

with an amine of formula XV

HNR₃R₃′ XV.

In a particular embodiment there is a process for the production of a compound according to Formula (I), wherein X is —C(RₓR_{x′})— or —O—, R_{5′} is hydrogen and n is 1,

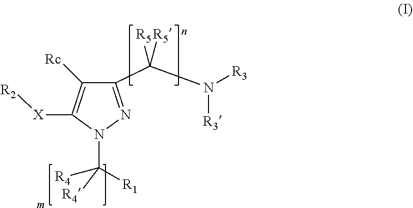

said process comprises the addition of an organometallic reagent of formula XVIII

R₅MgBr XVIII

to compounds of formula XVIIex

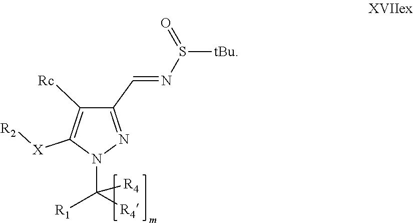

In a particular embodiment there is a process for the production of a compound according to Formula (I), wherein X is —C(RₓR_{x′})— or —O— and n is 1,

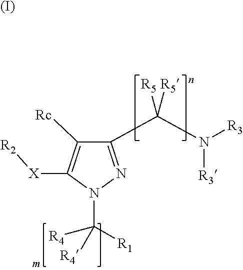

said process comprises the alkylation reaction of a compound of formula XXex

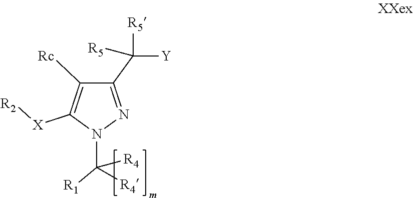

with an amine of formula XV

HNR₃R₃′ XV.

In a particular embodiment there is a process for the production of a compound according to Formula (I), wherein the compound of Formula (I) is a compound of Formula (Idₑₓ) and wherein X is —C(RₓR_{x′})— or —O—,

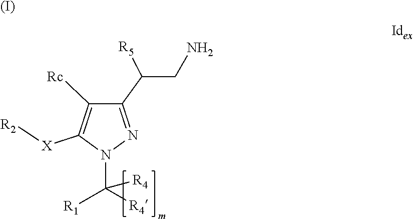

said process comprises the reaction of compounds of formula XXIex,

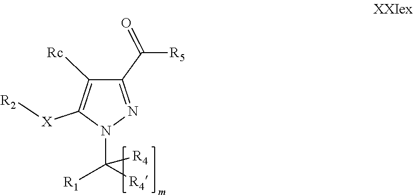

with tosylmethylisocyanide.

In a particular embodiment there is a process for the production of a compound according to Formula (I), wherein the compound of Formula (I) is a compound of Formula (Ieₑₓ) and wherein X is —C(RₓR_{x′})— or —O—,

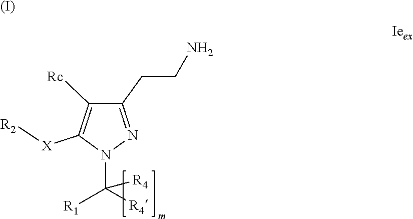

said process comprises the reduction of compounds of formula XXIIex

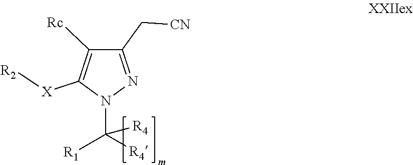

with a suitable reducing agent.

In a particular embodiment there is the use of a compound of Formula (Iₑₓ),

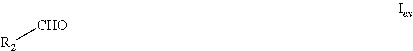

for the preparation of compounds of Formula (I).

In a particular embodiment there is the use of a compound of Formula (IIa) or (IIb),

Rc=H IIa

R_{c}=Alkyl IIb

for the preparation of compounds of Formula (I).

In a particular embodiment there is the use of a compound of Formula (III),

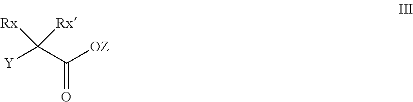

for the preparation of compounds of Formula (I).

In a particular embodiment there is the use of a compound of Formula (IV),

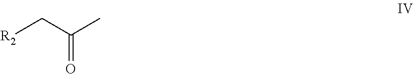

for the preparation of compounds of Formula (I).

In a particular embodiment there is the use of a compound of Formula (V),

RₓY V

for the preparation of compounds of Formula (I).

In a particular embodiment there is the use of a compound of Formula (VI),

RcY VI

for the preparation of compounds of Formula (I).

In a particular embodiment there is the use of a compound of Formula (VII),

(COOZ)₂ VII

for the preparation of compounds of Formula (I).

In a particular embodiment there is the use of a compound of Formula (VIII),

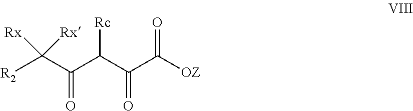

for the preparation of compounds of Formula (I).

In a particular embodiment there is the use of a compound of Formula (IX),

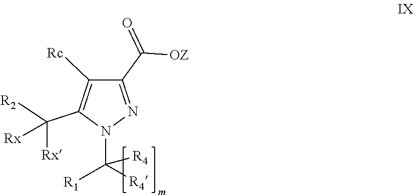

for the preparation of compounds of Formula (I).

In a particular embodiment there is the use of a compound of Formula (X),

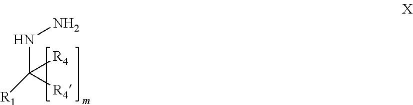

for the preparation of compounds of Formula (I).

In a particular embodiment there is the use of a compound of Formula (XI),

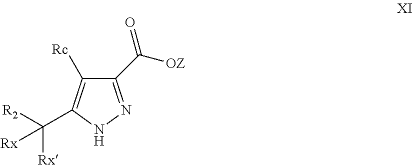

for the preparation of compounds of Formula (I).

In a particular embodiment there is the use of a compound of Formula (XII),

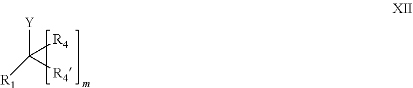

for the preparation of compounds of Formula (I).

In a particular embodiment there is the use of a compound of Formula (XIII),

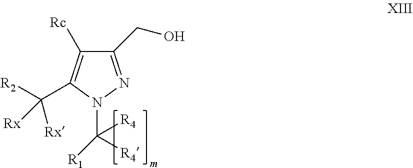

for the preparation of compounds of Formula (I).

In a particular embodiment there is the use of a compound of Formula (XIV),

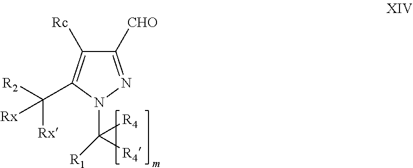

for the preparation of compounds of Formula (I).

In a particular embodiment there is the use of a compound of Formula (XIVex),

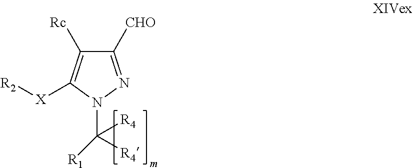

for the preparation of compounds of Formula (I).

In a particular embodiment there is the use of a compound of Formula (XV),

HNR₃R₃′ XV

for the preparation of compounds of Formula (I).

In a particular embodiment there is the use of a compound of Formula (XVI),

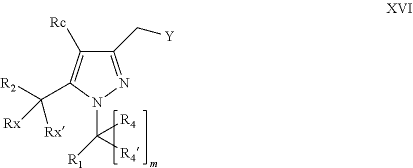

for the preparation of compounds of Formula (I).

In a particular embodiment there is the use of a compound of Formula (XVIex),

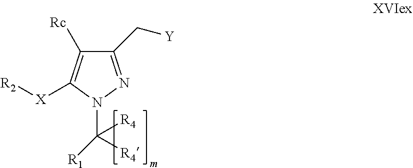

for the preparation of compounds of Formula (I).

In a particular embodiment there is the use of a compound of Formula (XVII),

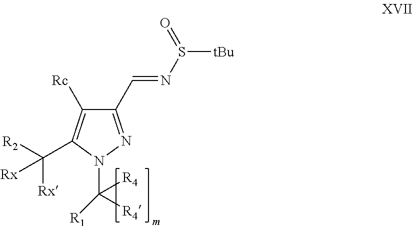

for the preparation of compounds of Formula (I).

In a particular embodiment there is the use of a compound of Formula (XVIIex),

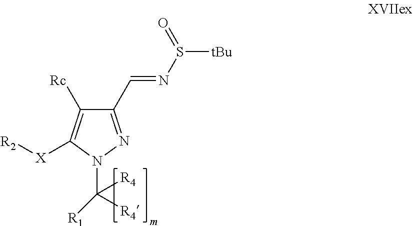

for the preparation of compounds of Formula (I).

In a particular embodiment there is the use of a compound of Formula (XVIII),

R₅MgBr XVIII

for the preparation of compounds of Formula (I).

In a particular embodiment there is the use of a compound of Formula (XIX),

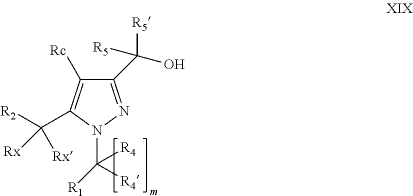

for the preparation of compounds of Formula (I).

In a particular embodiment there is the use of a compound of Formula (XX),

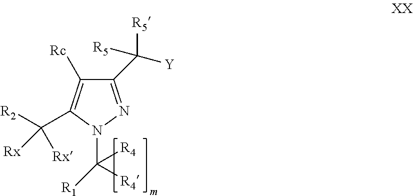

for the preparation of compounds of Formula (I).

In a particular embodiment there is the use of a compound of Formula (XXex),

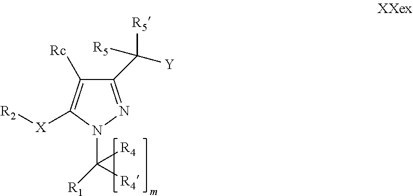

for the preparation of compounds of Formula (I).

In a particular embodiment there is the use of a compound of Formula (XXI),

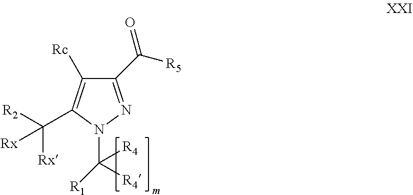

for the preparation of compounds of Formula (I).

In a particular embodiment there is the use of a compound of Formula (XXIex),

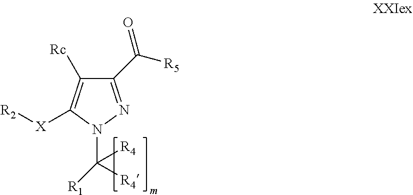

for the preparation of compounds of Formula (I).

In a particular embodiment there is the use of a compound of Formula (XXII),

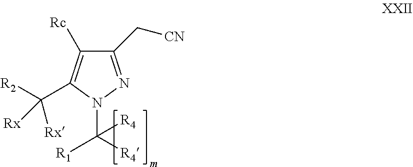

for the preparation of compounds of Formula (I).

In a particular embodiment there is the use of a compound of Formula (XXIIex),

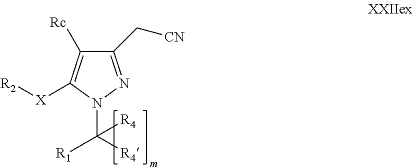

for the preparation of compounds of Formula (I).

In a particular embodiment there is the use of compounds of Formula Iₑₓ, IIa, IIb, III, IV, V, VI, VII, VIII, IX, X, XI, XII, XIII, XIV, XIVₑₓ, XV, XVI, XVIₑₓ, XVII, XVIIₑₓ, XVIII, XIX, XX, XXₑₓ, XXI, XXIₑₓ or XXII, XXIIₑₓ,

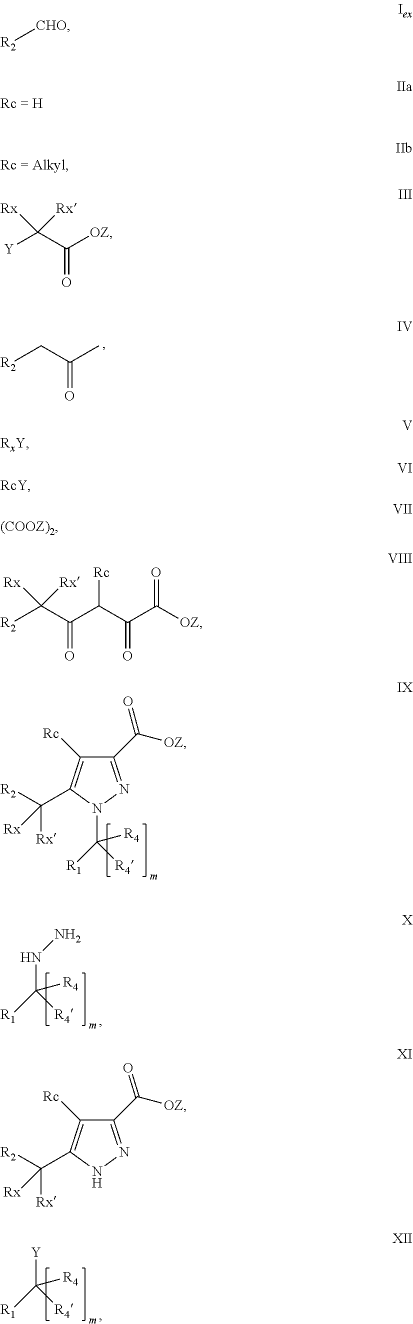

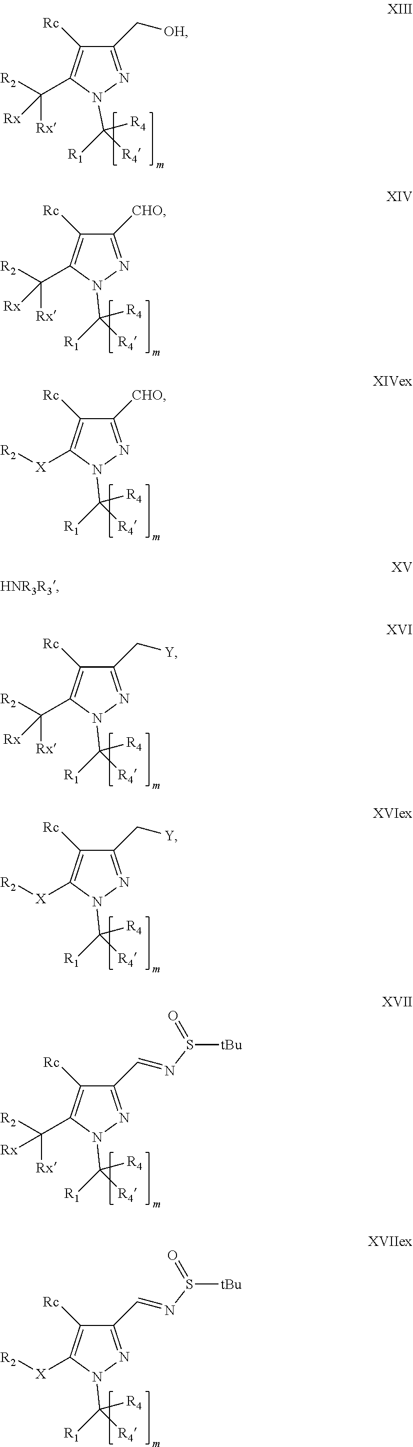

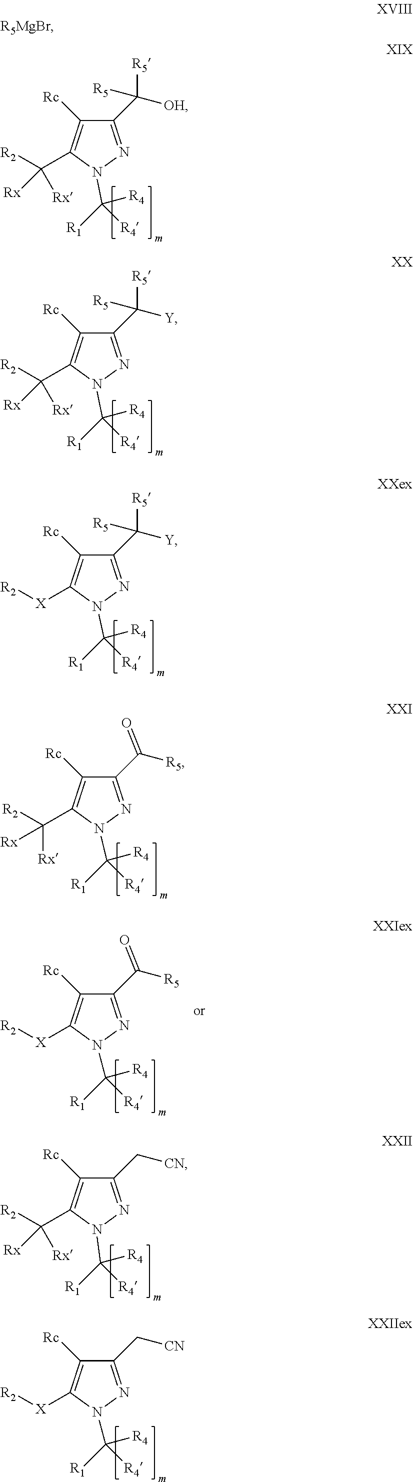

for the preparation of a compound of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

Biological Activity

Pharmacological Study

Human α₂δ-1 Subunit of Caᵥ2.2 Calcium Channel Assay

Human α₂δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl₂ 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

General Experimental Part (Methods and Equipment of the Synthesis and Analysis

A process is described in Scheme 1 for the preparation of compounds of general formula I, wherein R₁ to R₅, R_{c}, m and n have the meanings defined above and X is CRₓR_{x′}.

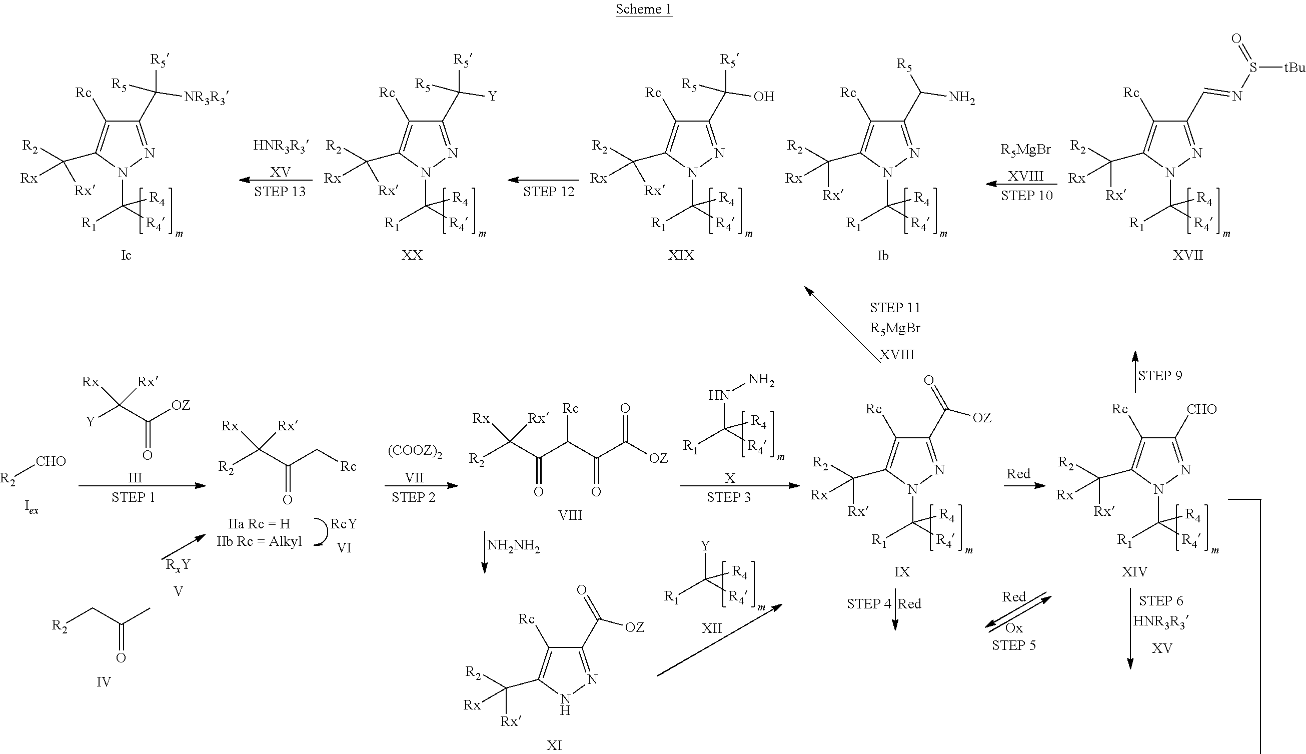

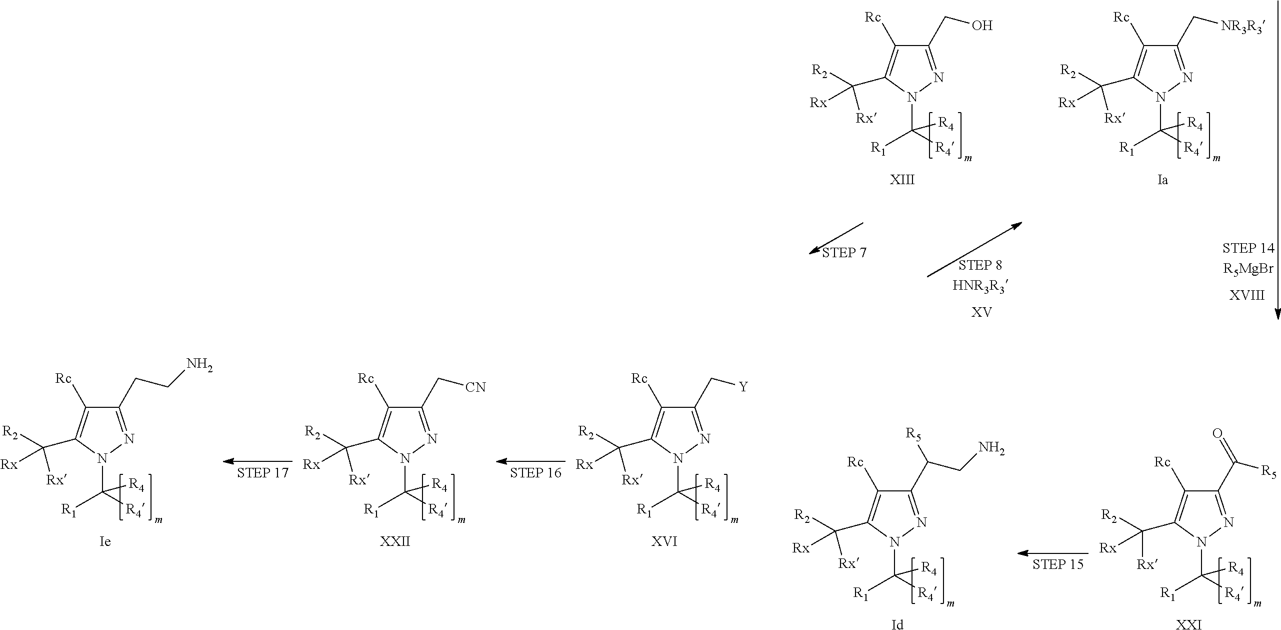

where, Z is an alkyl group and Y is a leaving group such as an halogen atom.

Step 1:

A compound of formula II can be prepared from the corresponding aldehyde of formula Iₑₓ via Darzens homologation, which involves treatment with an halogenated ester derivative of formula III in the presence of a base, such as potassium tert-butoxyde in a polar solvent, such as tetrahydrofuran followed by treatment with sodium hydroxide at a suitable temperature comprised between room temperature and the solvent reflux temperature, preferably heating, followed by decarboxylation in the presence of an inorganic acid, such as HCl, at a suitable temperature comprised between room temperature and the solvent reflux temperature, preferably heating.

Alternatively, compounds of formula II can be obtained by alkylation of a ketone of formula IV with an alkylating agent of formula V. The alkylation reaction is carried out in a suitable polar solvent, such as tetrahydrofuran in the presence of an inorganic base, such as K₂CO₃, or an organic base such as potassium tert-butoxide, at a suitable temperature comprised between room temperature and the solvent reflux temperature, preferably heating, or alternatively, the reaction can be carried out in a microwave reactor.

Alternatively, further alkylation with one equivalent of an alkylating agent of formula VI can produce the substituted derivatives of formula IIb.

Step 2:

A compound of formula VIII can be prepared by acylation of a compound of formula II with an oxalate of formula VII. The acylation reaction is carried out in a suitable polar solvent, such as tetrahydrofuran, in the presence of a base, such as NaH, at a suitable temperature comprised between room temperature and the solvent reflux temperature, preferably heating, or alternatively, the reaction can be carried out in a microwave reactor.

Step 3:

A compound of formula IX can be obtained by reaction of a compound of formula VIII and a hydrazine derivative of formula X. The reaction is carried out in a suitable polar solvent, such as ethanol, optionally in the presence of an acid, such as acetic acid, at a suitable temperature comprised between room temperature and the solvent reflux temperature, preferably heating, or alternatively, the reaction can be carried out in a microwave reactor.

Alternatively, compounds of formula IX can be prepared by reaction of a compound of formula VIII with hydrazine to give a compound of formula XI, following the conditions described above. The compound of formula XI is then substituted with a compound of formula XII, in the presence of a base, such as NaH or potassium tert-butoxide, in a suitable solvent, such as dimethylformamide or tetrahydrofuran, at a suitable temperature comprised between room temperature and the solvent reflux temperature, preferably heating, or alternatively, the reaction can be carried out in a microwave reactor.

Step 4:

A compound of formula XIII can be obtained by reduction of compounds of formula IX, using a suitable reducing agent, such as LiBH₄, in a suitable solvent, such as diethyl ether and at a suitable temperature, preferably room temperature.

Step 5:

A compound of formula XIV can be obtained by oxidation of compounds of formula XIII using a suitable oxidant, such as MnO₂, in a suitable solvent, such as dichloromethane at a suitable temperature, such as room temperature.

Alternatively compounds of formula XIV can be obtained directly from IX using a suitable reducing agent, such as DIBAL.

Step 6:

A compound of formula Ia can be obtained by reductive amination of compounds of formula XIV with an amine of formula XV, in the presence of a reductive reagent, preferably sodium triacetoxyborohydride, in a suitable solvent, preferably dichloromethane, at a suitable temperature comprised between room temperature and the solvent reflux temperature, preferably at room temperature.

Alternatively, if R₃ and R_{3′} are hydrogen, a compound of formula Ia can be obtained by reaction of compounds of formula XIV with NH₂OH.HCl in the presence of a base, such as triethylamine, in a suitable solvent, such as dichloromethane, at a suitable temperature comprised between room temperature and the solvent reflux temperature, preferably at room temperature, followed by reduction of the intermediate oxime, using a suitable reducing agent, such as Zn dust in a suitable solvent, such as acetic acid, at a suitable temperature comprised between room temperature and the solvent reflux temperature, preferably at room temperature.

Step 7:

Alternatively, compounds of formula Ia can be obtained from compounds of formula XIII via a two-step procedure that involves conversion of the hydroxyl function of XIII to a leaving group to afford compounds XVI, followed by alkylation with amines of formula XV. The formation of XVI can be carried out using mesyl chloride in the presence of a base, such as triethylamine, in a suitable solvent, such as dichloromethane, at a suitable temperature comprised between room temperature and the solvent reflux temperature, preferably at room temperature.

Step 8:

The alkylation reaction can be carried out in the presence of a base, such as triethylamine, in a suitable solvent, such as acetonitrile, at a suitable temperature comprised between room temperature and the solvent reflux temperature, preferably at room temperature.

Alternatively, if R₃ and R_{3′} are hydrogen, a compound of formula Ia can be obtained by reaction of compounds of formula XVI with sodium azide in a suitable solvent, such as dimethylformamide, at a suitable temperature comprised between room temperature and the solvent reflux temperature, preferably at room temperature, followed by reduction using a suitable reducing agent, such as SnCl₂, in a suitable solvent, such as ethanol, at a suitable temperature comprised between room temperature and the solvent reflux temperature, preferably at room temperature.

Step 9:

A compound of formula XVII can be obtained by condensation of compounds of formula XIV with a sulfinamide, preferably 2-methylpropane-2-sulfinamide, in the presence of a base, such as cesium carbonate, in a suitable solvent, such as dichloromethane, at a suitable temperature comprised between room temperature and the solvent reflux temperature, preferably at reflux.

Step 10:

A compound of formula Ib can be obtained by addition of an organometallic reagent of formula XVIII to compounds of formula XVII, in a suitable solvent, such as dichloromethane, at a suitable temperature comprised between room temperature and the solvent reflux temperature, preferably at room temperature, followed by treatment in acidic medium to cleave the sulfonamide moiety, using an inorganic acid, such as HCl in a suitable solvent, such as methanol, at a suitable temperature comprised between room temperature and the solvent reflux temperature, preferably at room temperature.

Step 11:

A compound of formula XIX can be obtained by addition of an organometallic reagent of formula XVIII to compounds of formula IX, in a suitable solvent, such as tetrahydrofuran, at a suitable temperature comprised between room temperature and the solvent reflux temperature, preferably at room temperature.

Step 12:

A compound of formula XX can be obtained from a compound of formula XIX by reaction with mesyl chloride in the presence of a base, such as triethylamine, in a suitable solvent, such as dichloromethane, at a suitable temperature comprised between room temperature and the solvent reflux temperature, preferably at room temperature.

Step 13:

A compound of formula Ic can be obtained by the alkylation reaction of a compound of formula XX with an amine of formula XV in the presence of a base, such as triethylamine, in a suitable solvent, such as acetonitrile, at a suitable temperature comprised between room temperature and the solvent reflux temperature, preferably at room temperature.

Alternatively, if R₃ and R_{3′} are hydrogen, a compound of formula Ic can be obtained by reaction of compounds of formula XX with sodium azide in a suitable solvent, such as dimethylformamide, at a suitable temperature comprised between room temperature and the solvent reflux temperature, preferably at room temperature, followed by reduction using a suitable reducing agent, such as SnCl₂ in a suitable solvent, such as ethanol, at a suitable temperature comprised between room temperature and the solvent reflux temperature, preferably at room temperature.

Step 14:

A compound of formula XXI can be obtained by addition of an organometallic reagent of formula XVIII to compounds of formula XIV, in a suitable solvent, such as tetrahydrofuran, at a suitable temperature comprised between 0° C. and the solvent reflux temperature, preferably at 0° C., followed by oxidation using a suitable oxidant, such as MnO₂, in a suitable solvent, such as dichloromethane at a suitable temperature, such as room temperature.

Step 15:

A compound of formula Id can be obtained by reaction of compounds of formula XXI, with tosylmehtylisocyanide in a suitable solvent, such as mixtures of dimethoxyethane and tert-butanol, in the presence of a base, preferably t-BuOK, at a suitable temperature comprised between room temperature and the solvent reflux temperature, preferably at room temperature.

Step 16:

A compound of formula XXII can be obtained from compounds of formula XVI by reaction with NaCN in a suitable solvent, such as dimethylformamide, at a suitable temperature comprised between room temperature and the solvent reflux temperature, preferably at room temperature.

Step 17:

A compound of formula Ie can be obtained by reduction of compounds of formula XXII with a suitable reducing agent, such as borane, in a suitable solvent, such as tetrahydrofuran, at a suitable temperature comprised between room temperature and the solvent reflux temperature, preferably at reflux temperature.

A process is described in Scheme 2 for the preparation of compounds of general formula I, wherein R_{c} and R₁ to R₅, m and n have the meanings defined above and X is an oxygen atom.

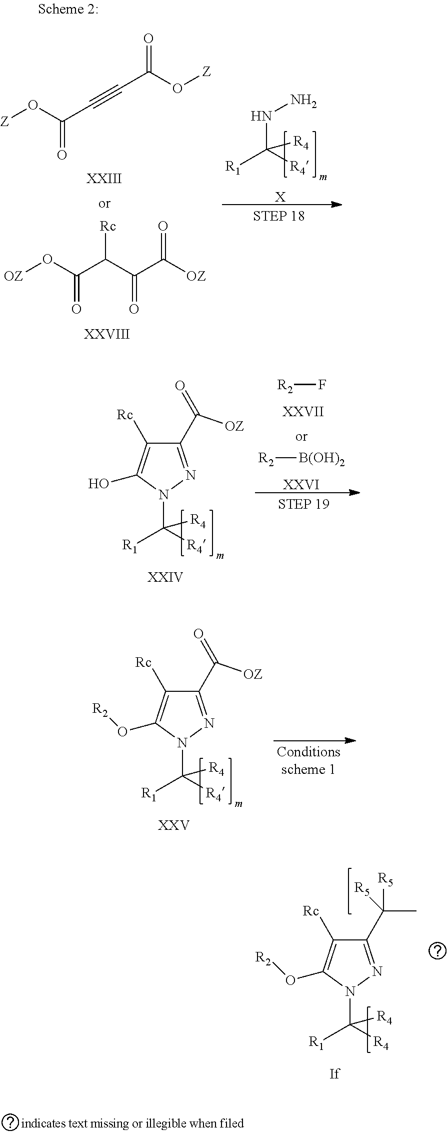

Step 18:

A compound of formula XXIV can be prepared by condensation of compounds of formula X, with dimethyl but-2-ynedioate (XXIII) in the presence of a base, such as K₂CO₃, in a suitable solvent, such as ethanol, at a suitable temperature comprised between room temperature and the solvent reflux temperature, preferably at reflux temperature.

Alternatively, compounds XXIV can be prepared from compounds of formula XXVIII using similar conditions

Step 19:

A compound of formula XXV can be prepared by copper-catalyzed coupling of compounds of formula XXIV with a boronic acid of formula XXVI, using a suitable copper catalyst, such as Cu(OAc)₂, a suitable base, such as pyridine, a suitable solvent such as dichloromethane, at a suitable temperature comprised between room temperature and the solvent reflux temperature, preferably at room temperature.

Alternatively, a compound of formula XXV can be obtained by reaction of a compound of formula XXIV with a fluoro derivative of formula XXVII in the presence of a base, such as K₂CO₃, in a suitable solvent, such as ethanol, at a suitable temperature comprised between room temperature and the solvent reflux temperature, preferably at reflux temperature.

Compounds of formula If can be obtained from compounds of formula XXV using analogous methods to those described above for the preparation of compounds Ia-e.

Additionally, different interconversion methods can be used to prepare the different intermediates of schemes 1 and 2 and compounds of general formula I:

By reaction of a compound containing an alkoxy group, to provide a hydroxyl derivative, by any suitable method, such as treatment with a Lewis acid, such as boron tribromide in a suitable solvent, such as dichloromethane, at a suitable temperature comprised between room temperature and the solvent reflux temperature, preferably at room temperature.

By reduction of a nitro derivative to an amino derivative by any suitable method, such as treatment with Fe, in a suitable solvent, such as water, at a suitable temperature comprised between room temperature and the solvent reflux temperature, preferably heating.

By deprotection reaction of a compound of formula I that contains an amine protecting group such as a carbamate, preferably tert-butoxy carbonyl, by any suitable method, such as treatment with an acid, preferably HCl or trifluoroacetic acid in an appropriate solvent such as 1,4-dioxane, DCM, ethyl acetate or a mixture of an organic solvent and water.

By reductive amination reaction of a compound of formula I that contains an amino group with an aldehyde, preferably carried out with a reductive reagent, preferably sodium triacetoxyborohydride, in an organic solvent, preferably DCE, in the presence of an organic base, preferably DIPEA or TEA. Alternatively, the reaction can be carried out in the presence of an acid, preferably acetic acid.

Compounds of formula I, III, IV, V, VI, VII, X, XII, XV, XVIII, XXVI and XXVII are commercially available or can be prepared from commercially available reagents using methods described in the literature.

### EXAMPLES

### Intermediates and Examples

The following abbreviations are used in the examples:

Ac: acetyl

br s: broad singlet

Bu: butyl

C: Celsius

DHP: 1,2-dihydropyran

DME: 1,2-dimethoxyethane

DMF: dimethylformamide

ESI: electrospray ionization

Et: ethyl

EtOH: ethanol

Et₂O: diethyl ether

EtOAc: ethyl acetate

Ex: example

g: gram

h: hour/s

HPLC: high-performance liquid chromatography

Hz: hertz

INT: intermediate

L: liter

m: meter, mili, multiplet

M: molar, molecular mass

m/z: mass-to-charge ratio

Me: methyl

MeOH: methanol

MS: mass spectrometry

min: minutes

NMR: nuclear magnetic resonance

pH: -logarithm of hydrogen ion concentration

PPTS: pyridinium p-toluenesulfonate

Py: pyridine

Ret: retention

rt: room temperature

TFA: trifluoroacetic acid

THF: tetrahydrofuran

THP: tetrahydropyranyl

TLC: thin layer chromatography

TOSMIC: p-toluenesulfonylmethyl isocyanide

w/w: weight/weight ratio

wt: weight

Xantphos: 4,5-bis(diphenylphosphino)-9,9-dimethylxanthene

The following methods were used to obtain the HPLC-MS data:

A: Column Kinetex C18 5 μm, 2.1×50 mm; flow rate: 0.30 mL/min; A: CH₃CN:MeOH (1:1); B: water; C: 100 mM ammonium acetate pH 7; gradient A:B:C: 3 min in 10:85:5+from 10:85:5 to 95:0:5 in 6 min+6 min in 95:0:5.

B: Column Luna C18 (2) 5 μm, 2.0×50 mm; flow rate: 0.30 mL/min; A: CH₃CN:MeOH (1:1); B: water; C: 100 mM ammonium acetate pH 7; gradient A:B:C: 3 min in 10:85:5+from 10:85:5 to 95:0:5 in 6 min+6 min in 95:0:5.

C: Column: SunFire C18, 5 μm, 2.1×50 mm; flow rate: 0.30 mL/min; A: CH₃CN:MeOH (1:1); B: water; C: 100 mM ammonium acetate pH 7; gradient: 2 min in 10:85:5+from 10:85:5 to 95:0:5 in 2 min+5 min in 95:0:5.

D: Column: SunFire C18, 5 μm, 2.1×50 mm; flow rate: 0.30 mL/min; A: CH₃CN:MeOH (1:1); B: water; C: 100 mM ammonium acetate pH 7; gradient: 3 min in 10:85:5+from 10:85:5 to 95:0:5 in 6 min+6 min in 95:0:5.

E: Column Kinetex C18 5 μm, 2.1×50 mm; flow rate: 0.30 mL/min; A: CH₃CN:MeOH (1:1); B: water; C: HCOOH 0.2%; gradient A:B:C: 4 min in 5:90:5+from 5:90:5 to 85:10:5 in 6 min+5 min in 85:10:5.

F: Column Kinetex C18 5 μm, 2.1×150 mm; flow rate: 0.35 mL/min; A: CH₃CN:MeOH (1:1); B: water; C: 100 mM ammonium acetate pH 7; gradient A:B:C: 5 min in 5:90:5+from 5:90:5 to 95:0:5 in 15 min+10 min in 95:0:5.

G: Column XTerra MS C18 3.5 μm, 2.1×100 mm; flow rate: 0.30 mL/min; A: CH₃CN:MeOH (1:1); B: water; C: 100 mM ammonium acetate buffer pH 9 (NH₄OH); gradient A:B:C: 3 min in 10:85:5+from 10:85:5 to 95:0:5 in 17 min+10 min in 95:0:5.

H: Column XTerra MS C18 3.5 μm, 2.1×100 mm; flow rate: 0.30 mL/min; A: CH₃CN:MeOH (1:1); B: water; C: 100 mM ammonium acetate buffer pH 9 (NH₄OH); gradient A:B:C: 5 min in 15:80:5+from 15:80:5 to 80:15:5 in 15 min+10 min in 80:15:5.

I: Column: SunFire C18, 3.5 μm, 2.1×100 mm; flow rate: 0.30 mL/min; A: CH₃CN:MeOH (1:1); B: water; C: 100 mM ammonium acetate pH 7; gradient A:B:C: 5 min in 10:85:5+from 10:85:5 to 95:0:5 in 15 min+10 min in 95:0:5.

### Intermediate A1. 2-(2-Chloro-4-methoxyphenyl)hydrazine hydrochloride

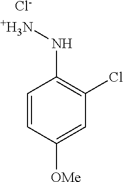 [SMILES: [Cl-] ; COc1ccc(N[NH3+])c(Cl)c1]

### a) 2-Chloro-4-methoxyaniline

Fe (5.10 g, 91.69 mmol) was added to a suspension of 2-chloro-4-methoxy-1-nitrobenzene (1.72 g, 9.17 mmol) and NH₄Cl (1.96 g, 36.68 mmol) in water (22 mL) and MeOH (70 mL). The reaction mixture was warmed up to 50° C. and stirred at this temperature for 1 h. The mixture was cooled down to rt, filtered and rinsed with water (2×15 mL). The filtrate was extracted with EtOAc (2×50 mL); the combined organic layers were dried over Na₂SO₄ (anhydrous), filtered and concentrated. The crude residue was purified by flash chromatography on SiO₂ (10→22% EtOAc/hexanes), to give 2-chloro-4-methoxyaniline (orange oil, 1.24 g, 86% yield).

HPLC-MS (Method A): Ret, 7.37 min; ESI⁺-MS m/z: 158 (M+1).

### B) Title Compound

A solution of NaNO₂ (0.80 g, 11.52 mmol) in water (3 mL) was added dropwise to a 0° C. cooled suspension of the previous compound (1.21 g, 7.68 mmol) in HCl (6 M aqueous solution, 9 mL). The reaction mixture was stirred for 1.5 h, SnCl₂ (6.06 g, 26.87 mmol) was added portionwise and stirring at 0° C. continued for 4 h. The reaction mixture was filtered and the solid was subsequently washed with cold water (2×4 mL), cold Et₂O (4 mL) and cold Et₂O/hexanes 1:1 (2×4 mL). The solid was dried under high vacuum to furnish (the title compound (cream solid, 1.78 g, quantitative yield).

HPLC-MS (Method A): Ret, 6.00 min; ESI⁺-MS m/z: 173 (M+1-HCl).

### Intermediate A2. 2-(2-Chloro-4-ethoxyphenyl)hydrazine hydrochloride

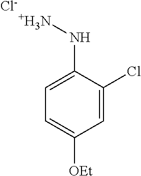 [SMILES: [Cl-] ; CCOc1ccc(N[NH3+])c(Cl)c1]

### a) 2-Chloro-4-ethoxyaniline

K₂CO₃ (3.89 g, 28.15 mmol) and ethyl iodide (0.99 mL, 12.40 mmol) were added to a suspension of 4-amino-3-chlorophenol hydrochloride (2.03 g, 11.28 mmol) in DMF (25 mL). The resulting suspension was stirred at rt for 20 h. After removal of volatiles by rotatory evaporation, the residue was diluted with EtOAc (80 mL) and was washed with water (60 mL). The aqueous phase was extracted with EtOAc (3×30 mL), the combined organic layers were dried over Na₂SO₄ (anhydrous), filtered and concentrated. The crude residue was purified by flash chromatography on SiO₂ (10→30% EtOAc/hexanes) to afford 2-chloro-4-ethoxyaniline (reddish oil, 0.77 g, 41% yield).

HPLC-MS (Method B): Ret, 9.39 min; ESI⁺-MS m/z: 172 (M+1).

### b) Title Compound

The title compound was obtained following the procedure described in Intermediate A1, step b) and using 2-chloro-4-ethoxyaniline as starting material.

HPLC-MS (Method B): Ret, 8.83 min; ESI⁺-MS m/z, 187 (M+1-HCl).

### Intermediate B1. 1-(3-Fluoro-4-methoxyphenyl)propan-2-one

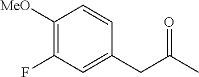

t-BuOK (4.37 g, 38.9 mmol) was added in portions to a −15° C. cooled solution of 3-fluoro-4-methoxybenzaldehyde (5.00 g, 32.4 mmol) and methyl 2-chloropropanoate (4.42 mL, 38.9 mmol) in THF (60 mL). After 30 min, NaOH (10% aqueous solution, 10 mL) was added and the mixture was warmed up to 40° C. and stirred at this temperature until full conversion was achieved (40 min). The reaction mixture was allowed to reach rt and volatiles were removed by rotatory evaporation until ¼ of the volume. The residue was diluted with 10 mL of water and was washed with Et₂O (2×30 mL), discarding the organic layers. The aqueous layer was placed in a round-bottom flask and toluene (30 mL) was added; the heterogeneous mixture was acidified with HCl (10% aqueous solution, 20 mL) and heated at 90° C. for 15 h. The reaction mixture was allowed to reach rt and was washed with NaOH (5% aqueous solution, 2×15 mL). The organic layer was dried over Na₂SO₄ (anhydrous), filtered and concentrated, affording the title compound (pale yellow oil, 3.70 g, 63% yield).

HPLC-MS (Method A): Ret, 7.92 min; ESI⁺-MS m/z: 183 (M+1).

### Intermediate B2. 1-(4-Ethoxyphenyl)propan-2-one

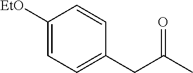

The title compound was obtained following the procedure described in Intermediate B1 and using 4-ethoxybenzaldehyde as starting material.

HPLC-MS (Method A): Ret, 8.62 min; ESI⁺-MS m/z, 179 (M+1).

### Intermediate B3. 3-(4-Methoxyphenyl)-3-methylbutan-2-one

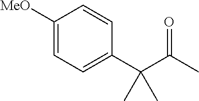

t-BuOK (6.15 g, 54.80 mmol) was added in portions to a −78° C. cooled solution of 1-(4-methoxyphenyl)propan-2-one (4.09 g, 24.91 mmol) and MeI (3.41 mL, 54.80 mmol) in THF (50 mL), forming a thick yellowish suspension. After 20 min the mixture was allowed to reach rt and stirred for 2 d. HCl (10% aqueous solution, 5 mL) was added and volatiles were removed by rotatory evaporation. The residue was diluted with EtOAc (100 mL) and was washed with brine (50 mL); the organic layer was dried over Na₂SO₄ (anhydrous), filtered and concentrated. The crude residue was purified by flash chromatography on SiO₂ (5% EtOAc/hexanes) to afford the title compound (yellow oil, 2.80 g, 58% yield).

HPLC-MS (Method B): Ret, 10.07 min; ESI⁺-MS m/z: 193 (M+1).

### Intermediate C1. Methyl 5-(4-methoxyphenyl)-2,4-dioxopentanoate

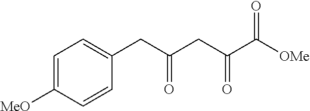 [SMILES: COC(=O)C(=O)CC(=O)Cc1ccc(OC)cc1]

NaH (60% mineral oil suspension, 1.75 g, 43.85 mmol) was added in small portions to a suspension of 1-(4-methoxyphenyl)propan-2-one (4.50 g, 27.41 mmol) in THF (40 mL). The reaction mixture was stirred at rt for 15 min and a solution of dimethyl oxalate (4.85 g, 41.11 mmol) in THF (20 mL) was added. The reaction mixture was warmed up to 70° C. and stirred at this temperature until full conversion was achieved (40 min). The reaction mixture was allowed to reach rt and poured into water (100 mL). HCl (10% aqueous solution) was added until pH=5-6 was reached and the aqueous layer was extracted with EtOAc (3×100 mL). The combined organic layers were dried over Na₂SO₄ (anhydrous), filtered and concentrated. The crude residue was purified by flash chromatography on SiO₂ (20→100% EtOAc/hexanes). The resulting solid was slurried with hexanes, filtered and dried to afford the title compound (pale yellow solid, 2.18 g, 32% yield).

HPLC-MS (Method A): Ret, 7.61 min; ESI⁺-MS m/z: 251 (M+1).

This method was used for the preparation of intermediates C₂-C₈ using suitable starting materials:

| | | | | Ret | MS |
|---|---|---|---|---|---|
| INT | Structure | Chemical name | Method | (min) | (M + H) |
| C2 | 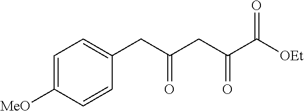 [SMILES: CCOC(=O)C(=O)CC(=O)Cc1ccc(OC)cc1] | Ethyl 5-(4- methoxyphenyl)- 2,4- dioxopentanoate | B | 9.48 | 265 |
| C3 | 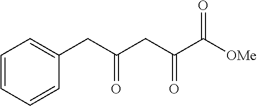 [SMILES: COC(=O)C(=O)CC(=O)Cc1ccccc1] | Methyl 2,4-dioxo- 5- phenylpentanoate | A | 6.87 | 221 |
| C4 | 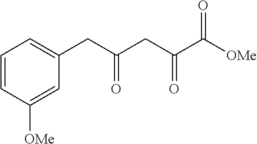 [SMILES: COC(=O)C(=O)CC(=O)Cc1cccc(OC)c1] | Methyl 5-(3- methoxyphenyl)- 2,4- dioxopentanoate | A | 7.43 | 251 |
| C5 | 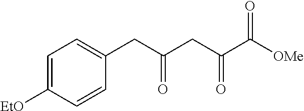 [SMILES: CCOc1ccc(CC(=O)CC(=O)C(=O)OC)cc1] | Methyl 5-(4- ethoxyphenyl)-2,4- dioxopentanoate | A | 8.19 | 265 |
| C6 | 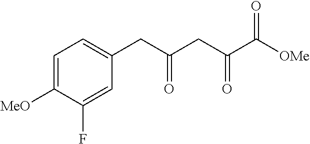 [SMILES: COC(=O)C(=O)CC(=O)Cc1ccc(OC)c(F)c1] | Methyl 5-(3-fluoro- 4-methoxyphenyl)- 2,4- dioxopentanoate | A | 7.04 | 269 |
| C7 | 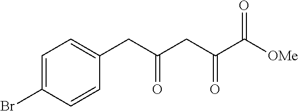 [SMILES: COC(=O)C(=O)CC(=O)Cc1ccc(Br)cc1] | Methyl 5-(4- bromophenyl)-2,4- dioxopentanoate | C | 6.41 | 299/301 |
| C8 | 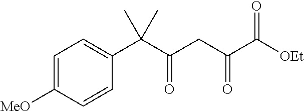 [SMILES: CCOC(=O)C(=O)CC(=O)C(C)(C)c1ccc(OC)cc1] | Ethyl 5-(4- methoxyphenyl)-5- methyl-2,4- dioxohexanoate | B | 10.80 | 293 |

### Intermediate C9. Ethyl 5-(4-hydroxyphenyl)-2,4-dioxopentanoate

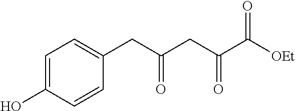 [SMILES: CCOC(=O)C(=O)CC(=O)Cc1ccc(O)cc1]

BBr₃ (1 M solution in CH₂Cl₂, 23.8 mL, 23.8 mmol) was added to a solution of ethyl 5-(4-methoxyphenyl)-2,4-dioxopentanoate (3.1 g, 11.9 mmol) in CH₂Cl₂ (35 mL) cooled at 0° C. and the mixture was stirred at rt for 6 h. The reaction mixture was poured into water (80 mL) and extracted with CH₂Cl₂ (2×90 mL). The combined organic layers were dried over Na₂SO₄ (anhydrous), filtered and concentrated to afford the title compound (brown oil, 1.2 g, 40% yield).

HPLC-MS (Method B): Ret, 8.07 min; ESI⁺-MS m/z: 251 (M+1).

### Intermediate C10. Methyl 5-(4-hydroxyphenyl)-2,4-dioxopentanoate

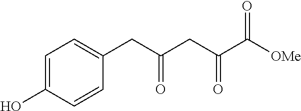 [SMILES: COC(=O)C(=O)CC(=O)Cc1ccc(O)cc1]

The title compound was obtained following the procedure described in Intermediate C9 and using methyl 5-(4-methoxyphenyl)-2,4-dioxopentanoate as starting material.

HPLC-MS (Method B): Ret, 6.31 min; ESI⁺-MS m/z, 237 (M+1).

### Intermediate D1. Methyl 1-(2,4-dichlorophenyl)-5-(4-methoxybenzyl)-1H-pyrazole-3-carboxylate

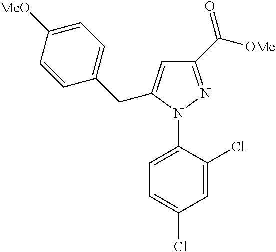 [SMILES: COC(=O)c1cc(Cc2ccc(OC)cc2)n(-c2ccc(Cl)cc2Cl)n1]

(2,4-Dichlorophenyl)hydrazine hydrochloride (2.08 g, 9.76 mmol) and AcOH (3.55 mL, 62.10 mmol) were added to a solution of methyl 5-(4-methoxyphenyl)-2,4-dioxopentanoate (2.22 g, 8.87 mmol) in EtOH (35 mL), the mixture was warmed up to 60° C. and stirred at this temperature for 3.5 h. The reaction mixture was allowed to cool down to rt and the solvent was concentrated off. The residue was poured into EtOAc (100 mL), washed with brine (100 mL) and with NaHCO₃ (saturated aqueous solution, 100 mL). The organic layer was dried over Na₂SO₄ (anhydrous), filtered and concentrated. The crude residue was purified by flash chromatography on SiO₂ (10→24% EtOAc/hexanes), to afford the title compound (orange oil, 2.84 g, 82% yield).

HPLC-MS (Method A): Ret, 10.52 min; ESI⁺-MS m/z: 391 (M+1).

This method was used for the preparation of intermediates D2-D20 using suitable starting materials:

| | | | | Ret | MS |
|---|---|---|---|---|---|
| INT | Structure | Chemical name | Method | (min) | (M + H) |
| D2 | 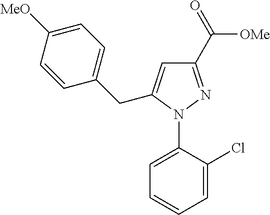 [SMILES: COC(=O)c1cc(Cc2ccc(OC)cc2)n(-c2ccccc2Cl)n1] | Methyl 1-(2- chlorophenyl)-5- (4- methoxybenzyl)- 1H-pyrazole-3- carboxylate | A | 10.11 | 357 |
| D3 | 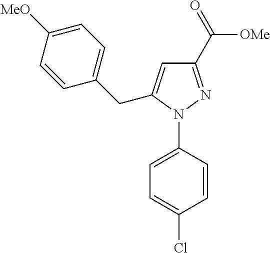 [SMILES: COC(=O)c1cc(Cc2ccc(OC)cc2)n(-c2ccc(Cl)cc2)n1] | Methyl 1-(4- chlorophenyl)-5- (4- methoxybenzyl)- 1H-pyrazole-3- carboxylate | A | 10.27 | 357 |
| D4 | 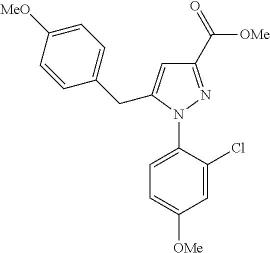 [SMILES: COC(=O)c1cc(Cc2ccc(OC)cc2)n(-c2ccc(OC)cc2Cl)n1] | Methyl 1-(2- chloro-4- methoxyphenyl)-5- (4- methoxybenzyl)- 1H-pyrazole-3- carboxylate | A | 10.20 | 387 |
| D5 | 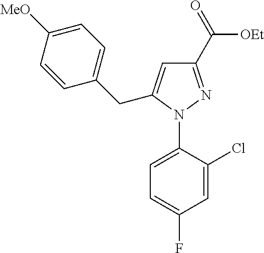 [SMILES: CCOC(=O)c1cc(Cc2ccc(OC)cc2)n(-c2ccc(F)cc2Cl)n1] | Ethyl 1-(2-chloro- 4-fluorophenyl)-5- (4- methoxybenzyl)- 1H-pyrazole-3- carboxylate | D | 11.00 | 389 |
| D6 | 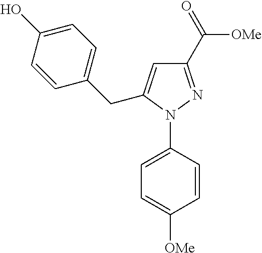 [SMILES: COC(=O)c1cc(Cc2ccc(O)cc2)n(-c2ccc(OC)cc2)n1] | Methyl 5-(4- hydroxybenzyl)-1- (4- methoxyphenyl)- 1H-pyrazole-3- carboxylate | B | 10.11 | 339 |
| D7 | 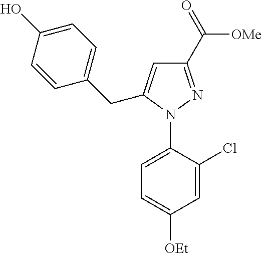 [SMILES: CCOc1ccc(-n2nc(C(=O)OC)cc2Cc2ccc(O)cc2)c(Cl)c1] | Methyl 1-(2- chloro-4- ethoxyphenyl)-5- (4-hydroxybenzyl)- 1H-pyrazole-3- carboxylate | B | 10.43 | 387 |
| D8 | 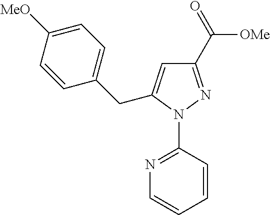 [SMILES: COC(=O)c1cc(Cc2ccc(OC)cc2)n(-c2ccccn2)n1] | Methyl 5-(4- methoxybenzyl)-1- (pyridin-2-yl)-1H- pyrazole-3- carboxylate | B | 10.55 | 324 |
| D9 | 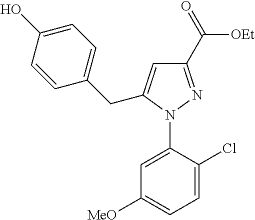 [SMILES: CCOC(=O)c1cc(Cc2ccc(O)cc2)n(-c2cc(OC)ccc2Cl)n1] | Ethyl 1-(2-chloro- 5-methoxyphenyl)- 5-(4- hydroxybenzyl)- 1H-pyrazole-3- carboxylate | B | 11.62 | 387 |
| D10 | 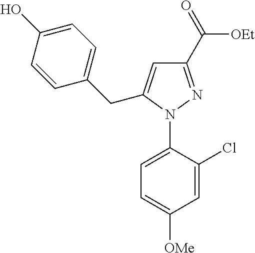 [SMILES: CCOC(=O)c1cc(Cc2ccc(O)cc2)n(-c2ccc(OC)cc2Cl)n1] | Ethyl 1-(2-chloro- 4-methoxyphenyl)- 5-(4- hydroxybenzyl)- 1H-pyrazole-3- carboxylate | B | 10.37 | 387 |
| D11 | 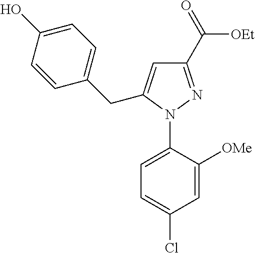 [SMILES: CCOC(=O)c1cc(Cc2ccc(O)cc2)n(-c2ccc(Cl)cc2OC)n1] | Ethyl 1-(4-chloro- 2-methoxyphenyl)- 5-(4- hydroxybenzyl)- 1H-pyrazole-3- carboxylate | B | 10.15 | 387 |
| D12 | 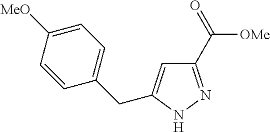 [SMILES: COC(=O)c1cc(Cc2ccc(OC)cc2)[nH]n1] | Methyl 5-(4- methoxybenzyl)- 1H-pyrazole-3- carboxylate | A | 8.73 | 247 |
| D13 | 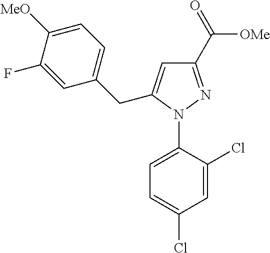 [SMILES: COC(=O)c1cc(Cc2ccc(OC)c(F)c2)n(-c2ccc(Cl)cc2Cl)n1] | Methyl 1-(2,4- dichlorophenyl)-5- (3-fluoro-4- methoxybenzyl)- 1H-pyrazole-3- carboxylate | A | 10.29 | 409 |
| D14 | 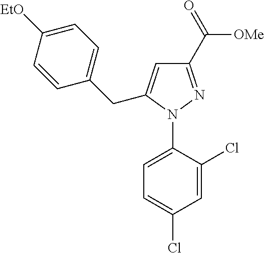 [SMILES: CCOc1ccc(Cc2cc(C(=O)OC)nn2-c2ccc(Cl)cc2Cl)cc1] | Methyl 1-(2,4- dichlorophenyl)-5- (4-ethoxybenzyl)- 1H-pyrazole-3- carboxylate | A | 10.74 | 405 |
| D15 | 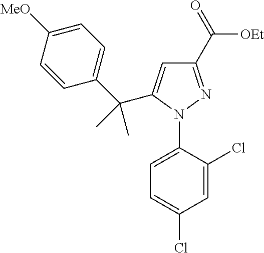 [SMILES: CCOC(=O)c1cc(C(C)(C)c2ccc(OC)cc2)n(-c2ccc(Cl)cc2Cl)n1] | Ethyl 1-(2,4- dichlorophenyl)-5- (2-(4- methoxyphenyl) propan-2-yl)-1H- pyrazole-3- carboxylate | B | 11.75 | 433 |
| D16 | 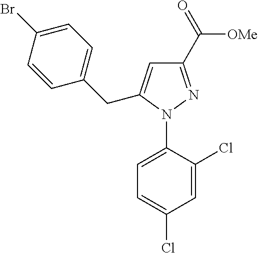 [SMILES: COC(=O)c1cc(Cc2ccc(Br)cc2)n(-c2ccc(Cl)cc2Cl)n1] | Methyl 5-(4- bromobenzyl)-1- (2,4- dichlorophenyl)- 1H-pyrazole-3- carboxylate | B | 11.59 | 441/443 |
| D17 | 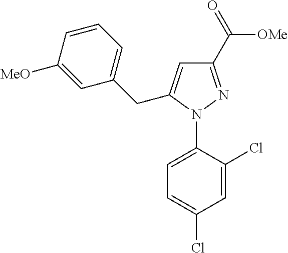 [SMILES: COC(=O)c1cc(Cc2cccc(OC)c2)n(-c2ccc(Cl)cc2Cl)n1] | Methyl 1-(2,4- dichlorophenyl)-5- (3- methoxybenzyl)- 1H-pyrazole-3- carboxylate | A | 10.48 | 391 |
| D18 | 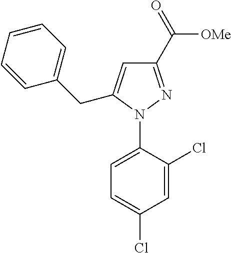 [SMILES: COC(=O)c1cc(Cc2ccccc2)n(-c2ccc(Cl)cc2Cl)n1] | Methyl 5-benzyl-1- (2,4- dichlorophenyl)- 1H-pyrazole-3- carboxylate | ¹H-NMR (CDCl₃, 300 MHz, δ)□: 7.53 (d, J = 2.2 Hz, 1H, ArH); 7.29-7.13 (m, 5H, ArH); 6.99 (m, 2H, ArH); 6.70 (s, 1H, ArH); 3.91 (s, 3H, OCH₃); 3.81 (br s, 2H, CH₂). | | |
| D19 | 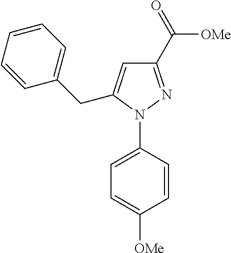 [SMILES: COC(=O)c1cc(Cc2ccccc2)n(-c2ccc(OC)cc2)n1] | Methyl 5-benzyl-1- (4- methoxyphenyl)- 1H-pyrazole-3- carboxylate | A | 10.35 | 323 |
| D20 | 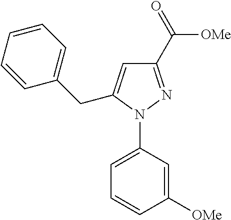 [SMILES: COC(=O)c1cc(Cc2ccccc2)n(-c2cccc(OC)c2)n1] | Methyl 5-benzyl-1- (3- methoxyphenyl)- 1H-pyrazole-3- carboxylate | A | 10.26 | 323 |

### Intermediate D21. Ethyl 1-(2-chloro-5-methoxyphenyl)-5-{4-[(tetrahydro-2H-pyran-2-yl)oxy]benzyl}-1H-pyrazole-3-carboxylate

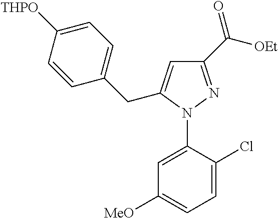

DHP (0.45 mL, 5.01 mmol) and PPTS (63 mg, 0.25 mmol) were added to a solution of ethyl 1-(2-chloro-5-methoxyphenyl)-5-(4-hydroxybenzyl)-1H-pyrazole-3-carboxylate (0.48 g, 1.25 mmol) in CH₂Cl₂ (10 mL) and the mixture was stirred at rt for 16 h. The reaction mixture was poured into water (20 mL) and the aqueous layer was extracted with CH₂Cl₂ (2×20 mL). The combined organic layers were dried over Na₂SO₄ (anhydrous), filtered and concentrated. The crude residue was purified by flash chromatography on SiO₂ (26% EtOAc/hexanes), to afford the title compound (yellow foam, 0.46 g, 77% yield).

HPLC-MS (Method B): Ret, 11.62 min; ESI⁺-MS m/z: 471 (M+1).

This method was used for the preparation of intermediates D22 and D23 using suitable starting materials:

| | | | | Ret | MS |
|---|---|---|---|---|---|
| INT | Structure | Chemical name | Method | (min) | (M + H) |
| D22 | 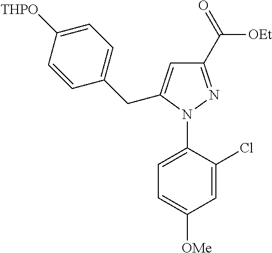 | Ethyl 1-(2-chloro- 4-methoxyphenyl)- 5-{4-[(tetrahydro- 2H-pyran-2- yl)oxy]benzyl}-1H- pyrazole-3- carboxylate | B | 11.62 | 471 |
| D23 | 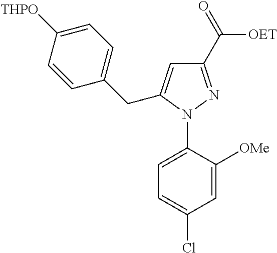 | Ethyl 1-(4-chloro- 2-methoxyphenyl)- 5-{4-[(tetrahydro- 2H-pyran-2- yl)oxy]benzyl}-1H- pyrazole-3- carboxylate | B | 11.60 | 471 |

### Intermediate D24. Methyl 1-isobutyl-5-(4-methoxybenzyl)-1H-pyrazole-3-carboxylate

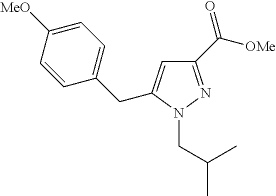 [SMILES: COC(=O)c1cc(Cc2ccc(OC)cc2)n(CC(C)C)n1]

t-BuOK (0.38 g, 3.38 mmol) and 1-iodo-2-methylpropane (0.52 mL, 4.50 mmol) were added to a solution of methyl 5-(4-methoxybenzyl)-1H-pyrazole-3-carboxylate (0.55 g, 2.25 mmol) in THF (15 mL) and the mixture was heated under reflux for 44 h. The reaction mixture was cooled down to rt, poured into water (20 mL) and extracted with EtOAc (2×20 mL). The organic layer was dried over Na₂SO₄ (anhydrous), filtered and concentrated. The crude residue was purified by flash chromatography on SiO₂ (15-32% EtOAc/hexanes), to afford the title compound (yellow foam, 63% yield).

HPLC-MS (Method A): Ret, 9.97 min; ESI⁺-MS m/z: 303 (M+1).

### Intermediate D25. Methyl 1-(4-chlorobenzyl)-5-(4-methoxybenzyl)-1H-pyrazole-3-carboxylate

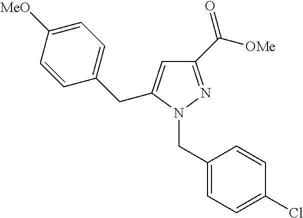 [SMILES: COC(=O)c1cc(Cc2ccc(OC)cc2)n(Cc2ccc(Cl)cc2)n1]

NaH (60% mineral oil suspension, 91 mg, 2.28 mmol) was added to a 0° C. cooled solution of methyl 5-(4-methoxybenzyl)-1H-pyrazole-3-carboxylate (0.51 g, 2.07 mmol) in THF (10 mL). The reaction mixture was allowed to warm to rt and stirred for 30 min. A solution of 1-(bromomethyl)-4-chlorobenzene (0.47 g, 2.28 mmol) was added and stirring continued for 3.5 h. The mixture was poured into water (10 mL) and the aqueous layer was extracted with EtOAc (2×20 mL). The combined organic layers were dried over anhydrous Na₂SO₄, filtered and concentrated. The crude residue was purified by flash chromatography on SiO₂ (26→40% EtOAc/hexanes), to afford the title compound (orange oil, 0.63 g, 83% yield).

HPLC-MS (Method A): Ret, 10.37 min; ESI⁺-MS m/z: 371 (M+1).

This method was used for the preparation of intermediates D26-D29 using suitable starting materials:

| | | | | Ret | MS |
|---|---|---|---|---|---|
| INT | Structure | Chemical name | Method | (min) | (M + H) |
| D26 | 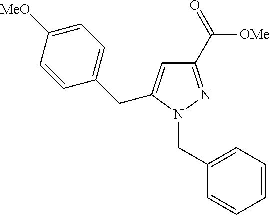 [SMILES: COC(=O)c1cc(Cc2ccc(OC)cc2)n(Cc2ccccc2)n1] | Methyl 1-benzyl-5- (4- methoxybenzyl)- 1H-pyrazole-3- carboxylate | A | 10.01 | 337 |
| D27 | 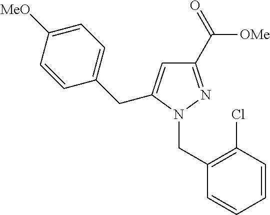 [SMILES: COC(=O)c1cc(Cc2ccc(OC)cc2)n(Cc2ccccc2Cl)n1] | Methyl 1-(2- chlorobenzyl)-5- (4- methoxybenzyl)- 1H-pyrazole-3- carboxylate | A | 10.35 | 371 |
| D28 | 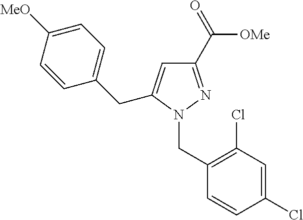 [SMILES: COC(=O)c1cc(Cc2ccc(OC)cc2)n(Cc2ccc(Cl)cc2Cl)n1] | Methyl 1-(2,4- dichlorobenzyl)-5- (4- methoxybenzyl)- 1H-pyrazole-3- carboxylate | A | 10.69 | 405 |
| D29 | 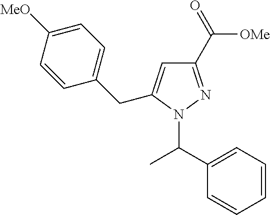 [SMILES: COC(=O)c1cc(Cc2ccc(OC)cc2)n(C(C)c2ccccc2)n1] | Methyl 5-(4- methoxybenzyl)-1- (1-phenylethyl)- 1H-pyrazole-3- carboxylate | A | 10.27 | 351 |

### Intermediate D30. Ethyl 1-(2,4-dichlorophenyl)-5-(4-methoxyphenoxy)-1H-pyrazole-3-carboxylate

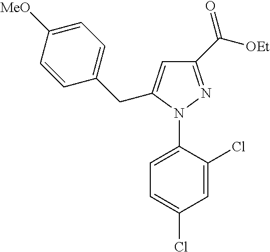 [SMILES: CCOC(=O)c1cc(Cc2ccc(OC)cc2)n(-c2ccc(Cl)cc2Cl)n1]

### a) Ethyl 1-(2,4-dichlorophenyl)-5-hydroxy-1H-pyrazole-3-carboxylate

K₂CO₃ (3.03 g, 21.93 mmol) was added to a solution of (2,4-dichlorophenyl)hydrazine hydrochloride (2.23 g, 10.44 mmol) in EtOH (10 mL). The mixture was stirred at rt for 10 minutes, dimethyl but-2-ynedioate (1.63 g, 11.49 mmol) was added and the reaction mixture was heated under reflux for 14 h. It was cooled down to −10° C. and water (12 mL) and HCl (10% aqueous solution, 12 mL) were slowly added. The mixture was allowed to reach rt and filtered. The solid was washed with water (3×20 mL) and dried under high vacuum to furnish the title compound (yellow solid, 2.50 g, 71% yield).

HPLC-MS (Method E): Ret, 10.59 min; ESI⁺-MS m/z: 301 (M+1).

### b) Title Compound

Cu(OAc)₂ (1.22 g, 6.71 mmol) and Py (0.81 mL, 10.06 mmol) were added to a solution of ethyl 1-(2,4-dichlorophenyl)-5-hydroxy-1H-pyrazole-3-carboxylate (1.01 g, 3.35 mmol) and 4-methoxyphenylboronic acid (0.76 g, 5.03 mmol) in CH₂Cl₂ (30 mL) and the mixture was stirred for 24 h in an open flask. Additional Cu(OAc)₂ (0.61 g, 3.35 mmol) and Py (0.41 mL, 5.03 mmol) were added and stirring continued for 24 h. NH₄Cl (saturated aqueous solution, 30 mL), NH₃ (33% aqueous solution, 3 mL) and CH₂Cl₂ (10 mL) were added and the mixture was stirred at rt until a solid appeared. The mixture was filtered and mother liquors were extracted with CH₂Cl₂ (2×20 mL), dried over anhydrous Na₂SO₄, filtered and concentrated. The crude residue was purified by flash chromatography on SiO₂ (10→32% EtOAc/hexanes) and by medium pressure flash chromatography (Combiflash, 0-10%; MeOH/CH₂Cl₂) to give the title compound (yellow oil, 180 mg, 13% yield).

HPLC-MS (Method A): Ret, 10.87 min; ESI⁺-MS m/z: 407 (M+1).

### Intermediate D31. Ethyl 1-(2,4-dichlorophenyl)-5-[4-(methylsulfonamido)phenoxy]-1H-pyrazole-3-carboxylate

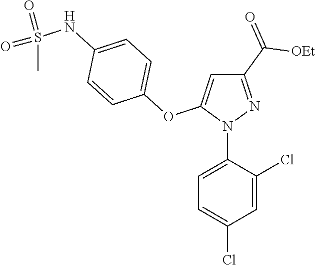 [SMILES: CCOC(=O)c1cc(Oc2ccc(NS(C)(=O)=O)cc2)n(-c2ccc(Cl)cc2Cl)n1]

### a) Ethyl 1-(2,4-dichlorophenyl)-5-(4-nitrophenoxy)-1H-pyrazole-3-carboxylate

K₂CO₃ (0.52 g, 3.74 mmol) and 1-fluoro-4-nitrobenzene (0.53 g, 3.74 mmol) were added to a solution of ethyl 1-(2,4-dichlorophenyl)-5-hydroxy-1H-pyrazole-3-carboxylate (0.75 g, 2.49 mmol) in DMF (12 mL) and the mixture was heated at 95° C. for 15 h. The reaction mixture was allowed to reach rt, poured into water (40 mL) and the aqueous layer was extracted with EtOAc (2×50 mL). The combined organic layers were dried over anhydrous Na₂SO₄, filtered and concentrated. The crude residue was purified by flash chromatography on SiO₂ (15% EtOAc/hexanes), to afford the title compound (orange foam, 0.38 g, 36% yield).

HPLC-MS (Method A): Ret, 10.71 min; ESI⁺-MS m/z: 422 (M+1).

### b) Ethyl 5-(4-aminophenoxy)-1-(2,4-dichlorophenyl)-1H-pyrazole-3-carboxylate

Fe (0.77 g, 13.86 mmol) was added to a suspension of ethyl 1-(2,4-dichlorophenyl)-5-(4-nitrophenoxy)-1H-pyrazole-3-carboxylate (0.59 g, 1.39 mmol) and NH₄Cl (0.30 g, 5.54 mmol) in water (7 mL) and MeOH (24 mL). The reaction mixture was warmed up to 50° C. and stirred at this temperature for 2 h. The mixture was cooled down to rt, filtered and rinsed with water (2×15 mL). The filtrate was extracted with EtOAc (2×50 mL); the combined organic layers were dried over Na₂SO₄ (anhydrous), filtered and concentrated. The crude residue was purified by flash chromatography on SiO₂ (10→60% EtOAc/hexanes) to afford the title compound (yellow foam, 71% 0.45 g, yield).

HPLC-MS (Method A): Ret, 10.03 min; ESI⁺-MS m/z: 392 (M+1).

### c) Title Compound

Methanesulfonyl chloride (96 μL, 1.23 mmol) was added to a 0° C. cooled solution of ethyl 5-(4-aminophenoxy)-1-(2,4-dichlorophenyl)-1H-pyrazole-3-carboxylate (440 mg, 1.12 mmol) and Py (180 μL, 2.24 mmol) in CH₂Cl₂ (40 mL). The reaction mixture was allowed to reach rt, stirred for 16 h and poured into water (20 mL). The organic layer was dried over Na₂SO₄ (anhydrous), filtered and concentrated. The crude residue was purified by flash chromatography on SiO₂ (0→2% MeOH/CH₂Cl₂) affording the title compound (yellow solid, 0.50 g, 89% yield).

HPLC-MS (Method A): Ret, 10.00 min; ESI⁺-MS m/z: 470 (M+1).

### Intermediate D32. Methyl 1-(2,4-dichlorophenyl)-5-[4-(methylsulfonamido)benzyl]-1H-pyrazole-3-carboxylate

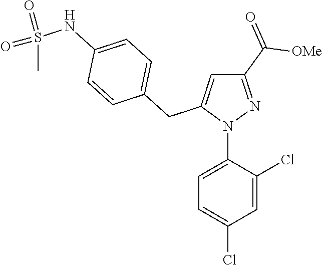 [SMILES: COC(=O)c1cc(Cc2ccc(NS(C)(=O)=O)cc2)n(-c2ccc(Cl)cc2Cl)n1]

### a) Methyl 5-{4-[(tert-butoxycarbonyl)amino]benzyl}-1-(2,4-dichlorophenyl)-1H-pyrazole-3-carboxylate

Pd(OAc)₂ (0.01 g, 0.05 mmol) was added to a degassed suspension of Cs₂CO₃ (0.67 g, 2.05 mmol), Xantphos (0.03 g, 0.05 mmol), tert-butyl carbamate (0.24 g, 2.05 mmol) and methyl 5-(4-bromobenzyl)-1-(2,4-dichlorophenyl)-1H-pyrazole-3-carboxylate (0.45 g, 1.02 mmol) in 1,4-dioxane (15 mL). The reaction was heated under reflux for 3.5 h and allowed to reach rt. Volatiles were removed by rotatory evaporation in the presence of SiO₂ and the residue was purified by flash chromatography on SiO₂ (30% EtOAc/hexanes) to afford the title compound (white solid, 0.36 g, 73% yield).

HPLC-MS (Method B): Ret, 11.38 min; ESI⁺-MS m/z: 476 (M+1).

### b) Methyl 5-(4-aminobenzyl)-1-(2,4-dichlorophenyl)-1H-pyrazole-3-carboxylate

TFA (2 mL) was added to a solution of the previous compound (0.35 g, 0.74 mmol) in CH₂Cl₂ (6 mL). The reaction mixture was stirred at rt until full conversion was achieved (1.5 h). The mixture was diluted with CH₂Cl₂ (50 mL) and was washed with K₂CO₃ (10% aqueous solution, 20 mL). The organic layer was dried over Na₂SO₄ (anhydrous), filtered and concentrated to afford the title compound (yellow solid, 0.26 g, 93% yield), which was used without further purification.

HPLC-MS (Method B): Ret, 10.31 min; ESI⁺-MS m/z: 376 (M+1).

### c) Title Compound

Methanesulfonyl chloride (62 μL, 0.80 mmol) was added to a 0° C. cooled solution of methyl 5-(4-aminobenzyl)-1-(2,4-dichlorophenyl)-1H-pyrazole-3-carboxylate (250 mg, 0.66 mmol) and Py (107 μL, 1.33 mmol) in CH₂Cl₂ (10 mL). The reaction mixture was allowed to reach rt and stirred for 18 h. Volatiles were removed by rotatory evaporation in the presence of SiO₂ and the residue was purified by flash chromatography on SiO₂ (20→40% EtOAc/hexanes) affording the title compound (white solid, 190 mg, 63% yield).

HPLC-MS (Method B): Ret, 10.19 min; ESI⁺-MS m/z: 454 (M+1).

### Intermediate E1. 1-(2,4-Dichlorophenyl)-5-(4-methoxybenzyl)-1H-pyrazole-3-carbaldehyde

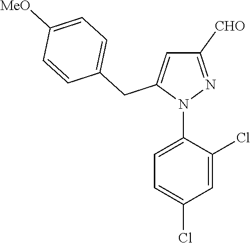 [SMILES: COc1ccc(Cc2cc(C=O)nn2-c2ccc(Cl)cc2Cl)cc1]

### a) [1-(2,4-Dichlorophenyl)-5-(4-methoxybenzyl)-1H-pyrazol-3-yl]methanol

LiBH₄ (2.0 M solution in THF, 7.13 mL, 14.26 mmol) was added dropwise to a 10° C. cooled solution of methyl 1-(2,4-dichlorophenyl)-5-(4-methoxybenzyl)-1H-pyrazole-3-carboxylate (2.79 g, 7.13 mmol) in Et₂O (30 mL) and MeOH (1 mL). The reaction mixture was allowed to reach rt and stirred for 2 h. The mixture was poured into water (30 mL) and stirred for 10 min; HCl (10% aqueous solution) was slowly added until pH=7 and the aqueous layer was extracted with EtOAc (2×100 mL). The combined organic layers were dried over Na₂SO₄ (anhydrous), filtered and concentrated. The crude residue was purified by flash chromatography on SiO₂ (2% MeOH/CH₂Cl₂) to afford the title compound (yellow oil, 2.39 g, 92% yield).

HPLC-MS (Method A): Ret, 9.93 min; ESI⁺-MS m/z: 363 (M+1).

### B) Title Compound

MnO₂ (88% purity, 1.06 g, 10.74 mmol) was added to a solution of the previous compound (0.39 g, 1.07 mmol) in CH₂Cl₂ (10 mL) and the mixture was heated under reflux for 16 h. The reaction mixture was allowed to reach rt, filtered through a pad of Celite, rinsed with CH₂Cl₂ (30 mL) and the solvent was concentrated off. The crude residue was purified by flash chromatography on SiO₂ (10→26% EtOAc/hexanes) to afford the title compound (yellow oil, 0.38 g, 99% yield).

HPLC-MS (Method A): Ret, 10.53 min; ESI⁺-MS m/z: 361 (M+1).

This method was used for the preparation of intermediates E2-E26 using suitable starting materials:

| | | | | Ret | MS |
|---|---|---|---|---|---|
| INT | Structure | Chemical name | Method | (min) | (M + H) |
| E2 | 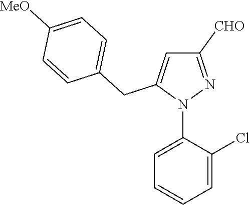 [SMILES: COc1ccc(Cc2cc(C=O)nn2-c2ccccc2Cl)cc1] | 1-(2- Chlorophenyl)-5- (4- methoxybenzyl)- 1H-pyrazole-3- carbaldehyde | A | 10.04 | 327 |
| E3 | 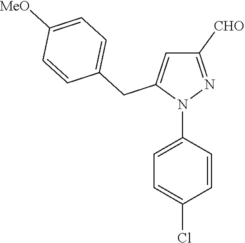 [SMILES: COc1ccc(Cc2cc(C=O)nn2-c2ccc(Cl)cc2)cc1] | 1-(4- Chlorophenyl)-5- (4- methoxybenzyl)- 1H-pyrazole-3- carbaldehyde | A | 10.25 | 327 |
| E4 | 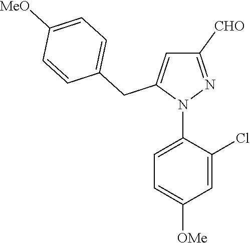 [SMILES: COc1ccc(Cc2cc(C=O)nn2-c2ccc(OC)cc2Cl)cc1] | 1-(2-Chloro-4- methoxyphenyl)-5- (4- methoxybenzyl)- 1H-pyrazole-3- carbaldehyde | A | 10.11 | 357 |
| E5 | 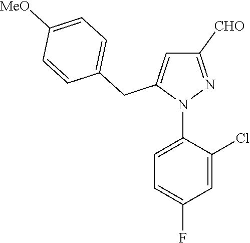 [SMILES: COc1ccc(Cc2cc(C=O)nn2-c2ccc(F)cc2Cl)cc1] | 1-(2-Chloro-4- fluorophenyl)-5-(4- methoxybenzyl)- 1H-pyrazole-3- carbaldehyde | D | 10.73 | 345 |
| E6 | 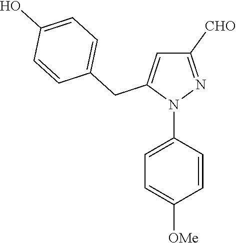 [SMILES: COc1ccc(-n2nc(C=O)cc2Cc2ccc(O)cc2)cc1] | 5-(4- Hydroxybenzyl)-1- (4- methoxyphenyl)- 1H-pyrazole-3- carbaldehyde | B | 9.75 | 309 |
| E7 | 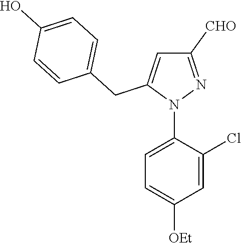 [SMILES: CCOc1ccc(-n2nc(C=O)cc2Cc2ccc(O)cc2)c(Cl)c1] | 1-(2-Chloro-4- ethoxyphenyl)-5- (4-hydroxybenzyl)- 1H-pyrazole-3- carbaldehyde | B | 10.48 | 357 |
| E8 | 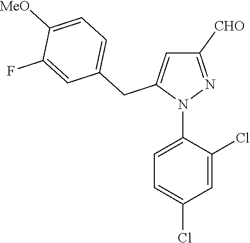 [SMILES: COc1ccc(Cc2cc(C=O)nn2-c2ccc(Cl)cc2Cl)cc1F] | 1-(2,4- Dichlorophenyl)-5- (3-fluoro-4- methoxybenzyl)- 1H-pyrazole-3- carbaldehyde | A | 10.34 | 379 |
| E9 | 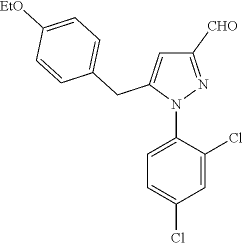 [SMILES: CCOc1ccc(Cc2cc(C=O)nn2-c2ccc(Cl)cc2Cl)cc1] | 1-(2,4- Dichlorophenyl)-5- (4-ethoxybenzyl)- 1H-pyrazole-3- carbaldehyde | A | 10.70 | 375 |
| E10 | 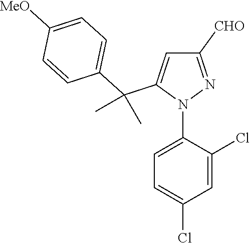 [SMILES: COc1ccc(C(C)(C)c2cc(C=O)nn2-c2ccc(Cl)cc2Cl)cc1] | 1-(2,4- Dichlorophenyl)-5- (2-(4- methoxyphenyl) propan-2-yl)-1H- pyrazole-3- carbaldehyde | B | 11.60 | 389 |
| E11 | 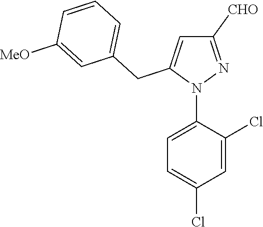 [SMILES: COc1cccc(Cc2cc(C=O)nn2-c2ccc(Cl)cc2Cl)c1] | 1-(2,4- Dichlorophenyl)-5- (3- methoxybenzyl)- 1H-pyrazole-3- carbaldehyde | A | 10.50 | 361 |
| E12 | 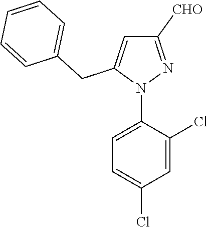 [SMILES: O=Cc1cc(Cc2ccccc2)n(-c2ccc(Cl)cc2Cl)n1] | 5-Benzyl-1-(2,4- dichlorophenyl)- 1H-pyrazole-3- carbaldehyde | A | 10.56 | 331 |
| E13 | 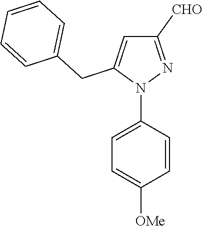 [SMILES: COc1ccc(-n2nc(C=O)cc2Cc2ccccc2)cc1] | 5-Benzyl-1-(4- methoxyphenyl) 1H-pyrazole-3- carbaldehyde | A | 9.99 | 293 |
| E14 | 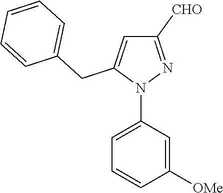 [SMILES: COc1cccc(-n2nc(C=O)cc2Cc2ccccc2)c1] | 5-Benzyl-1-(3- methoxyphenyl)- 1H-pyrazole-3- carbaldehyde | A | 10.02 | 293 |
| E15 | 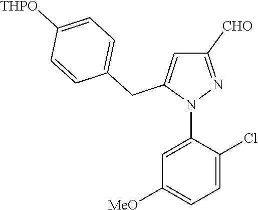 | 1-(2-Chloro-5- methoxyphenyl)-5- {4-[(tetrahydro-2H- pyran-2- yl)oxy]benzyl}-1H- pyrazole-3- carbaldehyde | B | 11.41 | 427 |
| E16 | 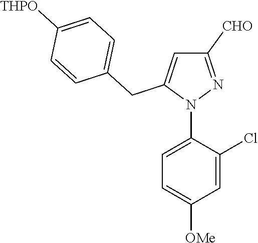 | 1-(2-Chloro-4- methoxyphenyl)-5- {4-[(tetrahydro-2H- pyran-2- yl)oxy]benzyl}-1H- pyrazole-3- carbaldehyde | B | 11.42 | 427 |
| E17 | 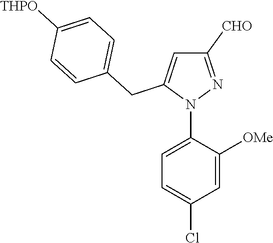 | 1-(4-Chloro-2- methoxyphenyl)-5- {4-[(tetrahydro-2H- pyran-2- yl)oxy]benzyl}-1H- pyrazole-3- carbaldehyde | B | 11.41 | 427 |
| E18 | 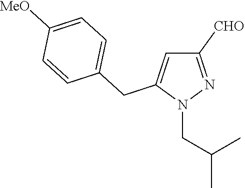 [SMILES: COc1ccc(Cc2cc(C=O)nn2CC(C)C)cc1] | 1-Isobutyl-5-(4- methoxybenzyl) 1H-pyrazole-3- carbaldehyde | A | 9.89 | 273 |
| E19 | 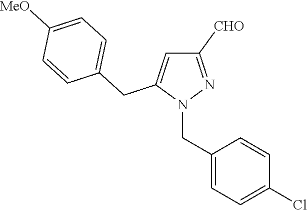 [SMILES: COc1ccc(Cc2cc(C=O)nn2Cc2ccc(Cl)cc2)cc1] | 1-(4- Chlorobenzyl)-5- (4- methoxybenzyl)- 1H-pyrazole-3- carbaldehyde | A | 10.33 | 341 |
| E20 | 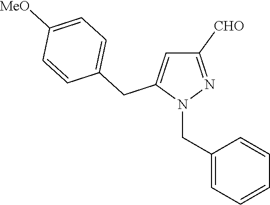 [SMILES: COc1ccc(Cc2cc(C=O)nn2Cc2ccccc2)cc1] | 1-Benzyl-5-(4- methoxybenzyl)- 1H-pyrazole-3- carbaldehyde | A | 9.90 | 307 |
| E21 | 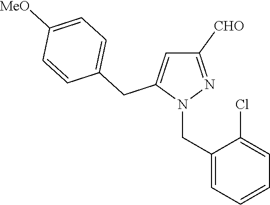 [SMILES: COc1ccc(Cc2cc(C=O)nn2Cc2ccccc2Cl)cc1] | 1-(2- Chlorobenzyl)-5- (4- methoxybenzyl)- 1H-pyrazole-3- carbaldehyde | A | 10.36 | 341 |
| E22 | 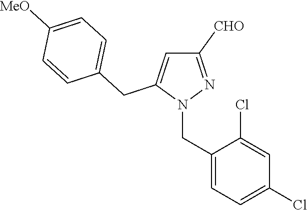 [SMILES: COc1ccc(Cc2cc(C=O)nn2Cc2ccc(Cl)cc2Cl)cc1] | 1-(2,4- Dichlorobenzyl)-5- (4- methoxybenzyl)- 1H-pyrazole-3- carbaldehyde | A | 10.70 | 375 |
| E23 | 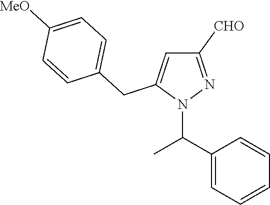 [SMILES: COc1ccc(Cc2cc(C=O)nn2C(C)c2ccccc2)cc1] | 5-(4- Methoxybenzyl)-1- (1-phenylethyl)- 1H-pyrazole-3- carbaldehyde | A | 10.26 | 321 |
| E24 | 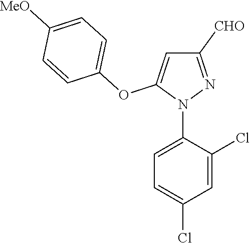 [SMILES: COc1ccc(Oc2cc(C=O)nn2-c2ccc(Cl)cc2Cl)cc1] | 1-(2,4- Dichlorophenyl)-5- (4- methoxyphenoxy)- 1H-pyrazole-3- carbaldehyde | A | 10.66 | 363 |
| E25 | 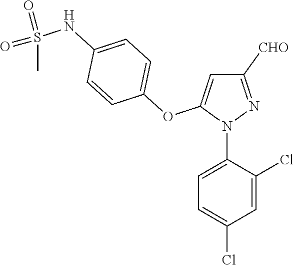 [SMILES: CS(=O)(=O)Nc1ccc(Oc2cc(C=O)nn2-c2ccc(Cl)cc2Cl)cc1] | N-{4-{[1-(2,4- Dichlorophenyl)-3- formyl-1H-pyrazol- 5- yl]oxy}phenyl) methanesulfonamide | A | 9.84 | 426 |
| E26 | 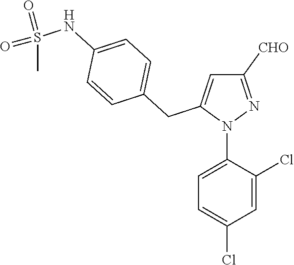 [SMILES: CS(=O)(=O)Nc1ccc(Cc2cc(C=O)nn2-c2ccc(Cl)cc2Cl)cc1] | N-(4-{[1-(2,4- dichlorophenyl)-3- formyl-1H-pyrazol- 5- yl]methyl}phenyl) methanesulfonamide | B | 10.26 | 424 |

### Intermediate E27. 5-(4-Methoxybenzyl)-1-phenyl-1H-pyrazole-3-carbaldehyde

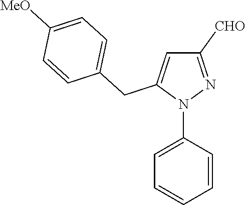 [SMILES: COc1ccc(Cc2cc(C=O)nn2-c2ccccc2)cc1]

### a) [5-(4-Methoxybenzyl)-1-phenyl-1H-pyrazol-3-yl]methanol

A suspension of [1-(2,4-dichlorophenyl)-5-(4-methoxybenzyl)-1H-pyrazol-3-yl]methanol (251 mg, 0.69 mmol, obtained in step a, intermediate E1), Et₃N (96 μL, 0.69 mmol) and Pd/C (10% w/w palladium on activated carbon, 313 mg, 0.14 mmol) in EtOH (10 mL) was stirred under H₂ atmosphere (balloon) for 3.5 days. The reaction mixture was filtered through Celite, washed with EtOH (2×10 mL), and concentrated. The crude residue was purified by flash chromatography on SiO₂ (3→5% MeOH/CH₂Cl₂), to afford the title compound (brown oil, 169 mg, 83% yield).

HPLC-MS (Method A): Ret, 9.22 min; ESI⁺-MS m/z: 295 (M+1).

### b) Title Compound

The title compound was obtained following the procedure described in intermediate E1, step b, and the previous compound as starting material.

HPLC-MS (Method A): Ret, 9.90 min; ESI⁺-MS m/z: 293 (M+1).

### Example 1. [1-(2,4-Dichlorophenyl)-5-(4-methoxybenzyl)-1H-pyrazol-3yl]methanamine

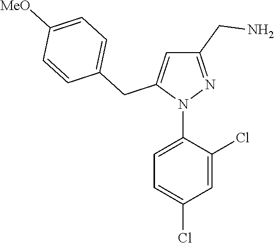 [SMILES: COc1ccc(Cc2cc(CN)nn2-c2ccc(Cl)cc2Cl)cc1]

### a) 1-(2,4-Dichlorophenyl)-5-(4-methoxybenzyl)-1H-pyrazole-3-carbaldehyde oxime

NH₂OH.HCl (43 mg, 0.62 mmol) was added to a solution of 1-(2,4-dichlorophenyl)-5-(4-methoxybenzyl)-1H-pyrazole-3-carbaldehyde (150 mg, 0.42 mmol) and Et₃N (87 μL, 0.62 mmol) in CH₂Cl₂ (10 mL). The reaction mixture was stirred at rt for 15.5 h, poured into water (10 mL) and extracted with CH₂Cl₂ (2×10 mL). The combined organic layers were dried over Na₂SO₄ (anhydrous), filtered and concentrated, to afford the title compound (yellow oil, 155 mg, 99% yield). This oxime was submitted to next step without further purification.

HPLC-MS (Method A): Ret, 13.19 min; ESI⁺-MS m/z: 376 (M+1).

### b) [1-(2,4-dichlorophenyl)-5-(4-methoxybenzyl)-1H-pyrazol-3-yl]methanamine

Zn dust (52 mg, 0.80 mmol) was added to a solution of the previous compound (150 mg, 0.40 mmol) in AcOH (5 mL) and the mixture was stirred at rt for 3 h. Additional Zn (52 mg, 0.80 mmol) was added and the resulting suspension was stirred until full conversion was achieved (15 h, TLC monitoring). The reaction mixture was filtered, rinsed with EtOAc (3×15 mL) and the solvent was concentrated off. The crude residue was poured into EtOAc (20 mL) and was washed with NaOH (10% aqueous solution, 15 mL). The organic layer was dried over Na₂SO₄ (anhydrous), filtered and concentrated. The crude residue was purified by flash chromatography on SiO₂ (CH₂Cl₂/MeOH/NH₄OH 95:5:0→85:15:1), to afford the title compound (yellow oil, 118 mg, 82% yield).

HPLC-MS (Method F): Ret, 17.41 min; ESI⁺-MS m/z: 362 (M+1).

This method was used for the preparation of examples 2-19 using suitable starting materials:

| | | | | Ret | MS |
|---|---|---|---|---|---|
| EX | Structure | Chemical name | Method | (min) | (M + H) |
| 2 | 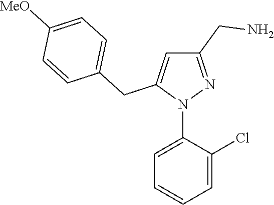 [SMILES: COc1ccc(Cc2cc(CN)nn2-c2ccccc2Cl)cc1] | [1-(2- Chlorophenyl)-5- (4- methoxybenzyl)- 1H-pyrazol-3- yl]methanamine | G | 16.10 | 328 |
| 3 | 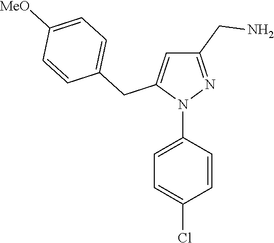 [SMILES: COc1ccc(Cc2cc(CN)nn2-c2ccc(Cl)cc2)cc1] | [1-(4- Chlorophenyl)-5- (4- methoxybenzyl)- 1H-pyrazol-3- yl]methanamine | F | 17.02 | 328 |
| 4 | 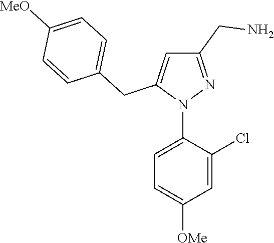 [SMILES: COc1ccc(Cc2cc(CN)nn2-c2ccc(OC)cc2Cl)cc1] | [1-(2-Chloro-4- methoxyphenyl)-5- (4- methoxybenzyl)- 1H-pyrazol-3- yl]methanamine | F | 17.01 | 358 |
| 5 | 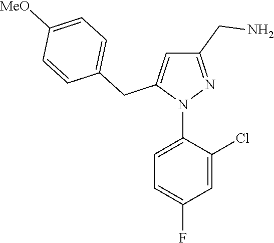 [SMILES: COc1ccc(Cc2cc(CN)nn2-c2ccc(F)cc2Cl)cc1] | [1-(2-Chloro-4- fluorophenyl)-5-(4- methoxybenzyl)- 1H-pyrazol-3- yl]methanamine | F | 16.08 | 346 |
| 6 | 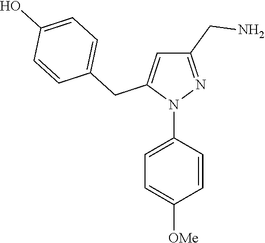 [SMILES: COc1ccc(-n2nc(CN)cc2Cc2ccc(O)cc2)cc1] | 4-{[3- (Aminomethyl)-1- (4- methoxyphenyl)- 1H-pyrazol-5- yl]methyl}phenol | F | 13.96 | 310 |
| 7 | 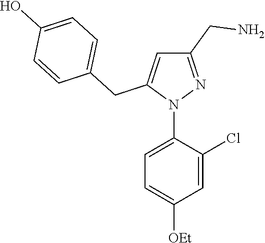 [SMILES: CCOc1ccc(-n2nc(CN)cc2Cc2ccc(O)cc2)c(Cl)c1] | 4-{[3- (Aminomethyl)-1- (2-chloro-4- ethoxyphenyl)-1H- pyrazol-5- yl]methyl}phenol | F | 15.66 | 358 |
| 8 | 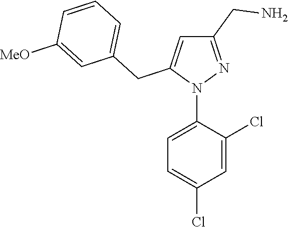 [SMILES: COc1cccc(Cc2cc(CN)nn2-c2ccc(Cl)cc2Cl)c1] | [1-(2,4- Dichlorophenyl)-5- (3- methoxybenzyl)- 1H-pyrazol-3- yl]methanamine | F | 17.30 | 362 |
| 9 | 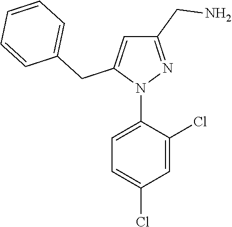 [SMILES: NCc1cc(Cc2ccccc2)n(-c2ccc(Cl)cc2Cl)n1] | [5-Benzyl-1-(2,4- dichlorophenyl)- 1H-pyrazol-3- yl]methanamine | F | 17.31 | 332 |
| 10 | 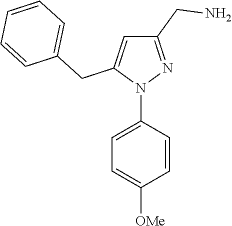 [SMILES: COc1ccc(-n2nc(CN)cc2Cc2ccccc2)cc1] | [5-Benzyl-1-(4- methoxyphenyl)- 1H-pyrazol-3- yl]methanamine | F | 15.64 | 294 |
| 11 | 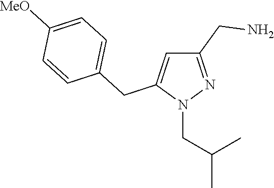 [SMILES: COc1ccc(Cc2cc(CN)nn2CC(C)C)cc1] | [1-Isobutyl-5-(4- methoxybenzyl)- 1H-pyrazol-3- yl]methanamine | F | 15.86 | 274 |
| 12 | 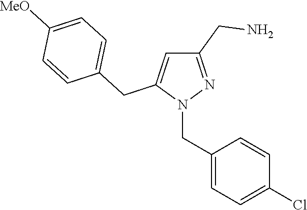 [SMILES: COc1ccc(Cc2cc(CN)nn2Cc2ccc(Cl)cc2)cc1] | [1-(4- Chlorobenzyl)-5- (4- methoxybenzyl)- 1H-pyrazol-3- yl]methanamine | F | 16.66 | 342 |
| 13 | 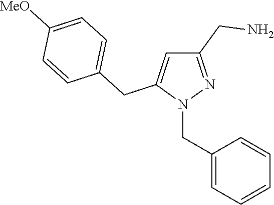 [SMILES: COc1ccc(Cc2cc(CN)nn2Cc2ccccc2)cc1] | [1-Benzyl-5-(4- methoxybenzyl)- 1H-pyrazol-3- yl]methanamine | F | 15.68 | 308 |
| 14 | 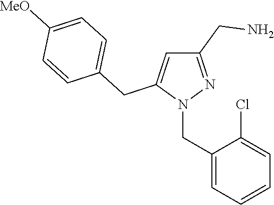 [SMILES: COc1ccc(Cc2cc(CN)nn2Cc2ccccc2Cl)cc1] | [1-(2- Chlorobenzyl)-5- (4- methoxybenzyl)- 1H-pyrazol-3- yl]methanamine | F | 16.69 | 342 |
| 15 | 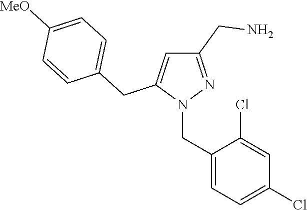 [SMILES: COc1ccc(Cc2cc(CN)nn2Cc2ccc(Cl)cc2Cl)cc1] | [1-(2,4- Dichlorobenzyl)-5- (4- methoxybenzyl)- 1H-pyrazol-3- yl]methanamine | F | 17.61 | 376 |
| 16 | 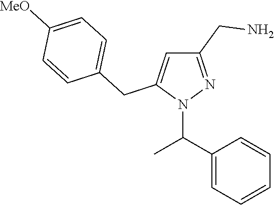 [SMILES: COc1ccc(Cc2cc(CN)nn2C(C)c2ccccc2)cc1] | [5-(4- Methoxybenzyl)-1- (1-phenylethyl)- 1H-pyrazol-3- yl]methanamine | F | 16.20 | 322 |
| 17 | 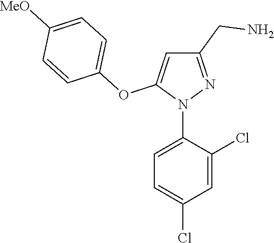 [SMILES: COc1ccc(Oc2cc(CN)nn2-c2ccc(Cl)cc2Cl)cc1] | [1-(2,4- Dichlorophenyl)-5- (4- methoxyphenoxy)- 1H-pyrazol-3- yl]methanamine | F | 18.11 | 364 |
| 18 | 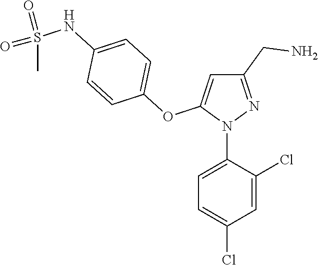 [SMILES: CS(=O)(=O)Nc1ccc(Oc2cc(CN)nn2-c2ccc(Cl)cc2Cl)cc1] | N-(4-{[3- (Aminomethyl)-1- (2,4- dichlorophenyl)- 1H-pyrazol-5- yl]oxy}phenyl) methanesulfonamide | F | 16.13 | 427 |
| 19 | 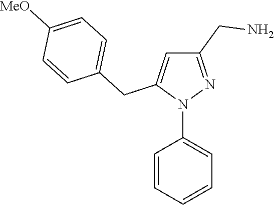 [SMILES: COc1ccc(Cc2cc(CN)nn2-c2ccccc2)cc1] | [5-(4- Methoxybenzyl)-1- phenyl-1H- pyrazol-3- yl]methanamine | F | 15.50 | 294 |

The same method was used for the preparation of intermediate F1 using suitable starting materials:

| | | | | Ret | MS |
|---|---|---|---|---|---|
| INT | Structure | Chemical name | Method | (min) | (M + H) |
| F1 | 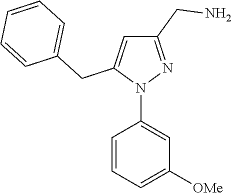 [SMILES: COc1cccc(-n2nc(CN)cc2Cc2ccccc2)c1] | [5-Benzyl-1-(3- methoxyphenyl)- 1H-pyrazol-3- yl]methanamine | A | 8.93 | 294 |

### Example 20. [5-(4-Methoxybenzyl)-1-(pyridin-2-yl)-1H-pyrazol-3-yl]methanamine

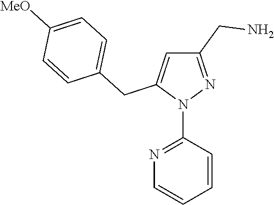 [SMILES: COc1ccc(Cc2cc(CN)nn2-c2ccccn2)cc1]

### a) [5-(4-Methoxybenzyl)-1-(pyridin-2-yl)-1H-pyrazol-3-yl]methanol

The title compound was obtained following the procedure described in intermediate E1, and using methyl 5-(4-methoxybenzyl)-1-(pyridin-2-yl)-1H-pyrazole-3-carboxylate as starting material.

HPLC-MS (Method B): Ret, 9.61 min; ESI⁺-MS m/z: 296 (M+1).

### b) Title Compound

Methanesulfonyl chloride (84 μL, 1.08 mmol) was added to a 0° C. cooled solution of the previous compound (290 mg, 0.98 mmol) and Et₃N (178 μL, 1.27 mmol) in CH₂Cl₂ (12 mL). The reaction mixture was stirred at rt for 2.5 h, poured into water (10 mL) and extracted with CH₂Cl₂ (2×10 mL). The combined organic layers were dried over Na₂SO₄ (anhydrous), filtered and concentrated, to afford 0.34 g of the corresponding methanesulfonate (light green oil). This oil was dissolved in DMF (10 mL) and stirred at rt for 18 h in the presence of NaN₃ (67 mg, 1.02 mmol). The mixture was poured into water (30 mL) and extracted with CH₂Cl₂ (2×20 mL). The combined organic layers were dried over Na₂SO₄ (anhydrous), filtered and concentrated, rendering 0.27 g of azido derivative as a brown oil. Finally, SnC₂.H₂O (0.37 g, 1.65 mmol) was added to an ethanolic (10 mL) solution of the azido substrate, stirring at rt for 18 h. Volatiles were removed by rotatory evaporation in the presence of SiO₂ and the residue was purified by flash chromatography on SiO₂ (CH₂Cl₂/MeOH/NH₄OH 98:2:1→96:4:1) to afford the title compound (yellow oil, 0.18 g, 65% yield).

HPLC-MS (Method F): Ret, 15.61 min; ESI⁺-MS m/z: 295 (M+1).

### Example 21. 1-[1-(2,4-Dichlorophenyl)-5-(4-methoxybenzyl)-1H-pyrazol-3-yl]-N,N-dimethylmethanamine

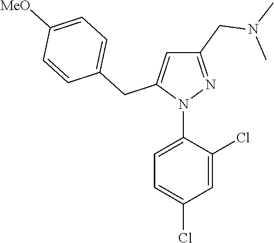 [SMILES: COc1ccc(Cc2cc(CN(C)C)nn2-c2ccc(Cl)cc2Cl)cc1]

N,N-Dimethylamine hydrochloride (26 mg, 0.33 mmol) and AcOH (0.40 mL) were added to a solution of 1-(2,4-dichlorophenyl)-5-(4-methoxybenzyl)-1H-pyrazole-3-carbaldehyde (78 mg, 0.22 mmol) in CH₂Cl₂ (4 mL). The reaction mixture was stirred at rt for 20 min and NaBH(OAc)₃ (92 mg, 0.43 mmol) was added. After 3.5 h, the mixture was poured into NaHCO₃ (saturated aqueous solution, 10 mL) and extracted with CH₂Cl₂ (2×10 mL); the combined organic layers were dried over Na₂SO₄ (anhydrous), filtered and concentrated. The crude residue was purified by flash chromatography on SiO₂ (CH₂Cl₂/MeOH/NH₄OH 95:5:1→90:10:1) to afford the title compound (yellow oil, 21 mg, 25% yield).

HPLC-MS (Method F): Ret, 18.59 min; ESI⁺-MS m/z: 390 (M+1).

This method was used for the preparation of examples 22 and 23 using suitable starting materials:

| | | | | Ret | MS |
|---|---|---|---|---|---|
| Ex | Structure | Chemical name | Method | (min) | (M + H) |
| 22 | 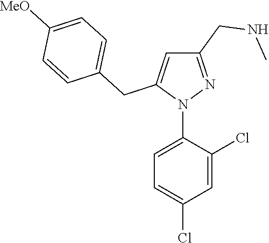 [SMILES: CNCc1cc(Cc2ccc(OC)cc2)n(-c2ccc(Cl)cc2Cl)n1] | 1-[1-{2,4- Dichlorophenyl)-5- (4- methoxybenzyl)- 1H-pyrazol-3-yl]- N- methylmethanamine | F | 17.42 | 376 |
| 23 | 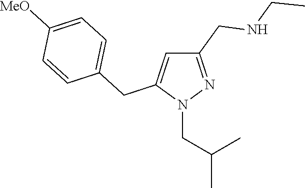 [SMILES: CCNCc1cc(Cc2ccc(OC)cc2)n(CC(C)C)n1] | N-{[1-Isobutyl-5- (4- methoxybenzyl)- 1H-pyrazol-3- yl]methyl} ethanamine | F | 16.28 | 302 |

### Example 24. 1-[1-(2,4-Dichlorophenyl)-5-(4-methoxybenzyl)-1H-pyrazol-3-yl]ethanamine

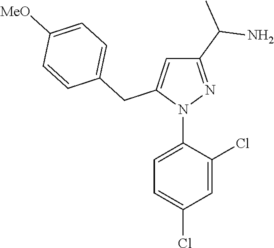 [SMILES: COc1ccc(Cc2cc(C(C)N)nn2-c2ccc(Cl)cc2Cl)cc1]

### a) N-{[1-(2,4-Dichlorophenyl)-5-(4-methoxybenzyl)-1H-pyrazol-3-yl]methylene}-2-methylpropane-2-sulfinamide

A mixture of 1-(2,4-dichlorophenyl)-5-(4-methoxybenzyl)-1H-pyrazole-3-carbaldehyde (4.90 g, 13.57 mmol), 2-methylpropane-2-sulfinamide (1.81 g, 14.93 mmol) and Cs₂CO₃ (6.98 g, 17.64 mmol) in CH₂Cl₂ (100 mL) was heated under reflux for 19 h. The reaction mixture was allowed to reach rt and was washed with brine (80 mL). The aqueous layer was extracted with CH₂Cl₂ (60 mL) and the combined organic layers were dried over Na₂SO₄ (anhydrous), filtered and concentrated, rendering the title compound (yellow foam, 6.68 g, quantitative yield) which was submitted to the next step without further purification.

HPLC-MS (Method A): Ret, 11.09 min; ESI⁺-MS m/z: 464 (M+1).

### b) N-{1-[1-(2,4-Dichlorophenyl)-5-(4-methoxybenzyl)-1H-pyrazol-3-yl]ethyl}-2-methylpropane-2-sulfinamide

MeMgBr (3.0 M solution in Et₂O, 10 mL, 30.0 mmol) was added to a 0° C. cooled solution of the previous compound (6.68 g, 13.57 mmol) in CH₂Cl₂ (80 mL) and stirred for 6 h at 0° C. The mixture was poured into NH₄Cl (saturated aqueous solution, 100 mL) and the aqueous layer was extracted with CH₂Cl₂ (2×100 mL). The combined organic layers were dried over Na₂SO₄ (anhydrous), filtered and concentrated, to afford the title compound (yellow foam, 6.12 g, 94% yield) which was submitted to the next step without further purification.

HPLC-MS (Method A): Ret, 10.70 min and 10.77 min; ESI⁺-MS m/z: 480 (M+1).

### c) Title Compound

HCl (21.0 mL, 1.25 M solution in MeOH, 26.25 mmol) was added to a solution of the previous compound (6.12 g, 12.75 mmol) in MeOH (70 mL) and the mixture was stirred at rt for 15 h. More HCl (10.2 mL, 12.75 mmol) was added and stirring continued until completion of the reaction (23 h, TLC monitoring). The solvent was concentrated off, the residue was dissolved in CH₂Cl₂ (80 mL) and was washed with NaOH (10% aqueous solution, 70 mL). The aqueous layer was extracted with CH₂Cl₂ (2×50 mL) and the combined organic layers were dried over Na₂SO₄ (anhydrous), filtered and concentrated. The crude residue was purified by flash chromatography on SiO₂ (CH₂Cl₂/MeOH/NH₄OH 95:5:1→90:10:1) to afford the title compound (yellow oil, 4.07 g, 85% yield).

HPLC-MS (Method F): Ret, 17.78 min; ESI⁺-MS m/z: 376 (M+1).

This method was used for the preparation of examples 25-33 using suitable starting materials:

| | | | | Ret | MS |
|---|---|---|---|---|---|
| Ex | Structure | Chemical name | Method | (min) | (M + H) |
| 25 | 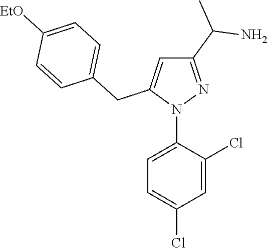 [SMILES: CCOc1ccc(Cc2cc(C(C)N)nn2-c2ccc(Cl)cc2Cl)cc1] | 1-[1-(2,4- Dichlorophenyl)-5- (4-ethoxybenzyl)- 1H-pyrazol-3- yl]ethanamine | F | 18.21 | 390 |
| 26 | 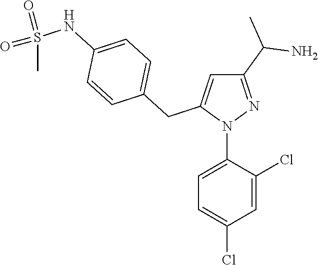 [SMILES: CC(N)c1cc(Cc2ccc(NS(C)(=O)=O)cc2)n(-c2ccc(Cl)cc2Cl)n1] | N-(4-{[3-(1- Aminoethyl)-1- (2,4- dichlorophenyl)- 1H-pyrazol-5- yl]methyl}phenyl) methanesulfonamide | F | 15.75 | 439 |
| 27 | 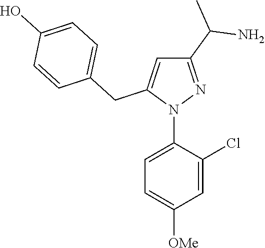 [SMILES: COc1ccc(-n2nc(C(C)N)cc2Cc2ccc(O)cc2)c(Cl)c1] | 4-{[3-(1- Aminoethyl)-1-(2- chloro-4- methoxyphenyl)- 1H-pyrazol-5- yl]methyl}phenol | F | 15.43 | 358 |
| 28 | 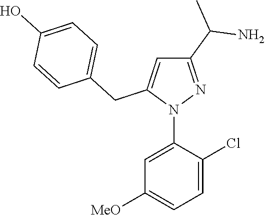 [SMILES: COc1ccc(Cl)c(-n2nc(C(C)N)cc2Cc2ccc(O)cc2)c1] | 4-{[3-(1- Aminoethyl)-1-(2- chloro-5- methoxyphenyl)- 1H-pyrazol-5- yl]methyl}phenol | F | 15.30 | 358 |
| 29 | 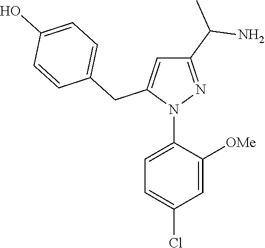 [SMILES: COc1cc(Cl)ccc1-n1nc(C(C)N)cc1Cc1ccc(O)cc1] | 4-{[3-(1- Aminoethyl)-1-(4- chloro-2- methoxyphenyl)- 1H-pyrazol-5- yl]methyl}phenol | F | 15.50 | 358 |
| 30 | 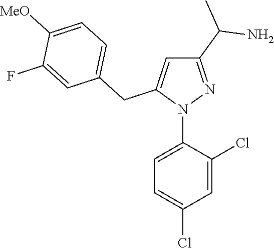 [SMILES: COc1ccc(Cc2cc(C(C)N)nn2-c2ccc(Cl)cc2Cl)cc1F] | 1-[1-(2,4- Dichlorophenyl)-5- (3-fluoro-4- methoxybenzyl)- 1H-pyrazol-3- yl)]ethanamine | A | 9.75 | 394 |
| 31 | 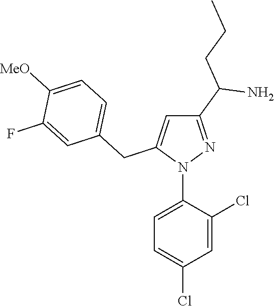 [SMILES: CCCC(N)c1cc(Cc2ccc(OC)c(F)c2)n(-c2ccc(Cl)cc2Cl)n1] | 1-[1-(2,4- Dichlorophenyl)-5- (3-fluoro-4- methoxybenzyl)- 1H-pyrazol-3- yl]butan-1-amine | D | 10.31 | 422 |
| 32 | 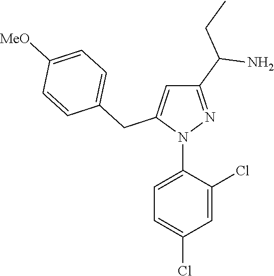 [SMILES: CCC(N)c1cc(Cc2ccc(OC)cc2)n(-c2ccc(Cl)cc2Cl)n1] | 1-[1-(2,4- Dichlorophenyl)-5- (4- methoxybenzyl)- 1H-pyrazol-3- yl]propan-1-amine | B | 10.40 | 390 |
| 33 | 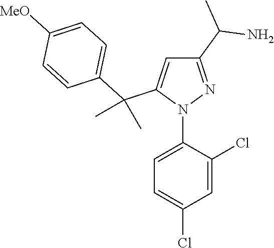 [SMILES: COc1ccc(C(C)(C)c2cc(C(C)N)nn2-c2ccc(Cl)cc2Cl)cc1] | 1-[1-(2,4- Dichlorophenyl)-5- (2-(4- methoxyphenyl) propan-2-yl)-1H- pyrazol-3- yl]ethanamine | B | 10.63 | 404 |

### Example 34. 3-{[3-(Aminomethyl)-1-(2,4-dichlorophenyl)-1H-pyrazol-5-yl]methyl}phenol

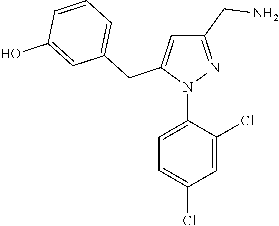 [SMILES: NCc1cc(Cc2cccc(O)c2)n(-c2ccc(Cl)cc2Cl)n1]

BBr₃ (1.0 M in CH₂Cl₂, 2.10 mL, 2.10 mmol) was added to a 0° C. cooled solution of [1-(2,4-dichlorophenyl)-5-(3-methoxybenzyl)-1H-pyrazol-3-yl]methanamine (257 mg, 0.71 mmol) in CH₂Cl₂ (12 mL). The reaction mixture was allowed to reach rt and stirred for 4 h. The reaction mixture was washed with NaHCO₃ (saturated aqueous solution, 8 mL). The aqueous layer was extracted with MeOH/CH₂Cl₂ (5%, 4×10 mL). The combined organic layers were dried over Na₂SO₄ (anhydrous), filtered and concentrated. The crude residue was purified by flash chromatography on SiO₂ (CH₂Cl₂/MeOH/NH₄OH 95:5:1→90:10:1), to afford the title compound (pale yellow oil, 89 mg, 36% yield).

HPLC-MS (Method F): Ret, 15.70 min; ESI⁺-MS m/z: 348 (M+1).

This method was used for the preparation of examples 35-37 using suitable starting materials:

| | | | | Ret | MS |
|---|---|---|---|---|---|
| Ex | Structure | Chemical name | Method | (min) | (M + H) |
| 35 | 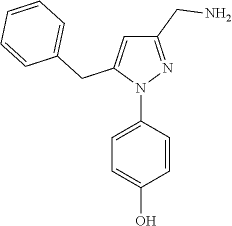 [SMILES: NCc1cc(Cc2ccccc2)n(-c2ccc(O)cc2)n1] | 4-[3- (Aminomethyl)-5- benzyl-1H- pyrazol-1- yl]phenol | F | 13.85 | 280 |
| 36 | 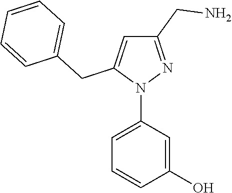 [SMILES: NCc1cc(Cc2ccccc2)n(-c2cccc(O)c2)n1] | 3-[3- (Aminomethyl)-5- benzyl-1H- pyrazol-1- yl]phenol | F | 14.35 | 280 |
| 37 | 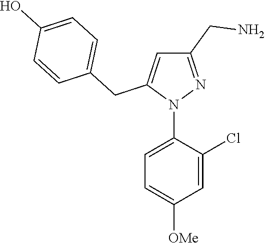 [SMILES: COc1ccc(-n2nc(CN)cc2Cc2ccc(O)cc2)c(Cl)c1] | 4-{[3- (Aminomethyl)-1- (2-chloro-4- methoxyphenyl)- 1H-pyrazol-5- yl]methyl}phenol | H | 15.42 | 344 |

### Example 38. 4-{[3-(Aminomethyl)-1-(2,4-dichlorophenyl)-1H-pyrazol-5-yl]methyl}phenol

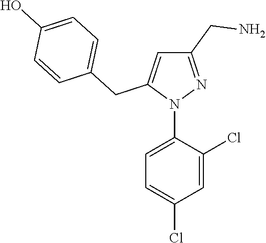 [SMILES: NCc1cc(Cc2ccc(O)cc2)n(-c2ccc(Cl)cc2Cl)n1]

A suspension of [1-(2,4-dichlorophenyl)-5-(4-methoxybenzyl)-1H-pyrazol-3 yl]methanamine (965 mg, 2.46 mmol) in HBr (48% aqueous solution, 10 mL) was heated under reflux for 2.5 h. The mixture was allowed to reach rt and the solvent was concentrated off. The residue was poured into CH₂Cl₂ (10 mL) and the organic layer was washed with NaHCO₃ (saturated aqueous solution, 5 mL). The aqueous layer was extracted with MeOH/CH₂Cl₂ (˜2%, 2×10 mL). The combined organic layers were washed with NaHCO₃ (saturated aqueous solution, 2×10 mL) and with water (10 mL), dried over Na₂SO₄ (anhydrous), filtered and concentrated. The crude residue was purified by flash chromatography on SiO₂ (CH₂Cl₂/MeOH/NH₄OH 90:10:1), followed by medium pressure flash chromatography (Combiflash, 0→100% water/MeOH), to afford the title compound (white solid, 377 mg, 44% yield).

HPLC-MS (Method F): Ret, 15.43 min; ESI⁺-MS m/z: 348 (M+1).

This method was used for the preparation of examples 39-55 using suitable starting materials:

| | | | | Ret | MS |
|---|---|---|---|---|---|
| Ex | Structure | Chemical name | Method | (min) | (M + H) |
| 39 | 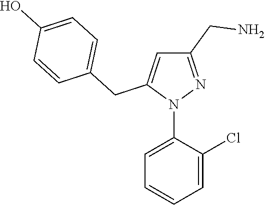 [SMILES: NCc1cc(Cc2ccc(O)cc2)n(-c2ccccc2Cl)n1] | 4-{[3- (Aminomethyl)-1- (2-chlorophenyl)- 1H-pyrazol-5- yl]methyl}phenol | F | 14.29 | 314 |
| 40 | 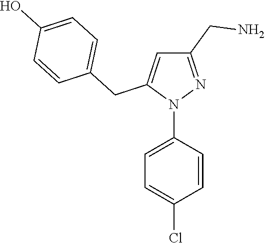 [SMILES: NCc1cc(Cc2ccc(O)cc2)n(-c2ccc(Cl)cc2)n1] | 4-{[3- (Aminomethyl)-1- (4-chlorophenyl)- 1H-pyrazol-5- yl]methyl}phenol | F | 15.13 | 314 |
| 41 | 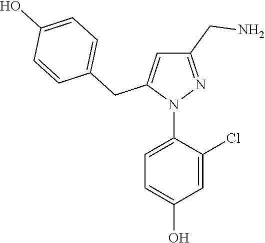 [SMILES: NCc1cc(Cc2ccc(O)cc2)n(-c2ccc(O)cc2Cl)n1] | 4-[3- (Aminomethyl)-5- (4-hydroxybenzyl)- 1H-pyrazol-1-yl]-3- chlorophenol | F | 13.56 | 330 |
| 42 | 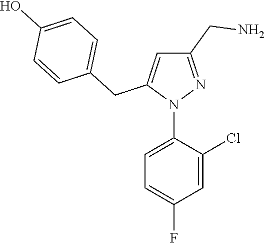 [SMILES: NCc1cc(Cc2ccc(O)cc2)n(-c2ccc(F)cc2Cl)n1] | 4-{[3- (Aminomethyl)-1- (2-chloro-4- fluorophenyl)-1H- pyrazol-5- yl]methyl}phenol | F | 14.24 | 332 |
| 43 | 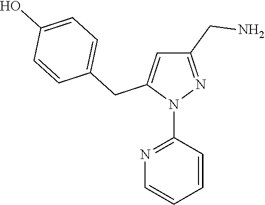 [SMILES: NCc1cc(Cc2ccc(O)cc2)n(-c2ccccn2)n1] | 4-{[3- (Aminomethyl)-1- (pyridin-2-yl)-1H- pyrazol-5- yl]methyl}phenol | F | 12.64 | 281 |
| 44 | 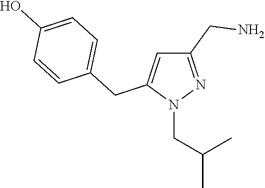 [SMILES: CC(C)Cn1nc(CN)cc1Cc1ccc(O)cc1] | 4-{[3- (Aminomethyl)-1- isobutyl-1H- pyrazol-5- yl]methyl}phenol | F | 13.75 | 260 |
| 45 | 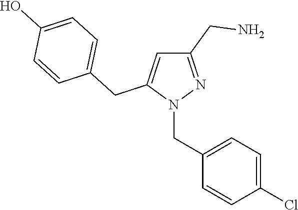 [SMILES: NCc1cc(Cc2ccc(O)cc2)n(Cc2ccc(Cl)cc2)n1] | 4-{[3- (Aminomethyl)-1- (4-chlorobenzyl)- 1H-pyrazol-5- yl]methyl}phenol | F | 14.90 | 328 |
| 46 | 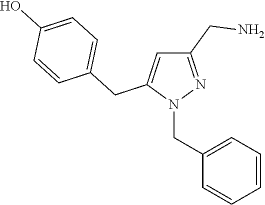 [SMILES: NCc1cc(Cc2ccc(O)cc2)n(Cc2ccccc2)n1] | 4-{[3- (Aminomethyl)-1- benzyl-1H- pyrazol-5- yl]methyl}phenol | F | 13.76 | 294 |
| 47 | 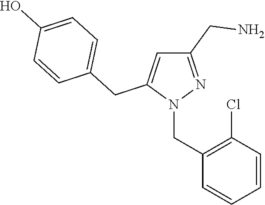 [SMILES: NCc1cc(Cc2ccc(O)cc2)n(Cc2ccccc2Cl)n1] | 4-{[3- (Aminomethyl)-1- (2-chlorobenzyl)- 1H-pyrazol-5- yl]methyl}phenol | F | 14.80 | 328 |
| 48 | 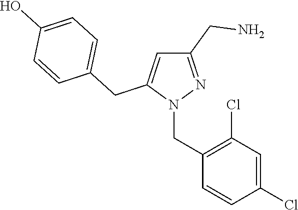 [SMILES: NCc1cc(Cc2ccc(O)cc2)n(Cc2ccc(Cl)cc2Cl)n1] | 4-{[3- (Aminomethyl)-1- (2-chlorobenzyl)- 1H-pyrazol-5- yl]methyl}phenol | F | 15.82 | 362 |
| 49 | 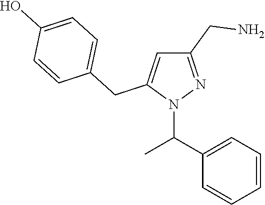 [SMILES: CC(c1ccccc1)n1nc(CN)cc1Cc1ccc(O)cc1] | 4-{[3- (Aminomethyl)-1- (1-phenylethyl)- 1H-pyrazol-5- yl]methyl}phenol | F | 14.65 | 308 |
| 50 | 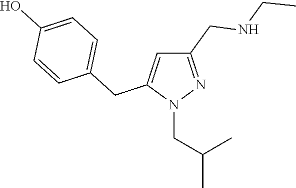 [SMILES: CCNCc1cc(Cc2ccc(O)cc2)n(CC(C)C)n1] | 4-({3- [{Ethylamino) methyl]-1-isobutyl- 1H-pyrazol-5- yl}methyl)phenol | F | 14.31 | 288 |
| 51 | 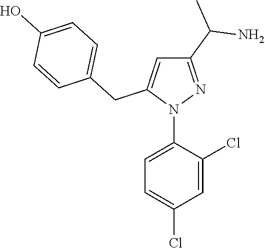 [SMILES: CC(N)c1cc(Cc2ccc(O)cc2)n(-c2ccc(Cl)cc2Cl)n1] | 4-{[3-(1- Aminoethyl)-1- (2,4- dichlorophenyl)- 1H-pyrazol-5- yl]methyl}phenol | F | 16.31 | 362 |
| 52 | 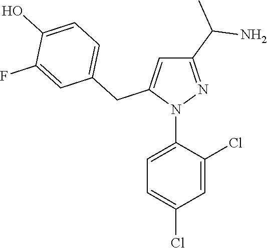 [SMILES: CC(N)c1cc(Cc2ccc(O)c(F)c2)n(-c2ccc(Cl)cc2Cl)n1] | 4-{[3-(1- Aminoethyl)-1- (2,4- dichlorophenyl)- 1H-pyrazol-5- yl]methyl}-2- fluorophenol | F | 16.35 | 380 |
| 53 | 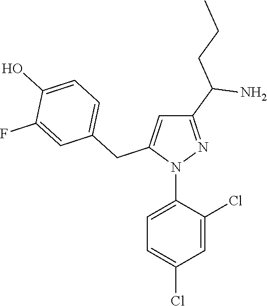 [SMILES: CCCC(N)c1cc(Cc2ccc(O)c(F)c2)n(-c2ccc(Cl)cc2Cl)n1] | 4-{[3-(1- Aminobutyl)-1- (2,4- dichlorophenyl)- 1H-pyrazol-5- yl]methyl}-2- fluorophenol | F | 17.19 | 408 |
| 54 | 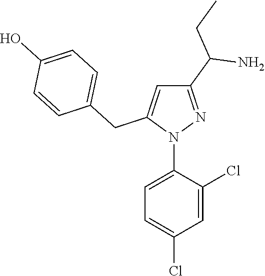 [SMILES: CCC(N)c1cc(Cc2ccc(O)cc2)n(-c2ccc(Cl)cc2Cl)n1] | 4-{[3-(1- Aminopropyl)-1- (2,4- dichlorophenyl)- 1H-pyrazol-5- yl]methyl}phenol | F | 16.60 | 376 |
| 55 | 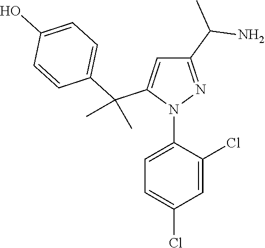 [SMILES: CC(N)c1cc(C(C)(C)c2ccc(O)cc2)n(-c2ccc(Cl)cc2Cl)n1] | 4-{2-[3-(1- Aminoethyl)-1- (2,4- dichlorophenyl)- 1H-pyrazol-5- yl]propan-2- yl]phenol | F | 17.00 | 390 |

### Example 56. 4-{[3-(2-Aminopropan-2-yl)-1-(2,4-dichlorophenyl)-1H-pyrazol-5-yl]methyl}-2-fluorophenol

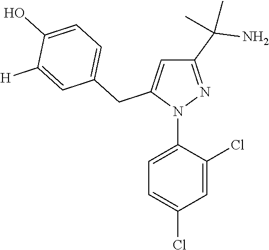 [SMILES: CC(C)(N)c1cc(Cc2ccc(O)cc2)n(-c2ccc(Cl)cc2Cl)n1]

### a) 2-[1-(2,4-Dichlorophenyl)-5-(3-fluoro-4-methoxybenzyl)-1H-pyrazol-3-yl]propan-2-ol

MeMgBr (3.0 M solution in Et₂O, 2.54 mL, 7.62 mmol) was added to a 0° C. cooled solution of methyl 1-(2,4-dichlorophenyl)-5-(3-fluoro-4-methoxybenzyl)-1H-pyrazole-3-carboxylate (Intermediate D13, 1.04 g, 2.54 mmol) in THF (12 mL) and the reaction was stirred at rt for 24 h. The mixture was poured into water (50 mL) and extracted with EtOAc (2×50 mL). The combined organic layers were dried over Na₂SO₄ (anhydrous), filtered and concentrated. The crude residue was purified by flash chromatography on SiO₂ (30% EtOAc/hexanes) to afford 377 mg the title compound (yellow foam, 377 mg, 77% yield).

HPLC-MS (Method D): Ret, 10.78 min; ESI⁺-MS m/z: 409 (M+1).

### b) 3-(2-Azidopropan-2-yl)-1-(2,4-dichlorophenyl)-5-(3-fluoro-4-methoxybenzyl)-1H-pyrazole

TFA (1.05 mL, 13.68 mmol) was added dropwise to a mixture of NaN₃ (0.38 g, 5.86 mmol) and the previous compound (0.80 g, 1.95 mmol) in CH₂Cl₂ (20 mL) cooled at 0° C. The reaction mixture was allowed to reach rt and was stirred for 2 d. The resulting suspension was diluted with CH₂Cl₂ (30 mL) and was washed with NH₄Cl (saturated aqueous solution, 20 mL). The organic layer was dried over Na₂SO₄ (anhydrous), filtered and concentrated, rendering the title compound (yellow oil, 0.76 g, 89% yield), which was submitted to the next step without further purification.

HPLC-MS (Method D): Ret, 11.70 min; ESI⁺-MS m/z: 434 (M+1).

### c) 2-[1-(2,4-Dichlorophenyl)-5-(3-fluoro-4-methoxybenzyl)-1H-pyrazol-3-yl]propan-2-amine

SnC₂.2H₂O (0.78 g, 3.45 mmol) was added to a solution of the previous compound (0.75 g, 1.73 mmol) in EtOH (10 mL) and the mixture was stirred at rt for 24 h. The solvent was removed by rotatory evaporation in the presence of SiO₂ and the residue was purified by flash chromatography on SiO₂ (60% EtOAc/hexanes) to give the title compound (colorless oil, 0.42 g, 59% yield).

HPLC-MS (Method D): Ret, 9.91 min; ESI⁺-MS m/z: 408 (M+1).

### d) Title Compound

A solution of the previous compound (0.40 g, 0.98 mmol) in HBr (48% aqueous solution, 4 mL) was heated at 100° C. for 6 h. The mixture was allowed to reach rt and the solvent was concentrated off. The crude residue was suspended in a small amount of CH₂Cl₂/MeOH/NH₄OH (95:5:1) to charge it in the column. Purification by flash chromatography on SiO₂ (CH₂Cl₂/MeOH/NH₄OH 95:5:1→90:8:1) afforded the title compound (white solid, 0.27 g, 69% yield)

HPLC-MS (Method F): Ret, 16.23 min; ESI⁺-MS m/z: 394 (M+1).

This method was used for the preparation of example 57 using suitable starting materials, reduction of the azide function was carried out by hydrogenation over Pt—C catalyst:

| | | | | Ret | MS |
|---|---|---|---|---|---|
| Ex | Structure | Chemical name | Method | (min) | (M + H) |
| 57 | 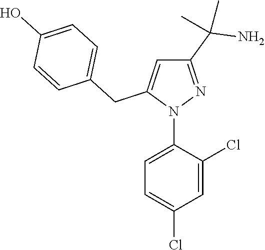 [SMILES: CC(C)(N)c1cc(Cc2ccc(O)cc2)n(-c2ccc(Cl)cc2Cl)n1] | 4-{[3-(2- Aminopropan-2- yl)-1-(2,4- dichlorophenyl)- 1H-pyrazol-5- yl]methyl}phenol | F | 16.97 | 376 |

### Example 58. 4-{[3-(2-Aminoethyl)-1-(2,4-dichlorophenyl)-1H-pyrazol-5-yl]methyl}phenol

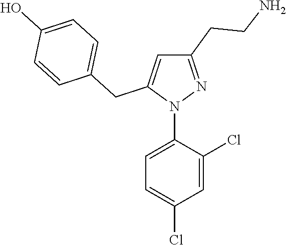 [SMILES: NCCc1cc(Cc2ccc(O)cc2)n(-c2ccc(Cl)cc2Cl)n1]

### a) 2-[1-(2,4-Dichlorophenyl)-5-(4-methoxybenzyl)-1H-pyrazol-3-yl]acetonitrile

Methanesulfonyl chloride (0.20 mL, 2.52 mmol) was added to a 0° C. cooled solution of [1-(2,4-dichlorophenyl)-5-(4-methoxybenzyl)-1H-pyrazol-3-yl]methanol (Intermediate E1, step a, 0.83 g, 2.29 mmol) and Et₃N (0.48 mL, 3.44 mmol) in CH₂Cl₂ (10 mL). The reaction mixture was stirred for 1 h, poured into water (10 mL) and extracted with CH₂Cl₂ (2×10 mL). The combined organic layers were dried over Na₂SO₄ (anhydrous), filtered and concentrated, to afford 1.01 g of the corresponding methanesulfonate (pale yellow oil). This oil was dissolved in DMF (10 mL) and stirred at rt for 2.5 h in the presence of NaCN (0.28 g, 5.72 mmol). The mixture was poured into water (40 mL) and extracted with EtOAc (30 mL). The organic layer was washed with water (20 mL), dried over Na₂SO₄ (anhydrous), filtered and concentrated, rendering the title compound (brown oil, 0.78 g, 92% yield).

HPLC-MS (Method D): Ret, 10.87 min; ESI⁺-MS m/z: 372 (M+1).

### b) 2-[1-(2,4-Dichlorophenyl)-5-(4-methoxybenzyl)-1H-pyrazol-3-yl]ethanamine

Borane (1 M solution in THF, 4.11 mL, 4.11 mmol) was added to a solution of the previous compound (0.77 g, 2.06 mmol) in THF (10 mL) and the reaction mixture was heated under reflux for 8 h. After cooling down to rt, NaOH (10% aqueous solution, 1 mL) was added and the mixture was heated under reflux for 30 min. After this time, the mixture was cooled down to rt and volatiles were removed by rotatory evaporation. The residue was dissolved in EtOAc (40 mL) and was washed with NaOH (5% aqueous solution, 20 mL). The organic layer was dried over Na₂SO₄ (anhydrous), filtered and concentrated. The residue was purified by flash chromatography on SiO₂ (CH₂Cl₂/MeOH/NH₄OH 95:5:1→90:10:1) to afford the title compound (pale yellow oil, 0.26 g, 34% yield).

HPLC-MS (Method D): Ret, 9.73 min; ESI⁺-MS m/z: 376 (M+1).

### c) Title Compound

A solution of the previous compound (0.14 g, 0.38 mmol) in HBr (48% aqueous solution, 3 mL) was heated under reflux for 2.5 h. The mixture was allowed to reach rt and the solvent was concentrated off. The crude residue was suspended in a small amount of CH₂Cl₂/MeOH/NH₄OH (95:5:1) to charge it in the column. Purification by flash chromatography on SiO₂ (CH₂Cl₂/MeOH/NH₄OH 95:5:1→90:10:1) afforded the title compound (pale yellow solid, 0.11 g, 77% yield)

HPLC-MS (Method F): Ret, 15.51 min; ESI⁺-MS m/z: 362 (M+1).

### Example 59. 4-{[3-(1-Aminopropan-2-yl)-1-(2,4-dichlorophenyl)-1H-pyrazol-5-yl]methyl}phenol

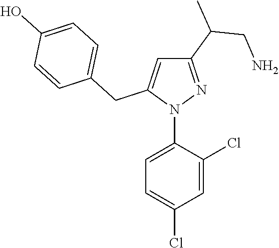 [SMILES: CC(CN)c1cc(Cc2ccc(O)cc2)n(-c2ccc(Cl)cc2Cl)n1]

### a) 1-[1-(2,4-Dichlorophenyl)-5-(4-methoxybenzyl)-1H-pyrazol-3-yl]ethanol

MeMgBr (3.0 M solution in Et₂O, 0.80 mL, 2.40 mmol) was added to a 0° C. cooled solution of 1-(2,4-dichlorophenyl)-5-(4-methoxybenzyl)-1H-pyrazole-3-carbaldehyde (Intermediate E1, 0.72 g, 2.00 mmol) in THF (10 mL) and the reaction was stirred for 19 h. The mixture was poured into NH₄Cl (saturated aqueous solution, 20 mL) and extracted with EtOAc (2×30 mL). The combined organic layers were dried over Na₂SO₄ (anhydrous), filtered and concentrated. The crude residue was purified by flash chromatography on SiO₂ (20% EtOAc/hexanes) to afford the title compound (yellow oil, 0.65 g, 87% yield).

HPLC-MS (Method B): Ret, 10.80 min; ESI⁺-MS m/z: 377 (M+1).

### b) 1-[1-(2,4-Dichlorophenyl)-5-(4-methoxybenzyl)-1H-pyrazol-3-yl]ethanone

MnO₂ (88% purity, 1.45 g, 16.68 mmol) was added to a solution of the previous compound (0.63 g, 1.67 mmol) in CH₂Cl₂ (10 mL) and the mixture was heated under reflux for 2 h. The reaction mixture was allowed to cool down to rt, filtered through a pad of Celite, rinsed with CH₂Cl₂ (30 mL) and the solvent was concentrated off, affording the title compound (yellow foam, 0.59 g, 94% yield), which was submitted to the next step without further purification.

HPLC-MS (Method B): Ret, 11.35 min; ESI⁺-MS m/z: 375 (M+1).

### c) 2-[1-(2,4-Dichlorophenyl)-5-(4-methoxybenzyl)-1H-pyrazol-3-yl]propanenitrile

A solution of t-BuOK (0.23 g, 2.03 mmol) in t-BuOH (5 mL) was added dropwise to a −15° C. cooled solution of TOSMIC (0.20 g, 1.01 mmol) and 1-[1-(2,4-dichlorophenyl)-5-(4-methoxybenzyl)-1H-pyrazol-3-yl]ethanone (0.38 g, 1.01 mmol) in DME (10 mL). After 1 h the reaction mixture was allowed to reach rt and stirred for additional 3 h. The reaction volume was reduced to ⅓ by rotatory evaporation and the residue was dissolved in EtOAc (30 mL) and was washed with water (2×30 mL). The organic layer was dried over Na₂SO₄ (anhydrous), filtered and concentrated, to give the title compound (yellow foam, 0.39 g) which was used without further purification.

HPLC-MS (Method B): Ret, 11.23 min; ESI⁺-MS m/z: 386 (M+1).

### d) Title Compound

The title compound was obtained following the procedure described in Example 58, steps a and b, and using 2-[1-(2,4-dichlorophenyl)-5-(4-methoxybenzyl)-1H-pyrazol-3-yl]propanenitrile as starting material.

HPLC-MS (Method F): Ret, 16.43 min; ESI⁺-MS m/z: 376 (M+1).

This method was used for the preparation of example 60 using suitable starting materials:

| | | | | Ret | MS |
|---|---|---|---|---|---|
| Ex | Structure | Chemical name | Method | (min) | (M + H) |
| 60 | 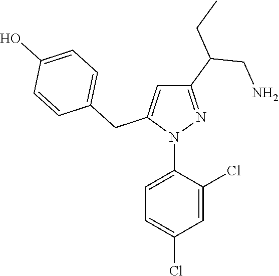 [SMILES: CCC(CN)c1cc(Cc2ccc(O)cc2)n(-c2ccc(Cl)cc2Cl)n1] | 4-{[3-(1- Aminobutan-2-yl)- 1-(2,4- dichlorophenyl)- 1H-pyrazol-5- yl]methyl}phenol | F | 17.15 | 390 |

### Examples 61 and 62. (S)-4-{[3-(1-Aminoethyl)-1-(2,4-dichlorophenyl)-1H-pyrazol-5-yl]methyl}phenol and (R)-4-{[3-(1-Aminoethyl)-1-(2,4-dichlorophenyl)-1H-pyrazol-5-yl]methyl}phenol

Examples 61 and 62 were obtained by chiral preparative HPLC from example 51. Column: Chiralpak IA; Temperature: ambient; Flow: 10 mL/min; Mobile phase: n-Heptane/(IPA+0.33% DEA) 70/30 v/v.

Example 61 HPLC-MS (Method F): Ret, 16.31 min; ESI⁺-MS m/z, 362 (M+1).

Example 62 HPLC-MS (Method F): Ret, 16.31 min; ESI⁺-MS m/z, 362 (M+1).

### Examples 63 and 64. (S)-4-{[3-(1-Aminoethyl)-1-(2,4-dichlorophenyl)-1H-pyrazol-5-yl]methyl}-2-fluorophenol and (R)-4-{[3-(1-Aminoethyl)-1-(2,4-dichlorophenyl)-1H-pyrazol-5-yl]methyl}-2-fluorophenol

Examples 63 and 64 were obtained by chiral preparative HPLC from example 52. Column: Chiralpak IA; Temperature: ambient; Flow: 10 mL/min; Mobile phase: n-Heptane/(IPA+0.33% DEA) 70/30 v/v.

Example 61 HPLC-MS (Method F): Ret, 16.35 min; ESI⁺-MS m/z, 380 (M+1).

Example 62 HPLC-MS (Method F): Ret, 16.35 min; ESI⁺-MS m/z, 380 (M+1).

### Example 65. 4-{[3-(1-Aminoethyl)-1-cyclohexyl-1H-pyrazol-5-yl]methyl}phenol

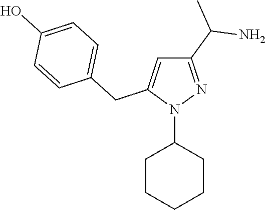 [SMILES: CC(N)c1cc(Cc2ccc(O)cc2)n(C2CCCCC2)n1]

### a) Ethyl 1-cyclohexyl-5-(4-methoxybenzyl)-1H-pyrazole-3-carboxylate

The title compound was obtained following the procedure described in Intermediate D1 and using ethyl 5-(4-methoxyphenyl)-2,4-dioxopentanoate and cyclohexylhydrazine hydrochloride as starting materials.

HPLC-MS (Method B): Ret, 11.30 min; ESI⁺-MS m/z: 343 (M+1).

### b) 1-Cyclohexyl-5-(4-methoxybenzyl)-1H-pyrazole-3-carbaldehyde

The title compound was obtained following the procedure described in Intermediate E1 and using the compound obtained in step a as starting material.

HPLC-MS (Method B): Ret, 11.03 min; ESI⁺-MS m/z: 299 (M+1).

### c) 1-[1-Cyclohexyl-5-(4-methoxybenzyl)-1H-pyrazol-3-yl]ethan-1-amine

The title compound was obtained following the procedure described in Example 24 and using the compound obtained in step b as starting material.

HPLC-MS (Method B): Ret, 9.89 min; ESI⁺-MS m/z: 314 (M+1).

### d) Title Compound

The title compound was obtained following the procedure described in Example 38 and using the compound obtained in step c as starting material.

HPLC-MS (Method F): Ret, 14.83 min; ESI⁺-MS m/z: 300 (M+1).

### Example 66. 4-{[3-(1-Aminoethyl)-1-(tetrahydro-2H-pyran-4-yl)-1H-pyrazol-5-yl]methyl}phenol

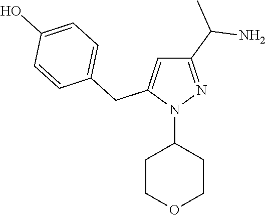 [SMILES: CC(N)c1cc(Cc2ccc(O)cc2)n(C2CCOCC2)n1]

The title compound was obtained following the procedure described in Example 65, and using ethyl 5-(4-methoxyphenyl)-2,4-dioxopentanoate and (tetrahydro-2H-pyran-4-yl)hydrazine as starting materials.

HPLC-MS (Method F): Ret, 12.18 min; ESI⁺-MS m/z: 302 (M+1).

### Example 67. 4-{[3-(1-Aminoethyl)-1-phenethyl-1H-pyrazol-5-yl]methyl}phenol

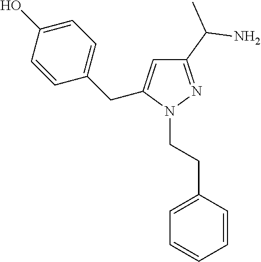 [SMILES: CC(N)c1cc(Cc2ccc(O)cc2)n(CCc2ccccc2)n1]

### a) Methyl 5-(4-methoxybenzyl)-1-phenethyl-1H-pyrazole-3-carboxylate

The title compound was obtained following the procedure described in Intermediate D25 and using methyl 5-(4-methoxybenzyl)-1H-pyrazole-3-carboxylate and (2-bromoethyl)benzene as starting materials.

HPLC-MS (Method B): Ret, 10.90 min; ESI⁺-MS m/z: 351 (M+1).

### b) Title Compound

The title compound was obtained following the procedure described in Example 65 steps b-d and using the compound obtained in step a as starting material.

HPLC-MS (Method F): Ret, 14.84 min; ESI⁺-MS m/z: 322 (M+1).

### Example 68. 4-{[3-(2-Aminopropyl)-1-(2,4-dichlorophenyl)-1H-pyrazol-5-yl]methyl}phenol

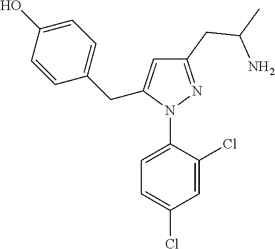 [SMILES: CC(N)Cc1cc(Cc2ccc(O)cc2)n(-c2ccc(Cl)cc2Cl)n1]

### a) 1-(2,4-Dichlorophenyl)-5-(4-methoxybenzyl)-3-(2-nitropropyl)-1H-pyrazole

A mixture of 1-(2,4-dichlorophenyl)-5-(4-methoxybenzyl)-1H-pyrazole-3-carbaldehyde (Intermediate E1, 0.42 g, 1.16 mmol), nitroethane (0.67 mL, 9.28 mmol) and NH₄OAc (0.03 g, 0.35 mmol) was heated at 120° C. for 16 h. The mixture was cooled down to rt, diluted with CH₂Cl₂ (30 mL) and washed with water (2×20 mL); the organic layer was dried over Na₂SO₄ (anhydrous), filtered and concentrated, rendering 0.48 g of an orange oil. This oil was dissolved in CHCl₃ (12 mL) and i-PrOH (4 mL); SiO₂ (1.15 g) was added, followed by NaBH₄ (0.09 g, 2.30 mmol, added in 3 portions) and stirred at rt for 20 h. More NaBH₄ (0.09 g, 2.30 mmol) was added. After 6 h volatiles were removed by rotatory evaporation in the presence of SiO₂ and the residue was purified by flash chromatography on SiO₂ (12→21% EtOAc/hexanes) to afford the title compound (orange oil, 0.36 g, 74% yield).

HPLC-MS (Method B): Ret, 11.39 min; ESI⁺-MS m/z: 420 (M+1).

### b) 4-{[3-(2-Aminopropyl)-1-(2,4-dichlorophenyl)-1H-pyrazol-5-yl]methyl}phenol

Fe (0.20 g, 3.52 mmol) was added to a solution of the compound obtained in step a (148 mg, 0.35 mmol) and NH₄Cl (75 mg, 1.40 mmol) in water (4 mL) and MeOH (12 mL). The reaction mixture was warmed up to 50° C. and stirred at this temperature for 1.5 h. The mixture was cooled down to rt, filtered and rinsed with EtOAc (30 mL). The filtrate was diluted with EtOAc (50 mL) and washed with water (60 mL); the organic layer was dried over Na₂SO₄ (anhydrous), filtered and concentrated, rendering 122 mg of a colorless oil. This oil was submitted to the procedure described in Example 38, affording the title compound.

HPLC-MS (Method H): Ret, 16.05 min; ESI⁺-MS m/z: 376 (M+1).

### Example 69. (R)-4-{[3-(1-Aminoethyl)-1-(2-chloro-4-methoxyphenyl)-1H-pyrazol-5-yl]methyl}phenol

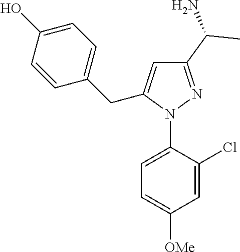 [SMILES: COc1ccc(-n2nc([C@@H](C)N)cc2Cc2ccc(O)cc2)c(Cl)c1]

### a) 1-[1-(2-Chloro-4-methoxyphenyl)-5-{4-[(tetrahydro-2H-pyran-2-yl)oxy]benzyl}-1H-pyrazol-3-yl]ethan-1-one

The title compound was obtained following the procedure described in Example 59 and using intermediate E16 as starting material.

HPLC-MS (Method B): Ret, 11.49 min; ESI⁺-MS m/z: 441 (M+1).

### b) (S)—N—{(E)-1-[1-(2-chloro-4-methoxyphenyl)-5-{4-[(tetrahydro-2H-pyran-2-yl)oxy]benzyl}-1H-pyrazol-3-yl]ethylidene}-2-methylpropane-2-sulfinamide

A suspension of the compound obtained in step a (264 mg, 0.60 mmol), (S)-2-methylpropane-2-sulfinamide (90 mg, 0.74 mmol) and Ti(OEt)₄/TiO₂ (0.29 mL, 1.40 mmol) was heated at 72° C. for 15 h under Ar atmosphere. The reaction mixture was allowed to reach rt and was diluted with EtOAc (8 mL) and brine (1 mL). The white suspension was stirred for 30 min, filtered through a pad of Celite, rinsed with EtOAc (30 mL) and the solvent was concentrated. The crude residue was purified by flash chromatography on SiO₂ (31→40% EtOAc/hexanes) to afford 238 mg of the title compound (yellow foam, 65% yield).

HPLC-MS (Method B): Ret, 11.92 min; ESI⁺-MS m/z: 544 (M+1).

### c) (S)—N-{(1R)-1-[1-(2-chloro-4-methoxyphenyl)-5-{4-[(tetrahydro-2H-pyran-2-yl)oxy]benzyl}-1H-pyrazol-3-yl]ethyl}-2-methylpropane-2-sulfinamide

L-Selectride (1.0 M solution in THF, 1.68 mL, 1.68 mmol) was added dropwise (20 min) to a −48° C. cooled solution of (the compound obtained in step b (228 mg, 0.42 mmol) in THF (10 mL) and stirred for 30 min at that temperature and for 15 h at rt. The reaction mixture was cooled to 0° C. and MeOH (2 mL) was added dropwise; after 1 h the mixture was diluted with EtOAc (20 mL) and washed with brine (2×10 mL), the combined aqueous layers being extracted with EtOAc (30 mL). The combined organic layers were dried over Na₂SO₄ (anhydrous), filtered and concentrated, rendering 445 mg the title compound (yellow oil, >100% yield, crude) which was submitted to the next step without further purification.

HPLC-MS (Method B): Ret, 11.60 min; ESI⁺-MS m/z: 546 (M+1).

### d) Title Compound

The title compound was obtained following the procedure described in Example 24 step c and using the compound obtained in step c as starting material.

HPLC-MS (Method F): Ret, 14.83 min; ESI⁺-MS m/z: 358 (M+1).

### Example 70. (S)-4-{[3-(1-Aminoethyl)-1-(2-chloro-4-methoxyphenyl)-1H-pyrazol-5-yl]methyl}phenol

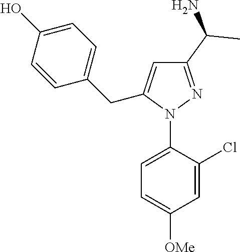 [SMILES: COc1ccc(-n2nc([C@H](C)N)cc2Cc2ccc(O)cc2)c(Cl)c1]

The title compound was obtained following the procedure described in Example 69 and using (R)-2-methylpropane-2-sulfinamide as starting material.

HPLC-MS (Method F): Ret, 14.83 min; ESI⁺-MS m/z: 358 (M+1).

### Example 71. 4-{[3-(1-Amino-2-hydroxyethyl)-1-(2,4-dichlorophenyl)-1H-pyrazol-5-yl]methyl}phenol

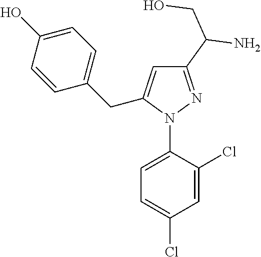 [SMILES: NC(CO)c1cc(Cc2ccc(O)cc2)n(-c2ccc(Cl)cc2Cl)n1]

### a) 2-Amino-2-[1-(2,4-dichlorophenyl)-5-(4-methoxybenzyl)-1H-pyrazol-3-yl]ethan-1-ol

The title compound was obtained following the procedure described in J. Org. Chem. 2000, 65, 2856-2862 and using N-{[1-(2,4-dichlorophenyl)-5-(4-methoxybenzyl)-1H-pyrazol-3-yl]methylene}-2-methylpropane-2-sulfinamide (Example 24, step a) as starting material.

HPLC-MS (Method B): Ret, 9.93 min; ESI⁺-MS m/z: 392 (M+1).

### b) Title Compound

The title compound was obtained following the procedure described in Example 38 and using the compound obtained in step a as starting material.

HPLC-MS (Method F): Ret, 15.30 min; ESI⁺-MS m/z: 378 (M+1).

### Example 72. 4-({3-[1-Amino-2-(piperidin-1-yl)ethyl]-1-(2,4-dichlorophenyl)-1H-pyrazol-5-yl}methyl)phenol

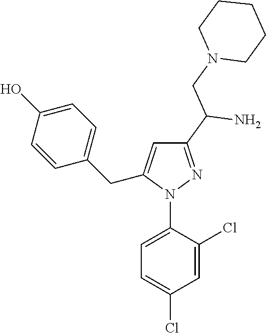 [SMILES: NC(CN1CCCCC1)c1cc(Cc2ccc(O)cc2)n(-c2ccc(Cl)cc2Cl)n1]

### a) 2-Bromo-1-[1-(2,4-dichlorophenyl)-5-(4-methoxybenzyl)-1H-pyrazol-3-yl]ethan-1-one

CuBr₂ (1.30 g, 5.86 mmol) was added to a solution of 1-[1-(2,4-dichlorophenyl)-5-(4-methoxybenzyl)-1H-pyrazol-3-yl]ethanone (Example 59, step b; 1.10 g, 2.93 mmol) in EtOAc (7.5 mL) and CHCl₃ (7.5 mL) and the mixture was heated at 85° C. for 80 min. The reaction mixture was allowed to reach rt, filtered through a pad of Celite, rinsed with CH₂Cl₂ (50 mL) and the solvent was concentrated off. The crude residue was purified by flash chromatography on SiO₂ (15% EtOAc/hexanes) to afford the title compound (yellow oil, 1.30 g, 98% yield).

HPLC-MS (Method B): Ret, 11.53 min; ESI⁺-MS m/z: 455 (M+1).

### b) 1-[1-(2,4-Dichlorophenyl)-5-(4-methoxybenzyl)-1H-pyrazol-3-yl]-2-(piperidin-1-yl)ethan-1-one

A mixture of K₂CO₃ (0.26 g, 1.85 mmol), piperidine (0.17 mL, 1.68 mmol) and the compound obtained in step a (0.38 g, 0.84 mmol) in CH₃CN (10 mL) was stirred at rt for 4 h. The reaction mixture was diluted with EtOAc (25 mL) and water (25 mL); the aqueous layer was extracted with EtOAc (2×15 mL) and the combined organic layers were dried over Na₂SO₄ (anhydrous), filtered and concentrated. The crude residue was purified by flash chromatography on SiO₂ (0→17% MeOH/CH₂Cl₂) to afford 0.33 g of the title compound (yellow foam, 84% yield).

HPLC-MS (Method B): Ret, 11.60 min; ESI⁺-MS m/z: 458 (M+1).

### c) 1-[1-(2,4-Dichlorophenyl)-5-(4-methoxybenzyl)-1H-pyrazol-3-yl]-2-(piperidin-1-yl)ethan-1-ol

NaBH₄ (0.11 g, 1.43 mmol) was added in portions to a 0° C. cooled solution of the compound obtained in step b (0.66 g, 1.43 mmol) in MeOH (15 mL). After 3.5 h the mixture was poured over NH₄Cl (saturated aqueous solution, 20 mL) and was extracted with EtOAc (4×20 mL); the combined organic layers were dried over Na₂SO₄ (anhydrous), filtered and concentrated. The crude residue was purified by flash chromatography on SiO₂ (3→17% MeOH/CH₂Cl₂) to afford the title compound (yellow oil, 0.46 g, 69% yield).

HPLC-MS (Method B): Ret, 10.41 min; ESI⁺-MS m/z: 460 (M+1).

### d) 1-{2-Azido-2-[1-(2,4-dichlorophenyl)-5-(4-methoxybenzyl)-1H-pyrazol-3-yl]ethyl}piperidine

Methanesulfonyl chloride (0.09 mL, 1.15 mmol) was added to a 0° C. cooled solution of the compound obtained in step c (0.44 g, 0.96 mmol) and Et₃N (0.20 mL, 1.44 mmol) in CH₂Cl₂ (15 mL). The reaction mixture was stirred at rt for 3 h, poured into brine (10 mL) and extracted with CH₂Cl₂ (2×15 mL). The combined organic layers were dried over Na₂SO₄ (anhydrous), filtered and concentrated, to afford 0.58 g of the corresponding methanesulfonate (yellow solid). This oil was dissolved in DMF (8 mL) and stirred at rt for 17 h in the presence of NaN₃ (0.12 g, 1.92 mmol). The mixture was poured into NaHCO₃ (saturated aqueous solution, 15 mL) and extracted with EtOAc (3×20 mL). The combined organic layers were dried over Na₂SO₄ (anhydrous), filtered and concentrated. The residue was purified by flash chromatography on SiO₂ (CH₂Cl₂/MeOH/NH₃ 100:0:0→95:5:1) to afford the title compound (yellow oil, 0.25 g, 52% yield).

HPLC-MS (Method B): Ret, 12.00 min; ESI⁺-MS m/z: 485 (M+1).

### e) 1-[1-(2,4-Dichlorophenyl)-5-(4-methoxybenzyl)-1H-pyrazol-3-yl]-2-(piperidin-1-yl)ethan-1-amine

PPh₃ (0.19 g, 0.73 mmol) and water (0.18 mL, 10 mmol) were added to a solution of the compound obtained in step d (0.24 g, 0.49 mmol) in THF (8 mL). The reaction mixture was stirred at rt for 18 h, poured into brine (10 mL) and extracted with EtOAc (3×20 mL). The combined organic layers were dried over Na₂SO₄ (anhydrous), filtered and concentrated. The residue was purified by flash chromatography on SiO₂ (CH₂Cl₂/MeOH/NH₃ 95:5:1→86:14:1) to afford the title compound (yellow oil, 0.19 g, 83% yield).

HPLC-MS (Method B): Ret, 10.65 min; ESI⁺-MS m/z: 459 (M+1).

### f) 4-({3-[1-Amino-2-(piperidin-1-yl)ethyl]-1-(2,4-dichlorophenyl)-1H-pyrazol-5-yl}methyl)phenol

The title compound was obtained following the procedure described in Example 38 and using the compound obtained in step e as starting material.

HPLC-MS (Method I): Ret, 16.86 min; ESI⁺-MS m/z: 445 (M+1).

### Example 73. 4-{[3-(1-Amino-2-morpholinoethyl)-1-(2,4-dichlorophenyl)-1H-pyrazol-5-yl]methyl}phenol

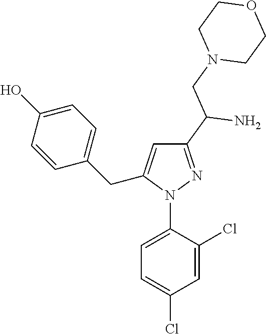 [SMILES: NC(CN1CCOCC1)c1cc(Cc2ccc(O)cc2)n(-c2ccc(Cl)cc2Cl)n1]

The title compound was obtained following the procedure described in Example 72 and using morpholine as starting material in step b.

HPLC-MS (Method I): Ret, 16.33 min; ESI⁺-MS m/z: 447 (M+1).

### Example 74. N-(4-{[3-(Aminomethyl)-1-(2,4-dichlorophenyl)-1H-pyrazol-5-yl]methyl}phenyl)thiazol-2-amine

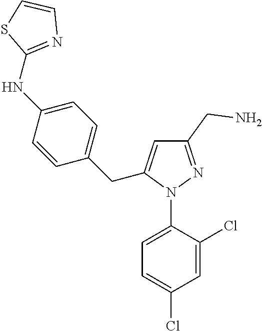 [SMILES: NCc1cc(Cc2ccc(Nc3nccs3)cc2)n(-c2ccc(Cl)cc2Cl)n1]

### a) Methyl 1-(2,4-dichlorophenyl)-5-[4-(thiazol-2-ylamino)benzyl]-1H-pyrazole-3-carboxylate

A mixture of 2-bromothiazole (1.62 g, 9.87 mmol), pTsOH (0.25 g, 1.33 mmol) and methyl 5-(4-aminobenzyl)-1-(2,4-dichlorophenyl)-1H-pyrazole-3-carboxylate (intermediate D32, step b; 1.00 g, 2.66 mmol) was heated under reflux for 24 h. More 2-bromothiazole (0.44 g, 2.66 mmol) and pTsOH (0.25 g, 1.33 mmol) were added and stirring continued for 12 h. The mixture was cooled down to rt, diluted with EtOAc (50 mL) and washed with NaHCO₃ (saturated aqueous solution, 30 mL); the organic layer was dried over Na₂SO₄ (anhydrous), filtered and concentrated. The residue was purified by flash chromatography on SiO₂ (15→30% EtOAc/hexanes) to afford the title compound contaminated with iso-propyl ester (pale yellow solid, 1.12 g, 92% yield).

HPLC-MS (Method B): Ret, 10.93 min; ESI⁺-MS m/z: 459 (M+1).

### b) N-(4-{[3-(Aminomethyl)-1-(2,4-dichlorophenyl)-1H-pyrazol-5-yl]methyl}phenyl)thiazol-2-amine

The title compound was obtained following the procedure described in Example 72 and using the compound obtained in step a, as starting material.

HPLC-MS (Method I): Ret, 16.50 min; ESI⁺-MS m/z: 430 (M+1).

### Example 75. 2-(4-{[3-(1-Aminoethyl)-1-(2,4-dichlorophenyl)-1H-pyrazol-5-yl]methyl}phenoxy)ethan-1-ol

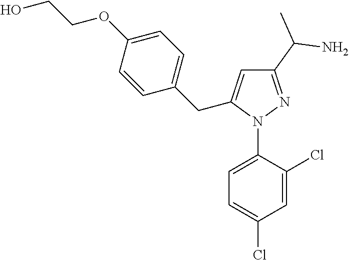 [SMILES: CC(N)c1cc(Cc2ccc(OCCO)cc2)n(-c2ccc(Cl)cc2Cl)n1]

### a) tert-Butyl {1-[1-(2,4-dichlorophenyl)-5-(4-hydroxybenzyl)-1H-pyrazol-3-yl]ethyl}carbamate

Boc₂O (0.60 g, 2.73 mmol) and Et₃N (0.42 mL, 3.00 mmol) were added to a solution of 4-{[3-(1-aminoethyl)-1-(2,4-dichlorophenyl)-1H-pyrazol-5-yl]methyl}phenol (Example 51; 0.99 g, 2.73 mmol) in CH₂Cl₂ (15 mL) and the mixture was stirred at rt for 16 h. The reaction mixture was diluted with CH₂Cl₂ (25 mL) and NH₄Cl (saturated aqueous solution, 20 mL); the organic layer was dried over Na₂SO₄ (anhydrous), filtered and concentrated. The crude residue was purified by flash chromatography on SiO₂ (CH₂Cl₂/MeOH/NH₃ 97:3:1) to afford 0.59 g of the title compound (white foam, 47% yield).

HPLC-MS (Method B): Ret, 11.02 min; ESI⁺-MS m/z: 462 (M+1).

### b) tert-Butyl {1-[1-(2,4-dichlorophenyl)-5-(4-{2-[(tetrahydro-2H-pyran-2-yl)oxy]ethoxy}benzyl)-1H-pyrazol-3-yl]ethyl}carbamate

Cs₂CO₃ (0.18 g, 0.54 mmol) and 2-(2-bromoethoxy)tetrahydro-2H-pyran (0.10 g, 0.50 mmol) were added to a solution of the compound obtained in step a (0.21 g, 0.45 mmol) in DMF (10 mL) and the mixture was heated at 90° C. for 17 h. The reaction mixture was allowed to reach rt, poured into water (40 mL) and the aqueous layer was extracted with EtOAc (2×30 mL). The combined organic layers were dried over anhydrous Na₂SO₄, filtered and concentrated. The crude residue was purified by flash chromatography on SiO₂ (37% EtOAc/hexanes), to afford the title compound (yellow foam, 0.22 g, 83% yield).

HPLC-MS (Method B): Ret, 11.91 min; ESI⁺-MS m/z: 590 (M+1).

### c) 2-(4-{[3-(1-Aminoethyl)-1-(2,4-dichlorophenyl)-1H-pyrazol-5-yl]methyl}phenoxy)ethan-1-ol

The title compound was obtained following the procedure described in Example 24 step c, and using the compound obtained in step b as starting material.

HPLC-MS (Method I): Ret, 15.67 min; ESI⁺-MS m/z: 406 (M+1).

### Example 76. 3-Amino-3-[1-(2,4-dichlorophenyl)-5-(4-hydroxybenzyl)-1H-pyrazol-3-yl]propanoic acid

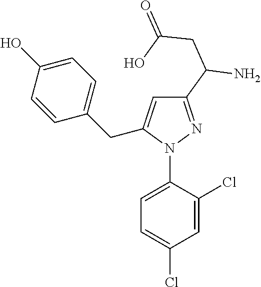 [SMILES: NC(CC(=O)O)c1cc(Cc2ccc(O)cc2)n(-c2ccc(Cl)cc2Cl)n1]

### a) 3-Amino-3-[1-(2,4-dichlorophenyl)-5-(4-methoxybenzyl)-1H-pyrazol-3-yl]propanamide

The title compound was obtained following the procedure described in Example 24 steps b-c, using N-{[1-(2,4-dichlorophenyl)-5-(4-methoxybenzyl)-1H-pyrazol-3-yl]methylene}-2-methylpropane-2-sulfinamide (Example 24, step a) and lithium dianion of N-(trimethylsilyl)acetamide as starting materials.

HPLC-MS (Method B): Ret, 9.86 min; ESI⁺-MS m/z: 419 (M+1).

### b) Title Compound

The title compound was obtained following the procedure described in Example 38 and using the compound obtained in step a as starting material.

HPLC-MS (Method I): Ret, 14.88 min; ESI⁺-MS m/z: 406 (M+1).

### Example 77. 3-Amino-3-[1-(2,4-dichlorophenyl)-5-(4-hydroxybenzyl)-1H-pyrazol-3-yl]propanamide

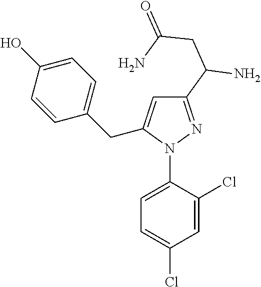 [SMILES: NC(=O)CC(N)c1cc(Cc2ccc(O)cc2)n(-c2ccc(Cl)cc2Cl)n1]

The title compound was obtained following the procedure described in Example 24 and using 1-(2,4-dichlorophenyl)-5-{4-[(tetrahydro-2H-pyran-2-yl)oxy]benzyl}-1H-pyrazole-3-carbaldehyde (prepared following the same route used for intermediates E15-E17) and lithium dianion of N-(trimethylsilyl)acetamide as starting materials.

HPLC-MS (Method I): Ret, 15.14 min; ESI⁺-MS m/z: 405 (M+1).

Table of Examples with Binding to the μ-Opioid Receptor and the α₂δ-1Subunit of the Voltage-Gated Calcium Channel:

Biological Activity

Pharmacological Study

Human α₂δ-1 Subunit of Caᵥ2.2 Calcium Channel Assay

Human α₂δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl₂ 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

Results:

As this invention is aimed at providing a compound or a chemically related series of compounds which act as dual ligands of the α₂δ subunit of voltage-gated calcium channels and the μ-opioid receptor it is a very preferred embodiment in which the compounds are selected which act as dual ligands of the α₂δ subunit of voltage-gated calcium channels and the μ-opioid receptor and especially compounds which have a binding expressed as Kᵢ responding to the following scales:

Kᵢ(μ) is preferably <1000 nM, more preferably <500 nM, even more preferably <100 nM.

Kᵢ(α₂δ-1) is preferably <10000 nM, more preferably <5000 nM, even more preferably <3000 nM or even more preferably <500 nM.

The following scale has been adopted for representing the binding to μ-opioid receptor expressed as Kᵢ:
+ K1 (μ)>=500 nM
++ 100 nM<=Kᵢ(˜)<500 nM
+++ Kᵢ(˜)<100 nM

The following scale has been adopted for representing the binding to the α₂δ-1 subunit of voltage-gated calcium channels expressed as Kᵢ:
+ Kᵢ(α₂δ-1)>=5000 nM
++ 3000 nM<=Kᵢ(α₂δ-1)<5000 nM
+++ 500 nM<=Kᵢ(α₂δ-1)<3000 nM
++++ Kᵢ(α₂δ-1)<500 nM

All compounds prepared in the present application exhibit binding to the α₂δ-1 subunit of voltage-gated calcium channels and the μ-opioid receptor, in particular the following binding results are shown:

| | α₂δ-1 | |
|---|---|---|
| EX | binding | μ binding |
| 1 | ++++ | ++ |
| 2 | +++ | + |
| 3 | ++ | + |
| 4 | +++ | ++ |
| 5 | +++ | + |
| 6 | ++ | + |
| 7 | +++ | +++ |
| 8 | +++ | + |
| 9 | +++ | + |
| 10 | +++ | + |
| 11 | +++ | + |
| 12 | +++ | + |
| 13 | +++ | + |
| 14 | +++ | + |
| 15 | +++ | + |
| 16 | +++ | + |
| 17 | ++ | ++ |
| 18 | + | + |
| 19 | +++ | + |
| 20 | ++ | + |
| 21 | ++ | + |
| 22 | ++ | + |
| 23 | +++ | + |
| 24 | +++ | ++ |
| 25 | +++ | + |
| 26 | +++ | + |
| 27 | ++++ | +++ |
| 28 | + | + |
| 29 | + | + |
| 30 | + | + |
| 31 | + | + |
| 32 | + | + |
| 33 | + | + |
| 34 | +++ | ++ |
| 35 | ++ | + |
| 36 | ++ | + |
| 37 | +++ | +++ |
| 38 | +++ | +++ |
| 39 | +++ | + |
| 40 | ++ | + |
| 41 | + | +++ |
| 42 | +++ | ++ |
| 43 | + | + |
| 44 | ++++ | + |
| 45 | +++ | + |
| 46 | ++ | + |
| 47 | +++ | + |
| 48 | +++ | + |
| 49 | + | + |
| 50 | ++ | + |
| 51 | ++++ | +++ |
| 52 | ++++ | +++ |
| 53 | ++++ | +++ |
| 54 | +++ | ++ |
| 55 | +++ | + |
| 56 | ++ | +++ |
| 57 | ++ | +++ |
| 58 | ++ | +++ |
| 59 | + | +++ |
| 60 | + | +++ |
| 61 | ++++ | +++ |
| 62 | +++ | +++ |
| 63 | + | + |
| 64 | + | + |
| 65 | ++++ | + |
| 66 | + | + |
| 67 | + | + |
| 68 | + | +++ |
| 69 | + | ++ |
| 70 | ++++ | ++ |
| 71 | + | ++ |
| 72 | + | ++ |
| 73 | + | + |
| 74 | +++ | + |
| 75 | +++ | + |
| 76 | + | ++ |
| 77 | + | +++ |

1-14. (canceled)

15. A compound of Formula (I): 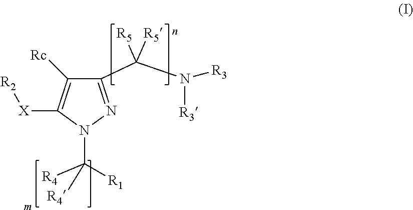
wherein
m is 0, 1, 2, 3 or 4;
n is 1, 2, 3 or 4;
X is —C(RₓR_{x′})—, —C(O)— or —O—;
R_{c} is selected from the group consisting of hydrogen, unsubstituted C₁₋₆ alkyl, unsubstituted C₂₋₆ alkenyl and unsubstituted C₂₋₆ alkynyl;
R₁ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted cycloalkyl and substituted or unsubstituted heterocyclyl;
wherein the alkyl, alkenyl or alkynyl in R₁, if substituted, is substituted with one or more substituent/s selected from the group consisting of —OR₆, —C(O)R₆, halogen, —CN, haloalkyl, haloalkoxy and —NR₆R_{6′″};
wherein the cycloalkyl aryl or heterocyclyl in R₁, if substituted, is substituted with one or more substituent/s selected from the group consisting of halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6″} are independently selected from the group consisting of hydrogen, unsubstituted C₁₋₆ alkyl, unsubstituted C₂₋₆ alkenyl and unsubstituted C₂₋₆ alkynyl;
and R_{6′″} is selected from the group consisting of hydrogen, unsubstituted C₁₋₆ alkyl, unsubstituted C₂₋₆ alkenyl, unsubstituted C₂₋₆ alkynyl and -Boc;
R₂ is selected from the group consisting of substituted or unsubstituted aryl and substituted or unsubstituted heterocyclyl;
wherein the aryl or heterocyclyl in R₂, if substituted, is substituted with one or more substituent/s selected from the group consisting of halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7′″} are independently selected from the group consisting of hydrogen, unsubstituted C₁₋₆ alkyl, unsubstituted C₂₋₆ alkenyl and unsubstituted C₂₋₆ alkynyl;
and wherein R_{7′″} is selected from the group consisting of hydrogen, unsubstituted C₁₋₆ alkyl, unsubstituted C₂₋₆ alkenyl, unsubstituted C₂₋₆ alkynyl, unsubstituted heterocyclyl, and -Boc;
R₃ and R_{3′} are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
wherein the alkyl, alkenyl or alkynyl in R₃ or R_{3′}, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R₄ and R_{4′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
R₅ and R_{5′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
Rₓ and R_{x′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
wherein, the alkyl, alkenyl or alkynyl, other than those defined in R₁, R₃ or R_{3′}, if substituted, is substituted with one or more substituent/s selected from the group consisting of —OR₉, halogen, —CN, haloalkyl, haloalkoxy, unsubstituted heterocyclyl, —C(O)OR₉, —C(O)NR₉R_{9′″} and —NR₉R_{9′″};
wherein R₉ is selected from the group consisting of hydrogen, unsubstituted C₁₋₆ alkyl, unsubstituted C₂₋₆ alkenyl, and unsubstituted C₂₋₆ alkynyl;
and wherein R_{9′″} is selected from the group consisting of hydrogen, unsubstituted C₁₋₈ alkyl, unsubstituted C₂₋₈ alkenyl, unsubstituted C₂₋₈ alkynyl and -Boc;
optionally as a stereoisomer, including enantiomers and diastereomers, a racemate or as a mixture of at least two of stereoisomers, including enantiomers and/or diastereomers, in any mixing ratio, or a corresponding salt thereof, or a corresponding solvate thereof;
with the following provisos applying:
when X is —O—, then R₁ is selected from substituted or unsubstituted aryl and substituted or unsubstituted heterocyclyl;
when X is —CH₂—, then —[C(R₄R_{4′})]ₘ—R₁ is not unsubstituted methyl;
when X is —C(O)— and m is 0, then R₂ is selected from substituted or unsubstituted monocyclic aryl and substituted or unsubstituted monocyclic aromatic heterocyclyl;
and wherein the following compound is excluded from Formula (I):

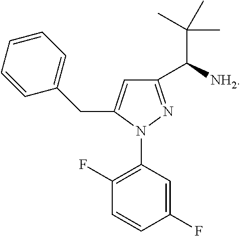 [SMILES: CC(C)(C)[C@@H](N)c1cc(Cc2ccccc2)n(-c2cc(F)ccc2F)n1]

16. A compound of Formula (I): 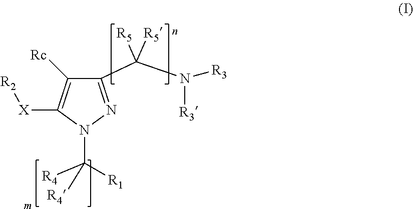
wherein
m is 0, 1, 2, 3 or 4;
n is 1, 2, 3 or 4;
X is —C(RₓR_{x′})—, —C(O)— or —O—;
R_{c} is selected from the group consisting of hydrogen, unsubstituted C₁₋₆ alkyl, unsubstituted C₂₋₆ alkenyl and unsubstituted C₂₋₆ alkynyl;
R₁ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl;
wherein the alkyl, alkenyl or alkynyl in R₁, if substituted, is substituted with one or more substituent/s selected from the group consisting of —OR₆, —C(O)R₆, halogen, —CN, haloalkyl, haloalkoxy and —NR₆R_{6′″};
wherein the aryl or heterocyclyl in R₁, if substituted, is substituted with one or more substituent/s selected from the group consisting of halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
wherein R₆, R_{6′} and R_{6′″} are independently selected from the group consisting of hydrogen, unsubstituted C₁₋₆ alkyl, unsubstituted C₂₋₆ alkenyl and unsubstituted C₂₋₆ alkynyl;
and R_{6′″} is selected from the group consisting of hydrogen, unsubstituted C₁₋₆ alkyl, unsubstituted C₂₋₆ alkenyl, unsubstituted C₂₋₆ alkynyl and -Boc;
R₂ is selected from the group consisting of substituted or unsubstituted aryl and substituted or unsubstituted heterocyclyl;
wherein the aryl or heterocyclyl in R₂, if substituted, is substituted with one or more substituent/s selected from the group consisting of halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
wherein R₇, R_{7′} and R_{7′″} are independently selected from the group consisting of hydrogen, unsubstituted C₁₋₆ alkyl, unsubstituted C₂₋₆ alkenyl and unsubstituted C₂₋₆ alkynyl;
and wherein R_{7′″} is selected from the group consisting of hydrogen, unsubstituted C₁₋₆ alkyl, unsubstituted C₂₋₆ alkenyl, unsubstituted C₂₋₆ alkynyl and -Boc;
R₃ and R_{3′} are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
wherein the alkyl, alkenyl or alkynyl in R₃ or R_{3′}, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R₄ and R_{4′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
R₅ and R_{5′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
Rₓ and R_{x′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
wherein, the alkyl, alkenyl or alkynyl, other than those defined in R₁, R₃ or R_{3′}, if substituted, is substituted with one or more substituent/s selected from the group consisting of —OR₉, halogen, —CN, haloalkyl, haloalkoxy and —NR₉R_{9′″};
wherein R₉ is selected from the group consisting of hydrogen, unsubstituted C₁₋₆ alkyl, unsubstituted C₂₋₆ alkenyl, and unsubstituted C₂₋₆ alkynyl;
and wherein R_{9′″} is selected from the group consisting of hydrogen, unsubstituted C₁₋₈ alkyl, unsubstituted C₂₋₈ alkenyl, unsubstituted C₂₋₈ alkynyl and -Boc;
optionally as a stereoisomer, including enantiomers and diastereomers, a racemate or as a mixture of at least two stereoisomers, including enantiomers and/or diastereomers, in any mixing ratio, or a corresponding salt thereof, or a corresponding solvate thereof;
with the following provisos applying:
when X is —O—, then R₁ is selected from substituted or unsubstituted aryl and substituted or unsubstituted heterocyclyl;
when X is —C(O)— and m is 0, then R₂ is selected from substituted or unsubstituted monocyclic aryl and substituted or unsubstituted monocyclic aromatic heterocyclyl;
and wherein the following compound excluded from Formula (I):

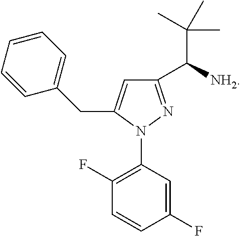 [SMILES: CC(C)(C)[C@@H](N)c1cc(Cc2ccccc2)n(-c2cc(F)ccc2F)n1]

17. The compound according to claim 15, which is a compound of formula (I′) 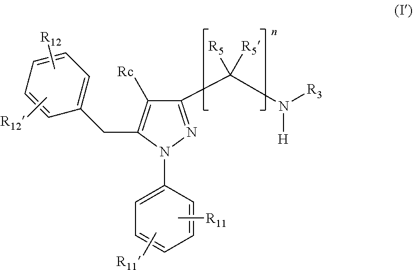
wherein
R₃ is independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
wherein the alkyl, alkenyl or alkynyl in R₃, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R₁₁ and R_{11′} are independently selected from the group consisting of hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R₁₂ and R_{12′} are independently selected from the group consisting of hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
and wherein R_{c}, R₅, R_{5′}, R₆, R_{6′}, R_{6″}, R_{6′″}, R₇, R_{7′}, R_{7″}, R_{7′″} and n are as defined in claim 15.

18. The compound according to claim 15, which is a compound of formula (I^{2′}) 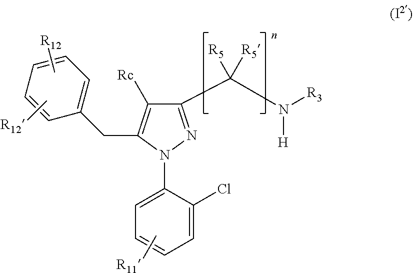
wherein
R₃ is independently selected from the group consisting of hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R_{11′} is selected from the group consisting of hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R₁₂ and R_{12′} are independently selected from the group consisting of hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
and wherein R_{c}, R₅, R_{5′}, R₆, R_{6′}, R_{6″}, R_{6′″}, R₇, R_{7′}, R_{7″}, R_{7′″} and n are as defined in claim 15.

19. The compound according to claim 15, which is a compound of formula (I^{3′}) 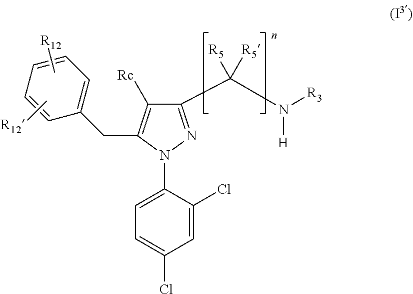
wherein
R₃ is independently selected from the group consisting of hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R₁₂ and R_{12′} are independently selected from the group consisting of hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;
and wherein R_{c}, R₅, R_{5′}, R₆, R_{6′}, R_{6″}, R_{6′″}, R₇, R_{7′}, R_{7″}, R_{7′″} and n are as defined in claim 15.

20. The compound according to claim 15, which is a compound of formula (I^{4′}) 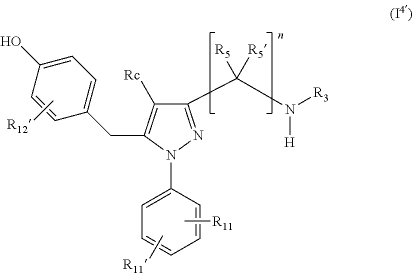
wherein
R₃ is independently selected from the group consisting of hydrogen, substituted or unsubstituted C₁₋₆ alkyl, substituted or unsubstituted C₂₋₆ alkenyl, substituted or unsubstituted C₂₋₆ alkynyl;
wherein the alkyl, alkenyl or alkynyl in R₃, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R_{11′}, is selected from the group consisting of hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R_{12′} is selected from the group consisting of hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
and wherein R_{c}, R₅, R_{5′}, R₆, R_{6′}, R_{6″}, R_{6′″}, R₇, R_{7′}, R_{7″}, R_{7′″} and n are as defined in claim 15.

21. The compound according to claim 15, which is a compound of formula (I^{5′}) 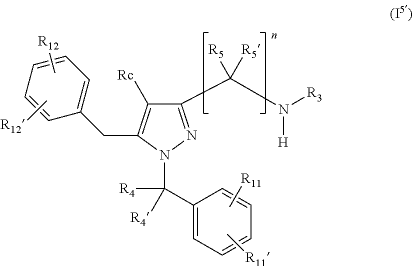
wherein
R₃ is independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
wherein the alkyl, alkenyl or alkynyl in R₃, if substituted, is substituted with one or more substituent/s selected from the group consisting of —OR₈, —C(O)R₈, halogen, —CN, haloalkyl, haloalkoxy and —NR₈R_{8′″};
wherein R₈ is selected from the group consisting of hydrogen, unsubstituted C₁₋₈ alkyl, unsubstituted C₂₋₈ alkenyl and unsubstituted C₂₋₈ alkynyl;
and wherein R_{8′″} is selected from the group consisting of hydrogen, unsubstituted C₁₋₈ alkyl, unsubstituted C₂₋₈ alkenyl, unsubstituted C₂₋₈ alkynyl and -Boc;
R₁₁ and R_{11′} are independently selected from the group consisting of hydrogen, halogen, —R₆, —OR₆, —NO₂, —NR₆R_{6′″}, NR₆C(O)R_{6′}, —NR₆S(O)₂R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and C(CH₃)₂OR₆;
R₁₂ and R_{12′} are independently selected from the group consisting of hydrogen, halogen, —R₇, —OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
and wherein R_{c}, R₄, R_{4′}, R₅, R_{5′}, R₆, R_{6′}, R_{6″}, R_{6′″}, R₇, R_{7′}, R_{7″}, R_{7′″} and n are as defined in claim 15.

22. The compound according to claim 15, which is selected from the group consisting of:
[1-(2,4-Dichlorophenyl)-5-(4-methoxybenzyl)-1H-pyrazol-3 yl]methanamine,
[1-(2-Chlorophenyl)-5-(4-methoxybenzyl)-1H-pyrazol-3-yl]methanamine,
[1-(4-Chlorophenyl)-5-(4-methoxybenzyl)-1H-pyrazol-3-yl]methanamine,
[1-(2-Chloro-4-methoxyphenyl)-5-(4-methoxybenzyl)-1H-pyrazol-3-yl]methanamine,
[1-(2-Chloro-4-fluorophenyl)-5-(4-methoxybenzyl)-1H-pyrazol-3-yl]methanamine,
4-{[3-(Aminomethyl)-1-(4-methoxyphenyl)-1H-pyrazol-5-yl]methyl}phenol,
4-{[3-(Aminomethyl)-1-(2-chloro-4-ethoxyphenyl)-1H-pyrazol-5-yl]methyl}phenol,
[1-(2,4-Dichlorophenyl)-5-(3-methoxybenzyl)-1H-pyrazol-3-yl]methanamine,
[5-Benzyl-1-(2,4-dichlorophenyl)-1H-pyrazol-3-yl]methanamine,
[5-Benzyl-1-(4-methoxyphenyl)-1H-pyrazol-3-yl]methanamine,
[1-Isobutyl-5-(4-methoxybenzyl)-1H-pyrazol-3-yl]methanamine,
[1-(4-Chlorobenzyl)-5-(4-methoxybenzyl)-1H-pyrazol-3-yl]methanamine,
[1-Benzyl-5-(4-methoxybenzyl)-1H-pyrazol-3-yl]methanamine,
[1-(2-Chlorobenzyl)-5-(4-methoxybenzyl)-1H-pyrazol-3-yl]methanamine,
[1-(2,4-Dichlorobenzyl)-5-(4-methoxybenzyl)-1H-pyrazol-3-yl]methanamine,
[5-(4-Methoxybenzyl)-1-(1-phenylethyl)-1H-pyrazol-3-yl]methanamine,
[1-(2,4-Dichlorophenyl)-5-(4-methoxyphenoxy)-1H-pyrazol-3-yl]methanamine,
N-(4-{[3-(Aminomethyl)-1-(2,4-dichlorophenyl)-1H-pyrazol-5-yl]oxy}phenyl)methanesulfonamide,
[5-(4-Methoxybenzyl)-1-phenyl-1H-pyrazol-3-yl]methanamine,
[5-(4-Methoxybenzyl)-1-(pyridin-2-yl)-1H-pyrazol-3-yl]methanamine,
1-[1-(2,4-Dichlorophenyl)-5-(4-methoxybenzyl)-1H-pyrazol-3-yl]-N,N-dimethylmethanamine,
1-[1-(2,4-Dichlorophenyl)-5-(4-methoxybenzyl)-1H-pyrazol-3-yl]-N-methylmethanamine,
N-{[1-Isobutyl-5-(4-methoxybenzyl)-1H-pyrazol-3-yl]methyl}ethanamine,
1-[1-(2,4-Dichlorophenyl)-5-(4-methoxybenzyl)-1H-pyrazol-3-yl]ethanamine,
1-[1-(2,4-Dichlorophenyl)-5-(4-ethoxybenzyl)-1H-pyrazol-3-yl]ethanamine,
N-(4-{[3-(1-Aminoethyl)-1-(2,4-dichlorophenyl)-1H-pyrazol-5-yl]methyl}phenyl)methanesulfonamide,
4-{[3-(1-Aminoethyl)-1-(2-chloro-4-methoxyphenyl)-1H-pyrazol-5-yl]methyl}phenol,
4-{[3-(1-Aminoethyl)-1-(2-chloro-5-methoxyphenyl)-1H-pyrazol-5-yl]methyl}phenol,
4-{[3-(1-Aminoethyl)-1-(4-chloro-2-methoxyphenyl)-1H-pyrazol-5-yl]methyl}phenol,
1-[1-(2,4-Dichlorophenyl)-5-(3-fluoro-4-methoxybenzyl)-1H-pyrazol-3-yl)]ethanamine,
1-[1-(2,4-Dichlorophenyl)-5-(3-fluoro-4-methoxybenzyl)-1H-pyrazol-3-yl]butan-1-amine,
1-[1-(2,4-Dichlorophenyl)-5-(4-methoxybenzyl)-1H-pyrazol-3-yl]propan-1-amine,
1-[1-(2,4-Dichlorophenyl)-5-(2-(4-methoxyphenyl)propan-2-yl)-1H-pyrazol-3-yl]ethanamine,
3-{[3-(Aminomethyl)-1-(2,4-dichlorophenyl)-1H-pyrazol-5-yl]methyl}phenol,
4-[3-(Aminomethyl)-5-benzyl-1H-pyrazol-1-yl]phenol,
3-[3-(Aminomethyl)-5-benzyl-1H-pyrazol-1-yl]phenol,
4-{[3-(Aminomethyl)-1-(2-chloro-4-methoxyphenyl)-1H-pyrazol-5-yl]methyl}phenol,
4-{[3-(Aminomethyl)-1-(2,4-dichlorophenyl)-1H-pyrazol-5-yl]methyl}phenol,
4-{[3-(Aminomethyl)-1-(2-chlorophenyl)-1H-pyrazol-5-yl]methyl}phenol,
4-{[3-(Aminomethyl)-1-(4-chlorophenyl)-1H-pyrazol-5-yl]methyl}phenol,
4-[3-(Aminomethyl)-5-(4-hydroxybenzyl)-1H-pyrazol-1-yl]-3-chlorophenol,
4-{[3-(Aminomethyl)-1-(2-chloro-4-fluorophenyl)-1H-pyrazol-5-yl]methyl}phenol,
4-{[3-(Aminomethyl)-1-(pyridin-2-yl)-1H-pyrazol-5-yl]methyl}phenol,
4-{[3-(Aminomethyl)-1-isobutyl-1H-pyrazol-5-yl]methyl}phenol,
4-{[3-(Aminomethyl)-1-(4-chlorobenzyl)-1H-pyrazol-5-yl]methyl}phenol,
4-{[3-(Aminomethyl)-1-benzyl-1H-pyrazol-5-yl]methyl}phenol,
4-{[3-(Aminomethyl)-1-(2-chlorobenzyl)-1H-pyrazol-5-yl]methyl}phenol,
4-{[3-(Aminomethyl)-1-(2-chlorobenzyl)-1H-pyrazol-5-yl]methyl}phenol,
4-{[3-(Aminomethyl)-1-(1-phenylethyl)-1H-pyrazol-5-yl]methyl}phenol,
4-({3-[(Ethylamino)methyl]-1-isobutyl-1H-pyrazol-5-yl}methyl)phenol,
4-{[3-(1-Aminoethyl)-1-(2,4-dichlorophenyl)-1H-pyrazol-5-yl]methyl}phenol,
4-{[3-(1-Aminoethyl)-1-(2,4-dichlorophenyl)-1H-pyrazol-5-yl]methyl}-2-fluorophenol,
4-{[3-(1-Aminobutyl)-1-(2,4-dichlorophenyl)-1H-pyrazol-5-yl]methyl}-2-fluorophenol,
4-{[3-(1-Aminopropyl)-1-(2,4-dichlorophenyl)-1H-pyrazol-5-yl]methyl}phenol,
4-{2-[3-(1-Aminoethyl)-1-(2,4-dichlorophenyl)-1H-pyrazol-5-yl]propan-2-yl}phenol,
4-{[3-(2-Aminopropan-2-yl)-1-(2,4-dichlorophenyl)-1H-pyrazol-5-yl]methyl}-2-fluorophenol,
4-{[3-(2-Aminopropan-2-yl)-1-(2,4-dichlorophenyl)-1H-pyrazol-5-yl]methyl}phenol,
4-{[3-(2-Aminoethyl)-1-(2,4-dichlorophenyl)-1H-pyrazol-5-yl]methyl}phenol,
4-{[3-(1-Aminopropan-2-yl)-1-(2,4-dichlorophenyl)-1H-pyrazol-5-yl]methyl}phenol,
4-{[3-(1-Aminobutan-2-yl)-1-(2,4-dichlorophenyl)-1H-pyrazol-5-yl]methyl}phenol,
(S)-4-{[3-(1-Aminoethyl)-1-(2,4-dichlorophenyl)-1H-pyrazol-5-yl]methyl}phenol,
(R)-4-{[3-(1-Aminoethyl)-1-(2,4-dichlorophenyl)-1H-pyrazol-5-yl]methyl}phenol,
(S)-4-{[3-(1-Aminoethyl)-1-(2,4-dichlorophenyl)-1H-pyrazol-5-yl]methyl}-2-fluorophenol, and
(R)-4-{[3-(1-Aminoethyl)-1-(2,4-dichlorophenyl)-1H-pyrazol-5-yl]methyl}-2-fluorophenol.

23. The compound according to claim 15, which is selected from the group consisting of:
4-{[3-(1-Aminoethyl)-1-cyclohexyl-1H-pyrazol-5-yl]methyl}phenol,
4-{[3-(1-Aminoethyl)-1-(tetrahydro-2H-pyran-4-yl)-1H-pyrazol-5-yl]methyl}phenol,
4-{[3-(1-Aminoethyl)-1-phenethyl-1H-pyrazol-5-yl]methyl}phenol,
4-{[3-(2-Aminopropyl)-1-(2,4-dichlorophenyl)-1H-pyrazol-5-yl]methyl}phenol,
(R)-4-{[3-(1-Aminoethyl)-1-(2-chloro-4-methoxyphenyl)-1H-pyrazol-5-yl]methyl}phenol,
(S)-4-{[3-(1-Aminoethyl)-1-(2-chloro-4-methoxyphenyl)-1H-pyrazol-5-yl]methyl}phenol,
4-{[3-(1-Amino-2-hydroxyethyl)-1-(2,4-dichlorophenyl)-1H-pyrazol-5-yl]methyl}phenol,
4-({3-[1-Amino-2-(piperidin-1-yl)ethyl]-1-(2,4-dichlorophenyl)-1H-pyrazol-5-yl}methyl)phenol,
4-{[3-(1-Amino-2-morpholinoethyl)-1-(2,4-dichlorophenyl)-1H-pyrazol-5-yl]methyl}phenol,
N-(4-{[3-(Aminomethyl)-1-(2,4-dichlorophenyl)-1H-pyrazol-5-yl]methyl}phenyl)thiazol-2-amine,
2-(4-{[3-(1-Aminoethyl)-1-(2,4-dichlorophenyl)-1H-pyrazol-5-yl]methyl}phenoxy)ethan-1-ol,
3-Amino-3-[1-(2,4-dichlorophenyl)-5-(4-hydroxybenzyl)-1H-pyrazol-3-yl]propanoic acid, and
3-Amino-3-[1-(2,4-dichlorophenyl)-5-(4-hydroxybenzyl)-1H-pyrazol-3-yl]propanamide.

24. A process for the preparation of a compound of Formula (I) according to claim 15, wherein X is —C(RₓR_{x′})— or —O—, R₅ and R_{5′} are both hydrogen and n is 1, 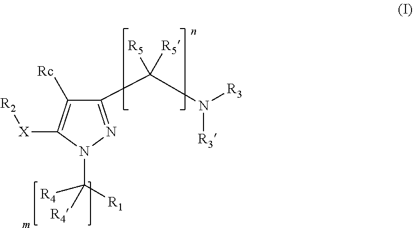
which process comprises reductive amination of a compound of formula XIVex, 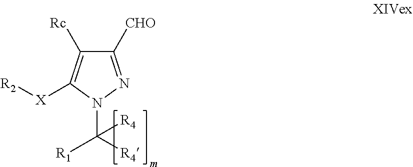
with an amine of formula XV
HNR₃R₃′ XV;
OR
for the production of a compound of Formula (I), wherein X is —C(RₓR_{x′})— or —O—, R₅, is hydrogen and n is 1, 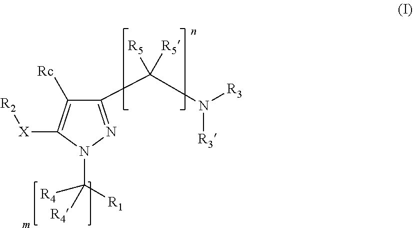
which process comprises addition of an organometallic reagent of formula XVIII
R₅MgBr XVIII
to a compound of formula XVIIex 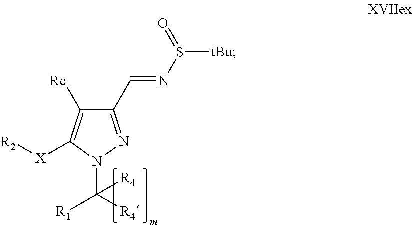
OR
for the production of a compound of Formula (I), wherein X is —C(RₓR_{x′})— or —O— and n is 1, 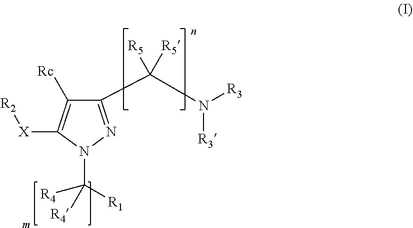
which process comprises an alkylation reaction of a compound of formula XXex 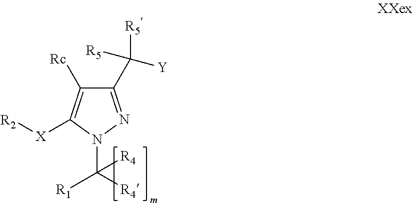
with an amine of formula XV
HNR₃R₃′ XV;
OR
for the production of a compound according to Formula (I), wherein the compound of Formula (I) is a compound of Formula (Idₑₓ) and wherein X is —C(RₓR_{x′})— or —O—, 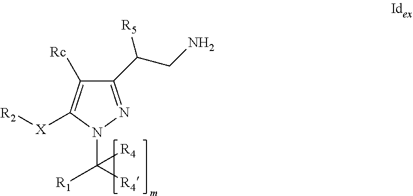
which process comprises reaction of a compound of formula XXIex, 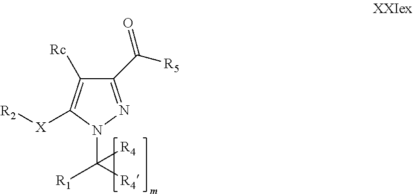
with tosylmethylisocyanide;
OR
for the production of a compound according to Formula (I), wherein the compound of Formula (I) is a compound of Formula (Ieₑₓ) and wherein X is —C(RₓR_{x′})— or —O—, 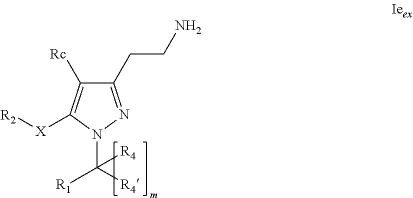
which process comprises the reduction of compounds of formula XXIIex 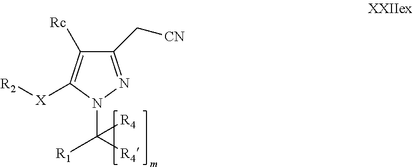
with a suitable reducing agent,
wherein, unless otherwise defined, R₁, R₂, R_{c}, R₃, R_{3′}, R₄, R_{4′}, R₅, R_{5′}Rₓ, R_{x′}, m, and n are as defined in claim 15.

25. A process for the preparation of a compound of Formula (I) according to claim 15, employing a compound of Formula Iₑₓ, IIa, IIb, III, IV, V, VI, VII, VIII, IX, X, XI, XII, XIII, XIV, XIVₑₓ, XV, XVI, XVIₑₓ, XVII, XVIIₑₓ, XVIII, XIX, XX, XXₑₓ, XXI, XXIₑₓ or XXII, XXIIₑₓ, 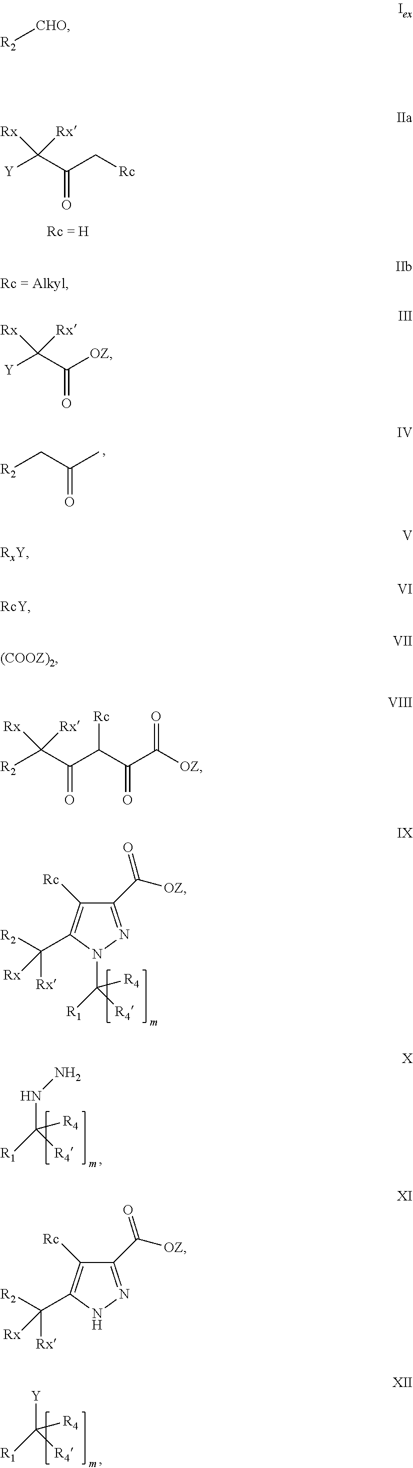 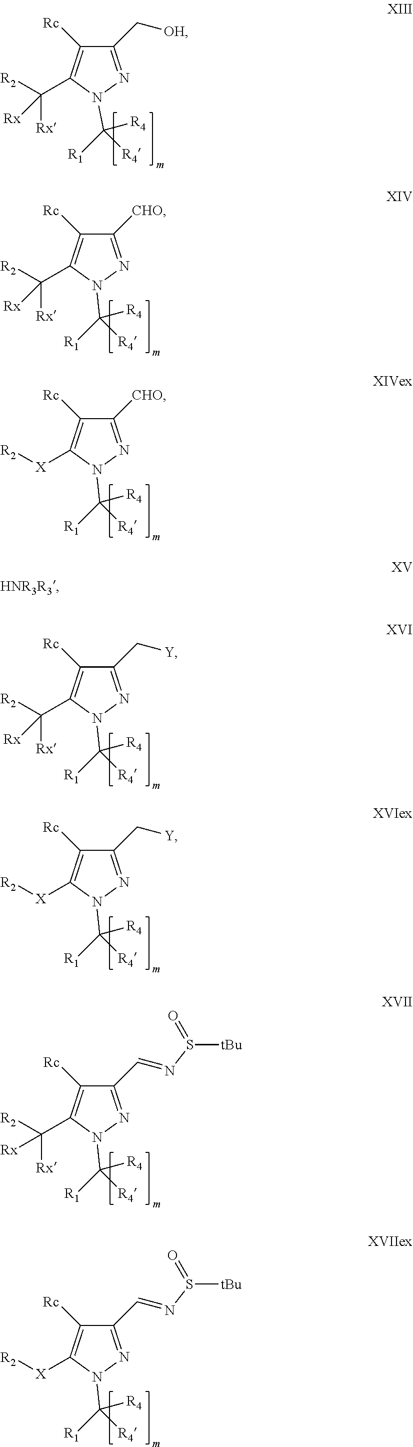 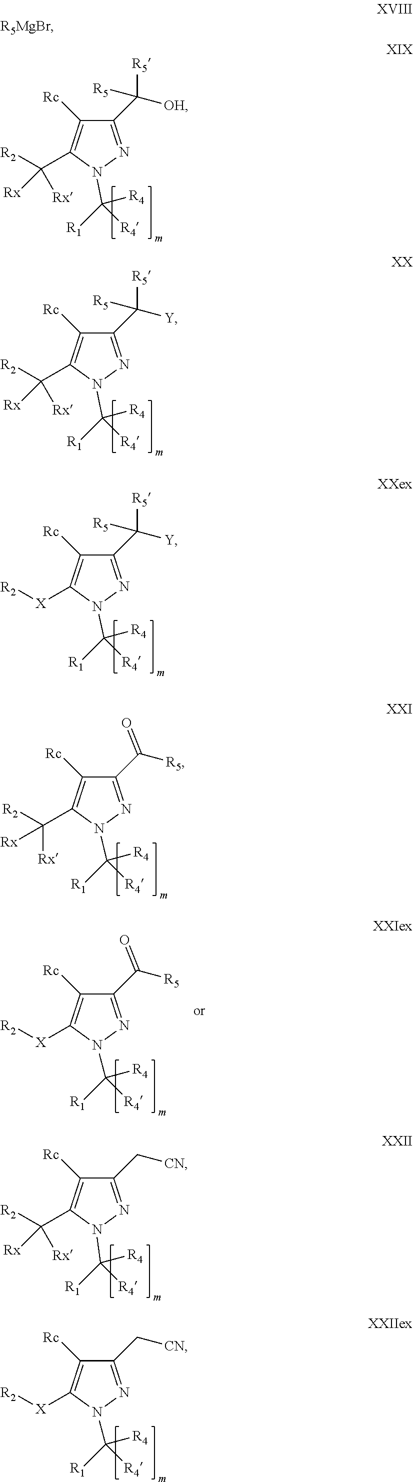
wherein R₁, R₂, R_{c}, R₃, R_{3′}, R₄, R_{4′}, R₅, R_{5′}, Rₓ, R_{x′}, X, m, and n are as defined in claim 15, Z is an alkyl group, and Y is a leaving group, including halogen.

26. A pharmaceutical composition which comprises the compound according to claim 15, or a pharmaceutically acceptable salt thereof, and a pharmaceutically acceptable carrier, adjuvant or vehicle.

27. A method of treating pain in a subject in need thereof, comprising administration of an effective amount of the compound according to claim 15.

28. The method according to claim 27, wherein the pain is selected from the group consisting of medium to severe pain, visceral pain, chronic pain, cancer pain, migraine, inflammatory pain, acute pain or neuropathic pain, allodynia, and hyperalgesia.

29. A pharmaceutical composition which comprises the compound according to claim 16, or a pharmaceutically acceptable salt thereof, and a pharmaceutically acceptable carrier, adjuvant or vehicle.

30. A method of treating pain in a subject in need thereof, comprising administration of an effective amount of the compound according to claim 16.

31. The method according to claim 30, wherein the pain is selected from the group consisting of medium to severe pain, visceral pain, chronic pain, cancer pain, migraine, inflammatory pain, acute pain or neuropathic pain, allodynia, and hyperalgesia.
